(19) United States
(12) Patent Application Publication
(10) Pub. No.: US 2026/0125647 A1
(43) Pub. Date: May 7, 2026

(54) AGE-MODULATING COMPOUNDS AND METHODS FOR MAKING AGE-MODULATED CELLS

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Nathalie SAURAT, Long Island City, NY (US); Lorenz Studer, New York, NY (US)

(73) Assignees: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(21) Appl. No.: 19/438,959

(22) Filed: Jan. 2, 2026

Related U.S. Application Data
(63) Continuation of application No. PCT/US2024/036791, filed on Jul. 3, 2024.
(60) Provisional application No. 63/524,801, filed on Jul. 3, 2023.

(51) Int. Cl. C12N 5/077; C12N 5/0793

(52) U.S. Cl. CPC C12N 5/0656; C12N 5/0619; C12N 2501/065; C12N 2501/405; C12N 2501/81; C12N 2503/02

(57) ABSTRACT

Provided are age-modulated cells and method for making age-modulated cells. The aging and rejuvenation processes can be induced for young, aged, mature and/or immature cells, such as a somatic cell, a stem cell, a stem cell-derived somatic cell, including an induced pluripotent stem cell-derived cell, by contacting cells with one or more age-inducing or rejuvenating agent. Methods described by the present disclosure can produce age-appropriate cells from a somatic cell or a stem cell, such as an old cell, young cell, immature cell, and/or a mature cell. Such age-modified cells constitute model systems for the study of late-onset diseases and/or disorders.

### CROSS REFERENCE TO RELATED APPLICATIONS

This application is a continuation of International Patent Application No. PCT/US2024/036791, filed Jul. 3, 2024, which claims priority to U.S. Provisional Application No. 63/524,801 filed Jul. 3, 2023, which are hereby incorporated by reference in their entirety, and to which priority is claimed.

### 1. TECHNICAL FIELD

The present disclosure relates to methods for modulating cellular aging and/or progression of neurodegenerative diseases (e.g., Alzheimers Disease), and age-modulated cells which can be used both clinically as well as in basic research. Furthermore, the present disclosure also relates to methods of reversing cellular aging (i.e., cellular rejuvenation).

### 2. BACKGROUND OF THE DISCLOSURE

hPSC technology offers access to a diverse range of cell types for disease modelling. However, hPSC-derived lineages represent fetal rather than adult or aged states. This poses significant challenges when modelling age-dependent disorders because aging increases the risk of disease across tissue types. This challenge is especially salient to neurons where access to primary tissue is limited, and replicative senescence can't be used to model cellular aging. Together, these factors have led to a major interest in developing strategies to fast-forward cellular age in hPSC-derived cells. In neurons, these efforts can be grouped into two broad approaches: induced aging of developmentally specified hPSC-derived neurons versus transdifferentiation of aged fibroblasts directly to neurons. Induced aging approaches take advantage of the ability to scale, to direct cell type specificity and to apply the range of cellular engineering technologies inherent to the hPSC disease modelling field, but there is no consensus on the most relevant strategies to trigger aging-like states in hPSC-derive lineages. The transdifferentiation of fibroblasts directly to neurons maintains any age-related signature present in the primary fibroblast but this strategy involves several technical challenges including low transdifferentiation efficiency, scalability, and stability of the resulting neurons. Furthermore, recent studies indicate that aging may show tissue and cell type specificity raising the question of how relevant fibroblast aging signatures are to neuronal age.

To compare, evaluate and expand on any age-modifying interventions it is essential to develop methods that quantitatively measure biological age at the cellular level. A commonly used approach are DNA methylation-based aging clocks that are based on 5mC methylation status at tissue- and age-related CpG sites. In addition, there are transcriptomics-based aging clocks which offer an obvious advantage due to the broad availability of data sets spanning many cell types, disease conditions and cellular states. Transcriptomic clocks may also provide more direct mechanistic insights into the aging process than epigenetic measurements. There have been several efforts to develop both global and cell type specific transcriptional aging clocks. To date, the use of aging clocks has been limited to estimating absolute or relative cellular age. An exciting future application is their use as a direct readout in functional screens aimed at age manipulation to establish improved hPSC-based models of late onset human disease.

There is need to identify novel regulators of age that could be incorporated into hPSC-based models of neurodegenerative disease to accelerate the acquisition of late onset phenotypes in vitro, and to develop novel methods to score changes in the relative age of human cells. Furthermore, there is need to develop cellular models that more faithfully recapitulate late-onset disease phenotypes in vitro.

### 3. SUMMARY OF THE DISCLOSURE

The present disclosure provides method for inducing cellular aging in a cell or population of cells, comprising contacting the cell or population of cells with one or more age-inducing agent. In certain embodiments, the cell or population of cells comprise fibroblasts. In certain embodiments, the one or more age-inducing agent is selected from the group consisting of one or more cyclin-dependent kinase (CDK) inhibitor, one or more adenosine kinase inhibitor, one or more topoisomerase II inhibitor, one or more protein kinase C (PKC) inhibitor, one or more DNA binding agent, and combinations thereof. In certain embodiments, the one or more CDK inhibitor comprises alvocidib. In certain embodiments, the one or more adenosine kinase inhibitor comprises 5-iodotubercin. In certain embodiments, the one or more topoisomerase II inhibitor comprises mitoxantrone. In certain embodiments, the one or more PKC inhibitor comprises bisindolylmaleimide-ix. In certain embodiments, the one or more DNA binding agent comprises chromomycin-a3. In certain embodiments, the cell or population of cells comprise neurons, motoneurons, cortical neurons, peripheral sensory neurons, or midbrain dopamine neurons. In certain embodiments, the one or more age-inducing agent is selected from the group consisting of one or more DNA synthesis inhibitor, one or more microtubule antagonist, one or more Sirtuin-2 (SIRT2) inhibitor, BRD-K25731886, doxercalciferol, and combinations thereof. In certain embodiments, the one or more DNA synthesis inhibitor comprises fludarabine. In certain embodiments, the one or more microtubule antagonist comprises vinorelbine. In certain embodiments, the one or more SIRT2 inhibitor comprises AGK2. In certain embodiments, contacting the cell or population of cells with one or more age-inducing agent increases the expression level or presence in the cell or population of cells of one or more chronological marker signature of an old cell. In certain embodiments, contacting the cell or population of cells with one or more age-inducing agent decreases the expression level or presence in the cell or population of cells of one or more chronological marker signature of a young cell. In certain embodiments, contacting the cell or population of cells with one or more age-inducing agent induces loss of heterochromatin, disrupted nuclear lamina, increased DNA damage, increased cellular senescence, disrupted proteostasis and increased dependence on autophagy, increased caveolin-1 (CAV1) expression, or a combination thereof. In certain embodiments, the cell or population of cells are derived from primary cells, fetal cells, pluripotent stem cells, embryonic stem cells, induced pluripotent stem cells, or combinations thereof. In certain embodiments, the cell or population of cells are human.

The present disclosure further provides a method for rejuvenating cellular age in a cell or population of cells, comprising contacting the cell or population of cells with one or more rejuvenating agent. In certain embodiments, the cell or population of cells comprise fibroblasts. In certain embodiments, the one or more rejuvenating agent is selected from the group consisting of one or more histone deacetylase (HDAC) inhibitor, one or more heat shock protein (HSP) inhibitor, one or more adrenergic receptor antagonist, one or more phosphoinositide 3-kinase (PI3K) inhibitor, and combinations thereof. In certain embodiments, the one or more HDAC inhibitor comprises mocetinostat. In certain embodiments, the one or more HSP inhibitor comprises radicicol, geldanamycin, or a combination thereof. In certain embodiments, the one or more adrenergic receptor antagonist comprises alfuzosin. In certain embodiments, the one or more PI3K inhibitor comprises wortmannin. In certain embodiments, the cell or population of cells comprise neurons, motoneurons, cortical neurons, peripheral sensory neurons, or midbrain dopamine neurons. In certain embodiments, the one or more rejuvenating agent is selected from the group consisting of one or more Sirtuin (SIRT) activator, one or more HDAC inhibitor, one or more poly ADP ribose polymerase (PARP) inhibitor, one or more ataxia telangiectasia and Rad3-related (ATR) signaling inhibitor, and combinations thereof. In certain embodiments, the one or more SIRT activator comprises resveratrol. In certain embodiments, the one or more HDAC inhibitor comprises SB-939, BRD-A19037878, or a combination thereof. In certain embodiments, the PARP inhibitor comprises veliparib. In certain embodiments, the ATR signaling inhibitor comprises VE-821. In certain embodiments, contacting the cell or population of cells with one or more rejuvenating agent increases the expression level or presence in the cell or population of cells of one or more chronological marker signature of a young cell. In certain embodiments, contacting the cell or population of cells with one or more rejuvenating agent decreases the expression level or presence in the cell or population of cells of one or more chronological marker signature of an old cell. In certain embodiments, contacting the cell or population of cells with one or more rejuvenating agent reduces loss of heterochromatin, disrupted nuclear lamina, DNA damage, cellular senescence, disrupted proteostasis and dependence on autophagy, caveolin-1 (CAV1) expression, or a combination thereof. In certain embodiments, the cell or population of cells are derived from primary cells, fetal cells, pluripotent stem cells, embryonic stem cells, induced pluripotent stem cells, or combinations thereof. In certain embodiments, the cell or population of cells are human.

The present disclosure further provides an age-induced cell, wherein the age-induced cell is produced by a method comprising contacting a cell with one or more age-inducing agent. In certain embodiments, the age-induced cell is derived from fibroblasts. In certain embodiments, the one or more age-inducing agent is selected from the group consisting of one or more cyclin-dependent kinase (CDK) inhibitor, one or more adenosine kinase inhibitor, one or more topoisomerase II inhibitor, one or more protein kinase C (PKC) inhibitor, one or more DNA binding agent, and combinations thereof. In certain embodiments, the one or more CDK inhibitor comprises alvocidib. In certain embodiments, the one or more adenosine kinase inhibitor comprises 5-iodotubercin. In certain embodiments, the one or more topoisomerase II inhibitor comprises mitoxantrone. In certain embodiments, the one or more PKC inhibitor comprises bisindolylmaleimide-ix. In certain embodiments, the one or more DNA binding agent comprises chromomycin-a3. In certain embodiments, the age-induced cell is derived from neurons, motoneurons, cortical neurons, peripheral sensory neurons, or midbrain dopamine neurons. In certain embodiments, the one or more age-inducing agent is selected from the group consisting of one or more DNA synthesis inhibitor, one or more microtubule antagonist, one or more Sirtuin-2 (SIRT2) inhibitor, BRD-K25731886, doxercalciferol, and combinations thereof. In certain embodiments, the one or more DNA synthesis inhibitor comprises fludarabine. In certain embodiments, the one or more microtubule antagonist comprises vinorelbine. In certain embodiments, the one or more SIRT2 inhibitor comprises AGK2. In certain embodiments, the age-induced cell is derived from primary cells, fetal cells, pluripotent stem cells, embryonic stem cells, induced pluripotent stem cells, or combinations thereof. In certain embodiments, the age-induced cell is human. In certain embodiments, the cell with one or more age-inducing agent increases the expression level or presence of one or more chronological marker signature of an old cell. In certain embodiments, contacting the cell with one or more age-inducing agent decreases the expression level or presence of one or more chronological marker signature of a young cell. In certain embodiments, contacting the cell with one or more age-inducing agent induces loss of heterochromatin, disrupted nuclear lamina, increased DNA damage, increased cellular senescence, disrupted proteostasis and increased dependence on autophagy, increased caveolin-1 (CAV1) expression, or a combination thereof.

The present disclosure further provides a rejuvenated cell, wherein the rejuvenated cell is produced by a method comprising contacting a cell with one or more rejuvenating agent. In certain embodiments, the rejuvenated cell is derived from fibroblasts. In certain embodiments, the one or more rejuvenating agent is selected from the group consisting of one or more histone deacetylase (HDAC) inhibitor, one or more heat shock protein (HSP) inhibitor, one or more adrenergic receptor antagonist, one or more phosphoinositide 3-kinase (PI3K) inhibitor, and combinations thereof. In certain embodiments, the one or more HDAC inhibitor comprises mocetinostat. In certain embodiments, the one or more HSP inhibitor comprises radicicol, geldanamycin, or a combination thereof. In certain embodiments, the one or more adrenergic receptor antagonist comprises alfuzosin. In certain embodiments, the one or more PI3K inhibitor comprises wortmannin. In certain embodiments, the rejuvenated cell is derived from neurons, motoneurons, cortical neurons, peripheral sensory neurons, or midbrain dopamine neurons. In certain embodiments, the one or more rejuvenating agent is selected from the group consisting of one or more Sirtuin (SIRT) activator, one or more HDAC inhibitor, one or more poly ADP ribose polymerase (PARP) inhibitor, one or more ataxia telangiectasia and Rad3-related (ATR) signaling inhibitor, and combinations thereof. In certain embodiments, the one or more SIRT activator comprises resveratrol. In certain embodiments, the one or more HDAC inhibitor comprises SB-939, BRD-A19037878, or a combination thereof. In certain embodiments, the PARP inhibitor comprises veliparib. In certain embodiments, the ATR signaling inhibitor comprises VE-821. In certain embodiments, the rejuvenated cell is derived from primary cells, fetal cells, pluripotent stem cells, embryonic stem cells, induced pluripotent stem cells, or combinations thereof. In certain embodiments, the rejuvenated cell is human. In certain embodiments, contacting the cell with one or more rejuvenating agent increases the expression level or presence of one or more chronological marker signature of a young cell. In certain embodiments, contacting the cell with one or more rejuvenating agent decreases the expression level or presence of one or more chronological marker signature of an old cell. In certain embodiments, contacting the cell with one or more rejuvenating agent reduces loss of heterochromatin, disrupted nuclear lamina, DNA damage, cellular senescence, disrupted proteostasis and dependence on autophagy, caveolin-1 (CAV1) expression, or a combination thereof.

The present disclosure further provides a composition comprising the age-induced cell disclosed herein or the rejuvenated cell disclosed herein. In certain embodiments, the composition is a pharmaceutical composition. In certain embodiments, the composition further comprises a pharmaceutically acceptable carrier.

The present disclosure provides a method of modeling neurodegenerative disease, comprising: contacting a cell or population of cells with one or more age-inducing agent to obtain an age-modulated cell or population of age-modulated cells; and contacting the age-modulated cell or population of age-modulated cells with one or more candidate compound. In certain embodiments, the method further comprises detecting an alteration in at least one of the survival, biological activity, structure or morphology of the age-modulated cell or population of age-modulated cells. In certain embodiments, the method further comprises selecting as the drug a candidate one or more compound that alters at least one of the survival, biological activity, structure or morphology of the age-modulated cell or population of age-modulated cells. In certain embodiments, the neurodegenerative disease is Parkinson's disease (PD) or Alzheimer's Disease (AD).

The present disclosure further provides a kit for inducing cellular aging in a cell or population of cells, comprising one or more age-inducing agent is selected from the group consisting of one or more DNA synthesis inhibitor, one or more microtubule antagonist, one or more SIRT2 inhibitor, BRD-K25731886, doxercalciferol, one or more CDK inhibitor, one or more adenosine kinase inhibitor, one or more topoisomerase II inhibitor, one or more PKC inhibitor, one or more DNA binding agent, and combinations thereof. In certain embodiments, the one or more DNA synthesis inhibitor comprises fludarabine. In certain embodiments, the one or more microtubule antagonist comprises vinorelbine. In certain embodiments, the one or more SIRT2 inhibitor comprises AGK2. In certain embodiments, the one or more CDK inhibitor comprises alvocidib. In certain embodiments, the one or more adenosine kinase inhibitor comprises 5-iodotubercin. In certain embodiments, the one or more topoisomerase II inhibitor comprises mitoxantrone. In certain embodiments, the one or more PKC inhibitor comprises bisindolylmaleimide-ix. In certain embodiments, the one or more DNA binding agent comprises chromomycin-a3. In certain embodiments, the kit further comprises instructions for contacting a cell or population of cells with the one or more age-inducing agent to induce expression in the cell or population of cells of one or more chronological marker signature of an old cell.

The present disclosure further provides a kit for rejuvenating a cell or population of cells, comprising one or more rejuvenating agent selected from the group consisting of one or more histone deacetylase (HDAC) inhibitor, one or more heat shock protein (HSP) inhibitor, one or more adrenergic receptor antagonist, one or more phosphoinositide 3-kinase (PI3K) inhibitor, one or more Sirtuin (SIRT) activator, one or more HDAC inhibitor, one or more poly ADP ribose polymerase (PARP) inhibitor, one or more ataxia telangiectasia and Rad3-related (ATR) signaling inhibitor, and combinations thereof. In certain embodiments, the one or more HDAC inhibitor comprises mocetinostat. In certain embodiments, the one or more HSP inhibitor comprises radicicol, geldanamycin, or a combination thereof. In certain embodiments, the one or more adrenergic receptor antagonist comprises alfuzosin. In certain embodiments, the one or more PI3K inhibitor comprises wortmannin. In certain embodiments, the one or more SIRT activator comprises resveratrol. In certain embodiments, the one or more HDAC inhibitor comprises SB-939, BRD-A19037878, or a combination thereof. In certain embodiments, the PARP inhibitor comprises veliparib. In certain embodiments, the ATR signaling inhibitor comprises VE-821. In certain embodiments, the kit further comprises instructions for contacting a cell or population of cells with the one or more rejuvenating agent to induce expression in the cell or population of cells of one or more chronological marker signature of a young cell.

### 4. BRIEF DESCRIPTION OF THE DRAWINGS

FIGS. 1A-1F show identification of transcriptional signatures of age in primary fibroblasts, frontal cortex and substantia nigra. A) Schematic outlining of transcriptional profiles of primary cell lines (fibroblasts; pFIB) and tissues (frontal cortex; F. Cortex and substantia nigra; S. nigra) from young and old donors sequenced in this study. B) PCA of RNA-seq data from primary fibroblasts, frontal cortex and substantia nigra from young and old donors. C) Volcano plots showing significantly differentially expressed genes (FDR<0.1) between young and old primary tissues. D) Functionally enriched pathways of age-associated genes in each tissue (selected terms) and their respective Normalized Enrichment Score (NES). E) Heatmap indicating directionality of the change in gene expression in young (“Y”) vs old (“O”) primary tissue samples. Darker shades indicate that change is significant (FDR<0.05). RNA-seq datasets generated as part of this study were: Total SN (substantia nigra), Total FC (frontal cortex), Total pF (total RNA of primary fibroblast) and PolyA pF (poly-A selection of primary fibroblast). External datasets were PolyA Gage FC (poly-A selection of frontal cortex) and PolyA Gage pF (polyA selection of primary fibroblast). F) Top 50 genes for each subcategory of aging genes ranked according to combined p-value.

FIGS. 2A-2D show establishment and validation of RNAge score. A) Schema outlining the steps to calculate the RNAge score. Welch's two-sample t tests are calculated on the normalized values of the top 100 age signature genes (FIG. 1E) between two conditions. The T-scores are weighted multiplying by a binary marker vector that signifies the directionality of the expected expression changes (i.e. negative for genes marking older samples and positive for genes expressed highly in younger samples) and the mean values is the final age score. Percentage of common genes is the fraction of genes with positive weighted t-score. B) RNAge scores of the datasets used to establish aging signature subcategories. RNA-seq data used in Total pF, PolyA pF, Total FC and Total SN were generated as part of this study. Gage pF and Gage FC are similar young and old samples that were profiled independently in a different study 13. C) Independent validation of the RNAge score for pF, FC, Cortex, SN, and mouse brain from additional external studies. Tissue type, species and data source are indicated on the bubble plot. D) Application of RNAge score to young and old human primary stomach and human primary cerebellum. Data was externally generated. RNAge score refers to the modified t-score for each pairwise comparison. The size of the bubbles indicates the percentage of genes that make up the aging score that change in the expected direction for young vs old.

FIGS. 3A-3E show that RNAge score can be used to measure cellular rejuvenation. A) Experimental schema of Fibroblast rejuvenation experiment. Primary fibroblasts (pFIB; pF) from young and old donors were first reprogrammed to pluripotent stem cells (iPSC) then re-differentiated into iPSC derived fibroblasts (iPSC-FIB). RNA, 5mC and 5hmC was performed at each step. B) Morphology, immunofluorescence for the fibroblast markers COL1A and VIM and flow cytometry for CD-13 and HLA-ABC in primary fibroblasts, iPSCs generated from these fibroblasts and in matched iPSC-derived fibroblasts. C) RNA-seq analysis showing that gene expression of fibroblast specific genes in iPSC-FIB closely resembles that of pFIB indicating the re-establishment of fibroblast identity in iPSC-FIB. (N=6 independent cell lines at each stage; 3 derived from young donors and 3 derived from old donors). D) Volcano plots showing DEGs (FDR<0.1) between young and old pFibs, iPSCs derived from young and old donors and iPSC-FIB generated from these stem cells. The loss of aged signature (rejuvenation) is indicated by the reduction in the number of significantly differentiated genes in iPSC-FIB compared to pF. (N=3 independent cell lines derived from young donors and N=3 derived from old donors). E) RNAge score comparing the relative age of the iPSC-FIBs (iF) to their matched pFIBs (pF) or direct comparison of the relative age of iFs from young or old donors. The largest RNAge score are when comparing iPSC-FIB to their matched primary fibroblast, whereas RNAge score from young vs. old iPSC-FIB is minimal (N=3 independent cell lines derived from young donors and N=3 derived from old donors)

FIGS. 4A-4D show RNAge score in accelerated and induced aging models. A) PCA of RNA-seq data from primary fibroblasts originating from young, old or individuals with HGPS. B) RNAge score of young pFIBs vs. HGPS pFIBs form this study data (top) and similar comparison using RNA-seq data from Fleischer et. al., 2018 (bottom) demonstrating that HGPS do not mimic natural aging. C) RNAge score of iPSC derived induced neurons (iPSCiN) vs. induced neurons derived from primary fibroblasts (pFIB-iN) from Mertens et al., 2015 indicating that trans-differentiated neurons from aged fibroblasts maintain both neuronal and fibroblast aging. D) RNAge measurement in PSC-derived neurons in response to multiple published induced aging strategies; Progerin expression, SATB1-KO, and SLO (Miller et al 2013, Riessland et al 2019 and Fathi et al 2022).

FIGS. 5A-5E show identification of novel regulators of cellular age using RNAge score. A) Analysis workflow of the L1000 screen of the genetic and drug perturbation data that induce aging signature using RNAge scoring. B) Distribution of the RNAge scores for all drug-based perturbations in the L1000 library that were performed in fibroblasts (1HAE; left panel) and neurons (NEU; right panel). The top ranked compounds according to the pan aging and fibroblasts scores are indicated on the fibroblast (1HAE) plot and the top ranked compounds according to the pan aging and pan-neuronal scores are indicated on the neurons (NEU) plot (see points and listed compound names). Perturbations selected for experimental validation are in bold. C-E) To select the optimal concentration for age induction (or rejuvenation) drug treatment, RNAge scores were calculated for all L1000 experiments utilizing the candidate drugs by concentration and duration of treatment. RNAge scores were calculated for candidate rejuvenation drugs in fibroblasts (C), candidate aging inducers in fibroblasts (D), and candidate aging inducers in neurons (E). From the RNAge score distribution for the various aging signatures and cell types the optimal concentration were selected for experimental validation.

FIGS. 6A-6C show experimental validation top scoring modulators of RNAge. A) PCA of RNA-seq data from old fibroblasts treated with candidate rejuvenating compounds and respective controls and young fibroblasts and neurons treated with candidate age inducers. B) RNAge score of old and fetal fibroblast treated with age inducers or rejuvenation agents. Note that the size of the bubble indicates the percent of genes that change in the expected direction between young and old. Therefore, a larger bubble indicates a greater level of agreement with increased age and a smaller bubble shows smaller agreement with age induction and, consequently, a greater agreement with cellular rejuvenation. For example, Resveratrol has a very small bubble size in the fibroblast aging score indicating gene expression changes that are highly correlated with rejuvenation. C) RNAge score of young, iPSC-derived neurons treated with age inducers.

FIGS. 7A-7C show correlation between RNAge score and cellular hallmarks of age. A) Overview of experiments to measure the cellular hallmarks in neurons and fibroblasts. B) Measurement of age associated cellular phenotypes in fibroblasts in response to treatment with DMSO (control), 5-Iodotubercin (10 μM), Alvocidib (10 μM) or Mitoxantrone (1.1 μM). C) Measurement of age associated cellular phenotypes in PSC-derived neurons in response to treatment with DMSO (control), Fludarabine (10 μM), Vinorelbine (10 μM) or AGK-2 (0.04 μM). Absolute values are shown for nuclear area and nuclear roundness. For H3K9me3, LAP2, yH2AX, p21, BAG3 and CAV1 fluorescence intensity is relative to the DMSO control. N=12. Experiment repeated 4 times each with 3 replicate wells for both neurons and fibroblasts

FIGS. 8A-8G show functions associated with aging in primary fibroblasts, FC and SN. A) PCA plot of all samples sequenced for this experiment. FC=frontal cortex, pF=primary fibroblast, SN=substantia nigra. B) Plot showing the number of overlapping functions associated with aging in FC (frontal cortex), SN (substantia nigra) and pF (fibroblasts). Down indicates that genes associated with this function are expressed at lower levels in young than old. Up indicates that genes associated with this function are expressed at higher levels in young than in old. C-G). Functional enrichment of genes associated with each of our aging signatures from FIG. 1E.

FIGS. 9A-9F show extended characterization and validation of iPSC reprograming as a model of cellular rejuvenation. A) Schema outlining the protocol used to generate iPSC-FIBs. B) Venn diagram of DEGs between iPSCs and iPSC-FIBs of young and old origin indicates lack of overlap between the DEGs in the pFIBs. C-E) Heatmaps showing differential gene expression (C), differentially methylated promoters (5mC) (D) and differences in hydroxymethylation (5hmC) (E) between young and old pFIBs (N=4 independent cell lines derived from young donors and 4 derived from old donors), iPSCs of young and old origin N=3 independent cell lines derived from young donors and 3 derived from old donors) and matched iPSC-FIBs (N=3 independent cell lines derived from young donors and 3 derived from old donors). F) Normalized expression of histone genes (HIST1H2BH, HIST1H4F, HIST3H2BB and HIST1H3E) in pFIBs (pF), iPSC-FIBs (iPS) and iPSC-FIBs (iF).

FIGS. 10A-10B show DNA methylation analyses of young, old and HGPS fibroblasts. A) PCA of promotor 5mC data from primary fibroblasts originating from young, old or individuals with HGPS. B) PCA of 5hmC data from primary fibroblasts originating from young, old or individuals with HGPS.

FIGS. 11A-11B show example immunocytochemistry images for the hallmarks of age assays performed in fibroblasts and PSC-derived neurons. A) Example immunocytochemistry images of hallmarks of aging assays in fibroblasts. B) Example immunocytochemistry images in hallmarks of aging assays performed in PSC-derived neurons. Scale 100 μM.

FIGS. 12A-12D show development of RNAge score and application to stem cell-based in vitro models. (A) Schema outlining workflow to derive, validate and apply RNAge. Cells and tissues used to derive RNAge were pF (primary fibroblasts), FC (frontal cortex) and SN (substantia nigra) young (pink) and old (brown) donors. (B) Application of RNAge to measure rejuvenation upon reprogramming of primary fibroblasts to pluripotent stem cells and differentiation to iPSC-derived fibroblasts. RNAge score is larger when old pFIB are compared to iPSC-FIB than when young donors are used. (C) Application of RNAge scoring to established experimental approaches for age induction including ectopic expression of Progerin, SLO cocktail and SATB1-KO. (D) Application of RNAge to neurons generated by transdifferentiation of primary fibroblasts (pFIB-iN) relative to neurons generated in the same manner from iPSCs (iPSCiN).

FIGS. 13A-13E show in silico screening for modulators of RNAge. (A) Schema outlining workflow to use the L1000 dataset to perform in silico drug screening for regulators of RNAge. (B) RNAge score distribution for L1000 in silico screen performed in primary fibroblasts (1HAE). (C) List of top scoring RNAge modifying compounds from (B). (D) RNAge score distribution for L1000 in silico screen performed in neurons (NEU). (E) List of top scoring RNAge modifying compounds from (D).

FIGS. 14A-14G show experimental validation of age inducing compounds identified In Silico. (A) Schema outlining how validated age inducing hits from the L1000 screen were profiled for changes in the cellular hallmarks of aging. (B) PCA of RNA-seq data from fetal fibroblasts treated with top ranked age inducers identified in the L1000 in silico screen. (C) Calculation of RNAge subscores for fibroblast treated with age inducers. (D) PCA of RNA-seq data from young PSC-derived neurons treated with top ranked age inducers identified in the L1000 in silico screen. (E) Calculation of RNAge subscores for neurons treated with age inducers. In (B-E) we performed RNAseq to measure transcript abundance and calculate RNAge rather than inferring it from the L1000 data as in FIG. 13B. (F-G) Measurement of the cellular hallmarks of aging age in fibroblasts (F) or neurons (G) in response to validated age inducers shown in (C) and (E). Absolute values are shown for nuclear area and nuclear roundness. For H3K9me3, LAP2, yH2AX, p21, BAG3 and CAV1 fluorescence intensity is relative to the DMSO control. N=12. Experiment repeated 4 times each with 3 replicate wells.

FIGS. 15A-15E show incorporation of induced aging into stem cell models of late onset disease. (A) Example immunocytochemistry images of hallmarks of aging assays in PSC-derived neurons after 4 days of treatment with Fludarabine. Scale 100 μM. (B) Quantification of the cellular hallmarks of aging in WT neurons after 4 days of treatment with Fludarabine. Example images shown in (A). Absolute values are shown for nuclear area and nuclear roundness. For H3K9me3, LAP2, yH2AX, p21, BAG3 and CAV1 fluorescence intensity is relative to the DMSO control. N=12; one sample t-test. Experiment repeated 4 times each with 3 replicate wells. (C) Presto blue viability assay in WT and APPswe/swe neurons treated with Fludarabine for 4 days relative to DMSO treatment (n=3, paired Student's t-test). (D) Total cell neuron number for WT and AD (APPswe/swe) neurons treated with DMSO or Fludarabine. Cell number normalized to the average cell number for the DMSO controls for each differentiation. (n=12). (E) Schematic depicting AD-genetic susceptibility to Fludarabine-mediated induction of cellular aging which results in genotype-specific neuronal loss.

FIGS. 16A-16G show derivation of RNAge from primary sequencing data. (A) Schematic outlining primary cell lines (fibroblasts; pFIB) and tissues (frontal cortex; F. Cortex and substantia nigra; S. nigra) sequenced in this study. Samples from young donors are in blue and old in red. (B) PCA of RNA-seq data from primary fibroblasts, frontal cortex and substantia nigra from young (blue) and old (red) donors. (C) Volcano plots showing significantly differentially expressed genes (FDR<0.1) between young and old primary tissues shown in (A-B). (D) Heatmap indicating directionality of the change in gene expression in young vs old primary tissue samples. RNA-seq datasets generated as part of this study were: Total SN (substantia nigra), Total FC (frontal cortex), Total pF (total RNA of primary fibroblast) and PolyA pF (poly-A selection of primary fibroblast). External datasets were PolyA Gage FC (poly-A selection of frontal cortex) and PolyA Gage pF (polyA selection of primary fibroblast) 6. (E) Schema outlining the steps to calculate the RNAge score. (F) Validation of the RNAge score for pF, FC, Cortex, SN, and mouse brain using external data that was not used to derive RNAge. Tissue type, species and data source are indicated on the bubble plot. (G) Application of RNAge score to young and old human primary stomach and human primary cerebellum.

FIGS. 17A-17D show reprogramming of primary fibroblasts to iPSC and generation of iPSC derived fibroblasts. (A) RNA-seq analysis showing that gene expression of fibroblast specific genes in iPSC-FIB closely resembles that of pFIB indicating the re-establishment of fibroblast identity in iPSC-FIB. (N=6 independent cell lines at each stage; 3 derived from young donors and 3 derived from old donors). (B) Volcano plots showing DEGs (FDR<0.1) between young and old pFibs, iPSCs derived from young and old donors and iPSC-FIB generated from these stem cells. (N=3 independent cell lines derived from young donors and N=3 derived from old donors). (C) PCA of RNA-seq data from primary fibroblasts originating from young, old or individuals with HGPS. (D) RNAge score of young pFIBs vs. HGPS pFIBs form this study data (top) and similar comparison using RNA-seq data from Fleischer et. al., 2018 (bottom) demonstrating that HGPS fibroblasts do not have an elevated RNAge score.

FIGS. 18A-18E show experimental validation of rejuvenating compound identified In Silico. (A) RNAge score distribution for L1000 in silico screen performed in neural progenitor cells (NPC). (B) (C) Calculation of RNAge for candidate age regulators across all concentrations available within the LINCS 1000 dataset. The RNAge score was calculated relative to every control sample within the dataset. Conditions with the most pronounced effect (yellow) were selected for downstream validation. (D) Experimental validation of mocetinostat as a regulator of RNAge in old primary fibroblasts. (E) ATAC-Seq analyses on primary young and old fibroblasts.

FIGS. 19A-19D show that selecting the optimal concentration candidate age regulators and impact hallmarks of aging. (A-B) Calculation of RNAge for every concentration of our candidate fibroblast (A) and neuron (B) age inducers available within the LINCS 1000 dataset. The RNAge score was calculated relative to every control sample within the dataset. Conditions with the most pronounced effect were selected for downstream validation. (C-D) Example immunocytochemistry images of hallmarks of aging assays in fibroblasts (C) and neurons (D) after 24 h treatment with validated age inducing compounds. Scale 100 μM.

FIG. 20 shows incorporation of induced aging into stem cell models of Parkinson's disease. Quantification of the cellular hallmarks of aging in WT dopaminergic neurons after 4 days of treatment with Fludarabine. Fludarabine induces a subset of senescence associated induced aging hallmarks in dopaminergic neurons.

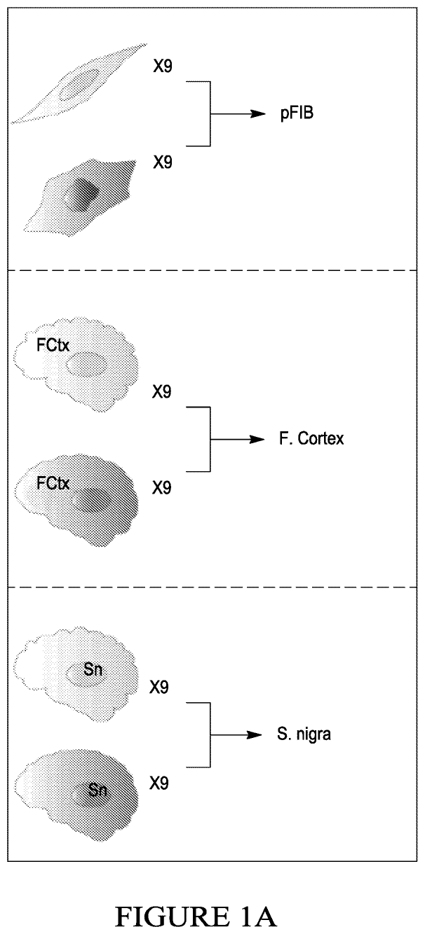

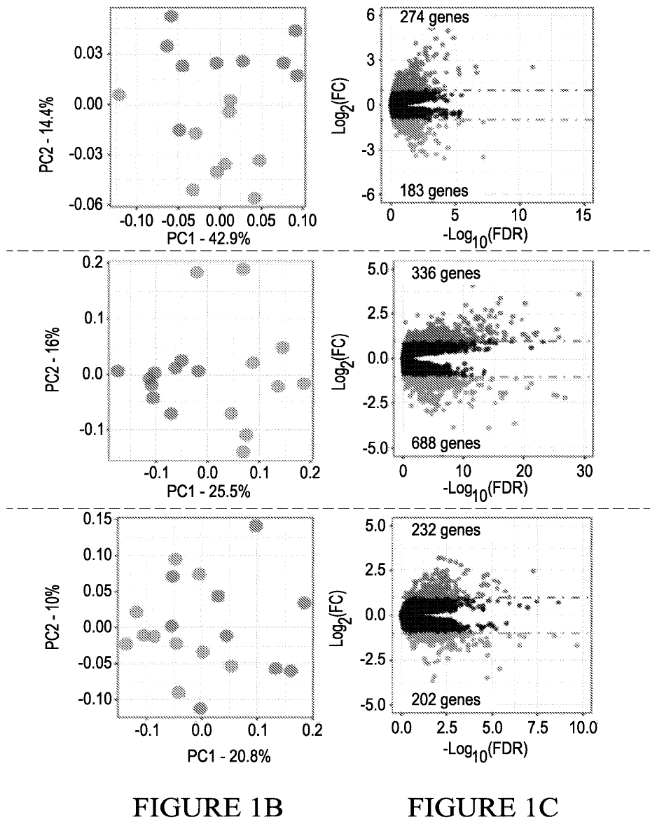

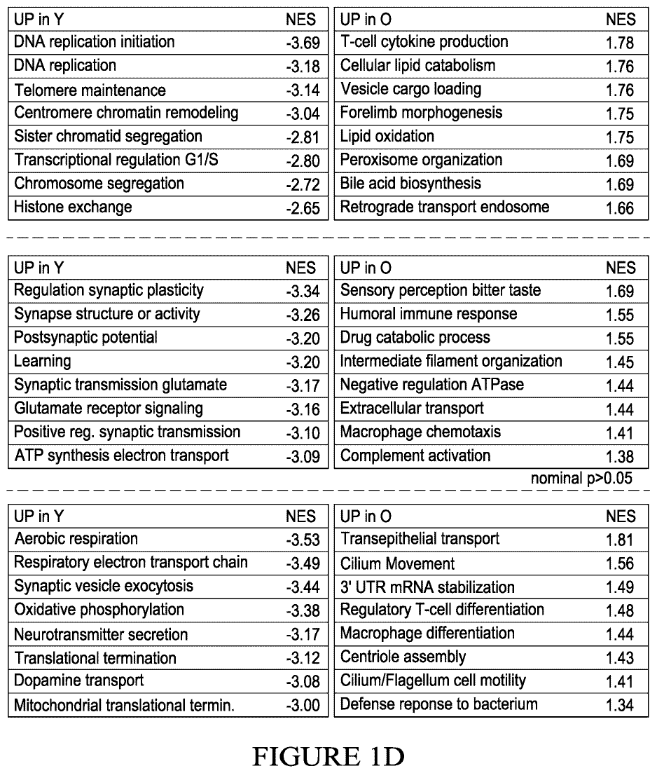

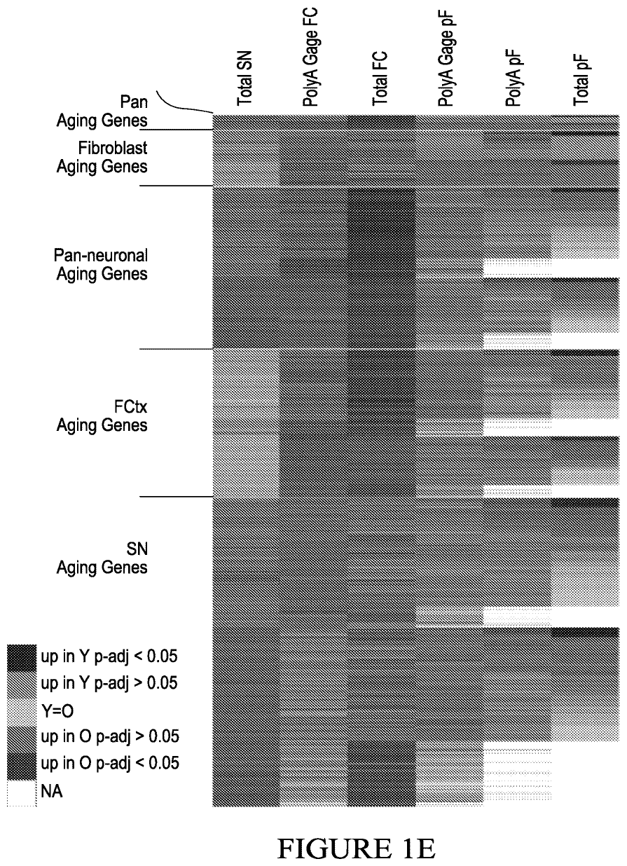

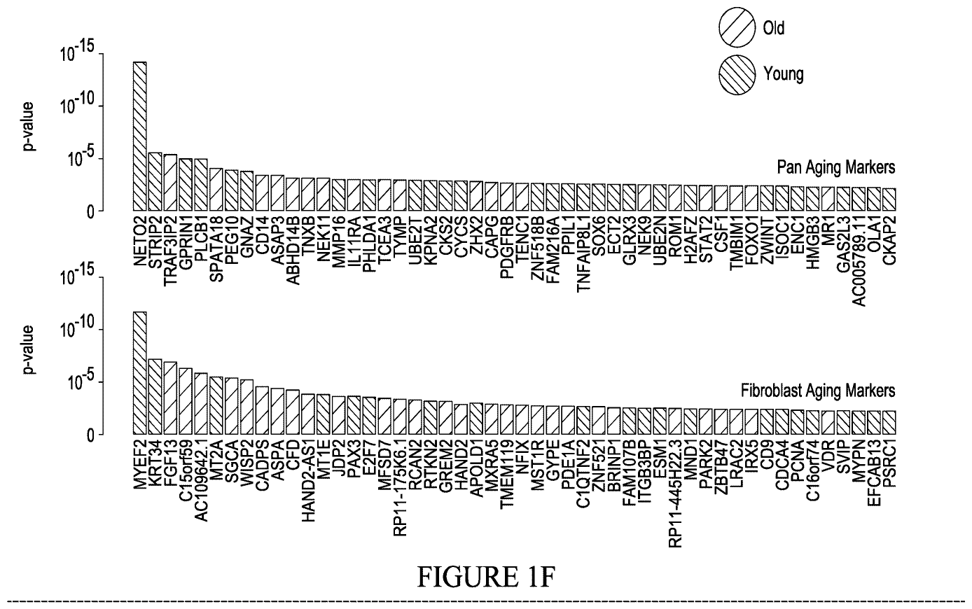

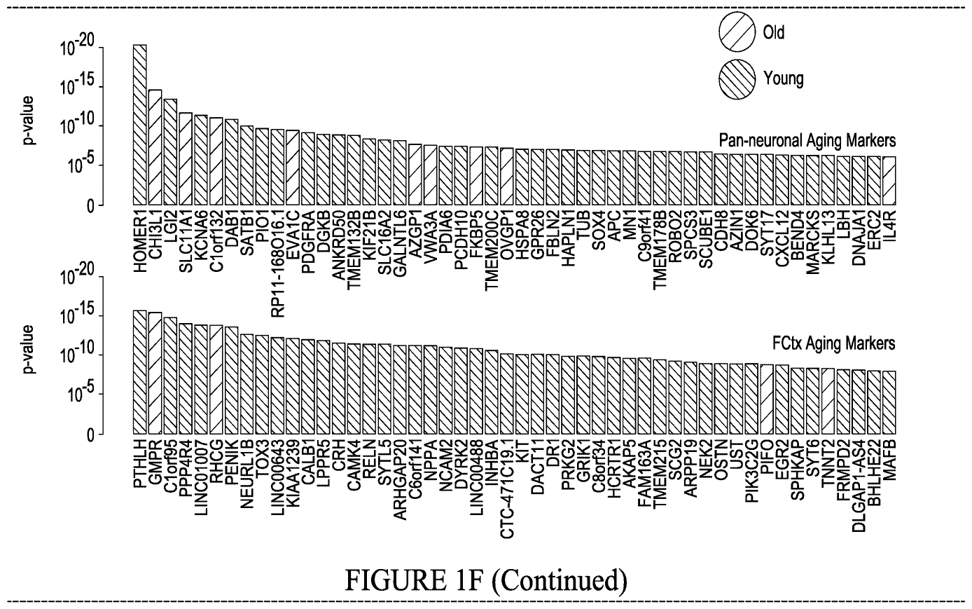

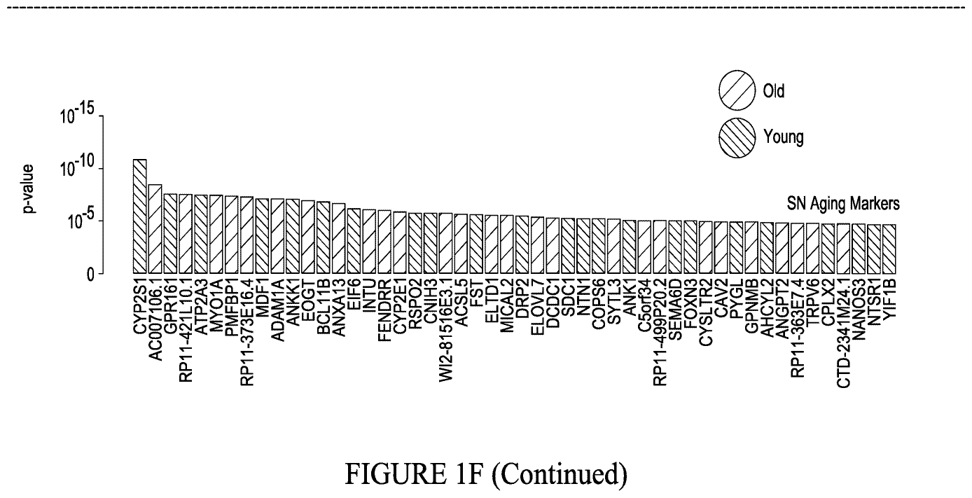

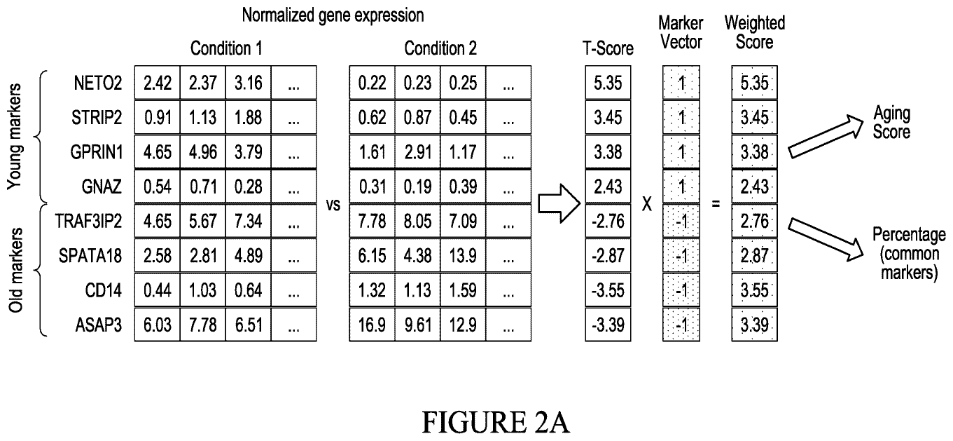

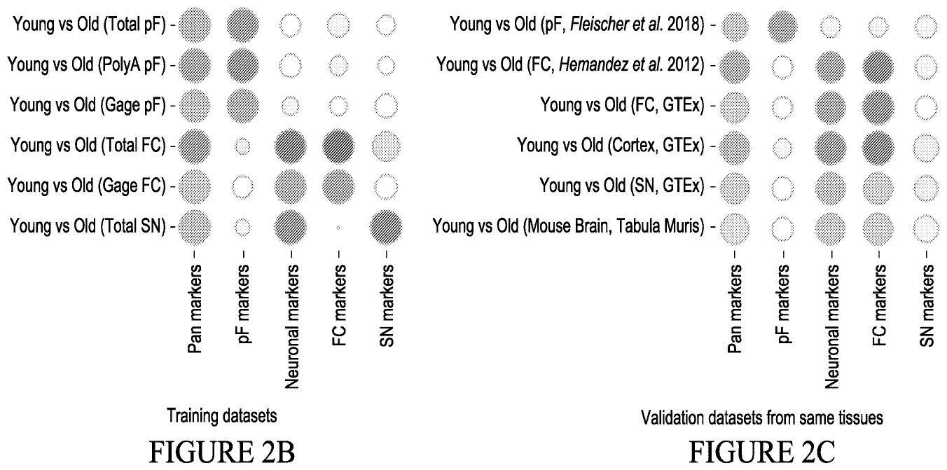

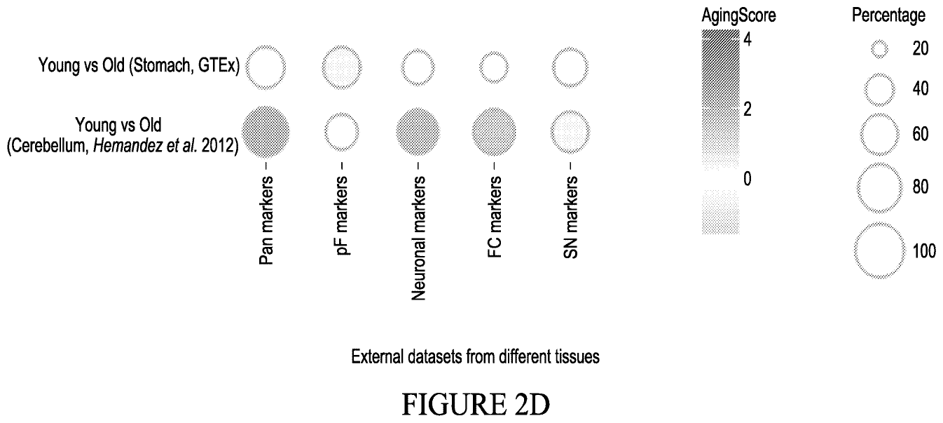

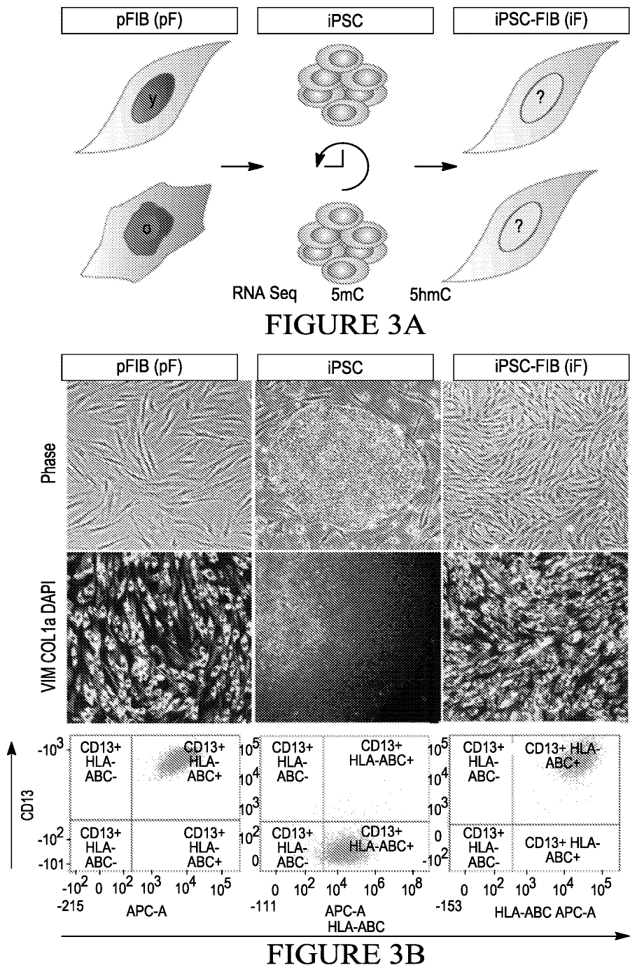

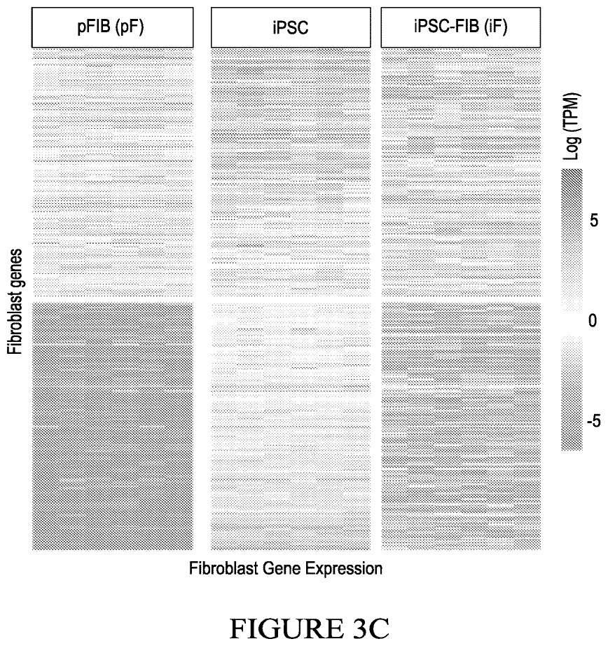

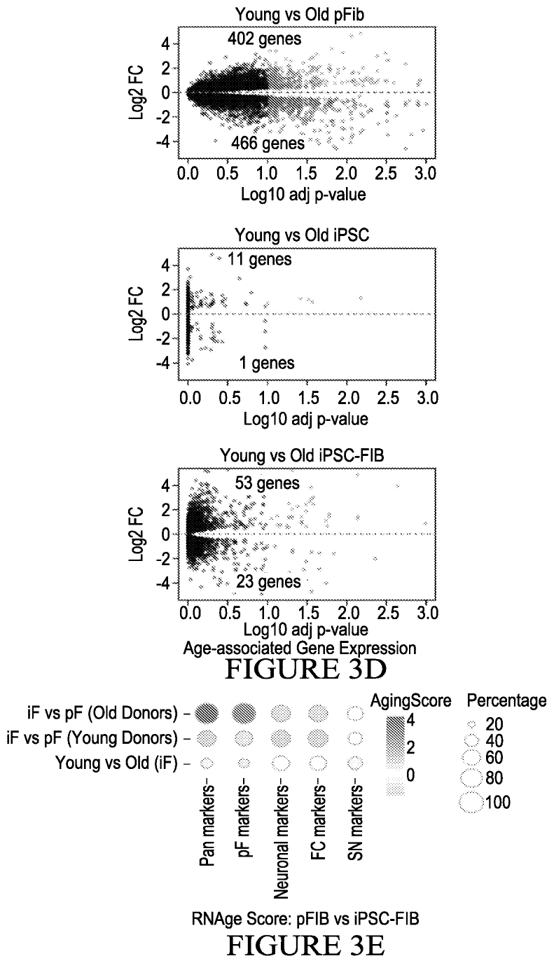

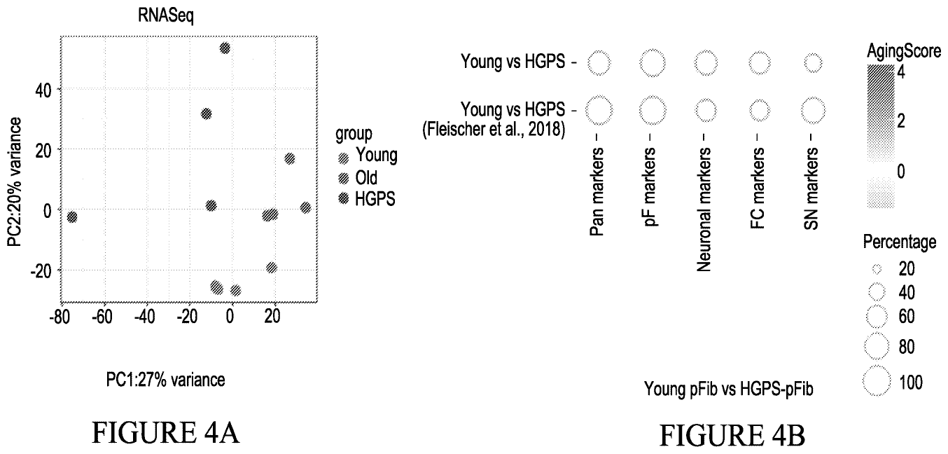

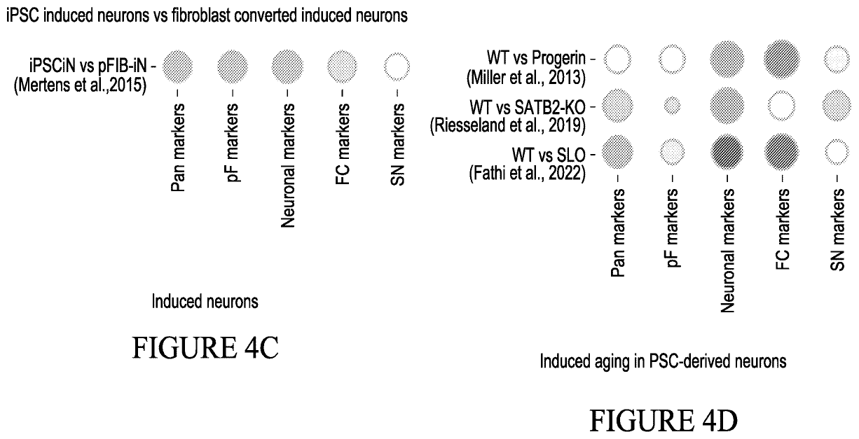

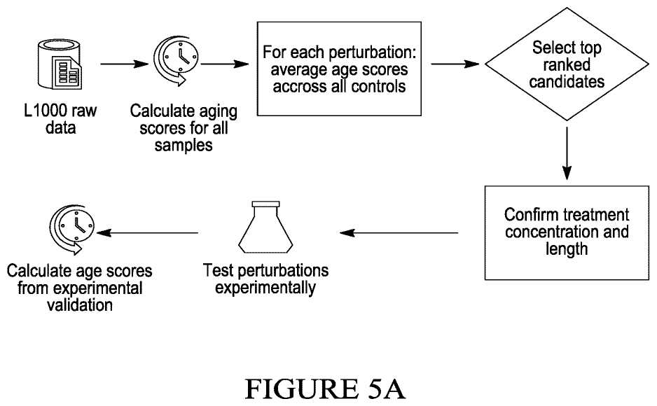

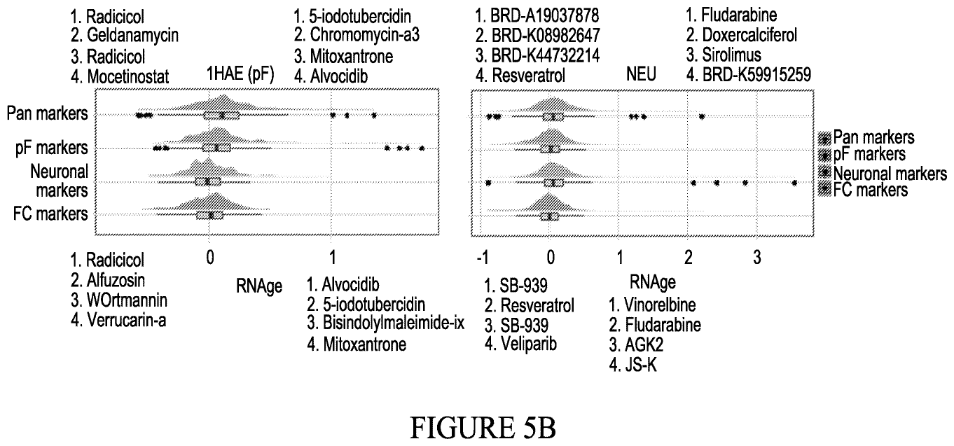

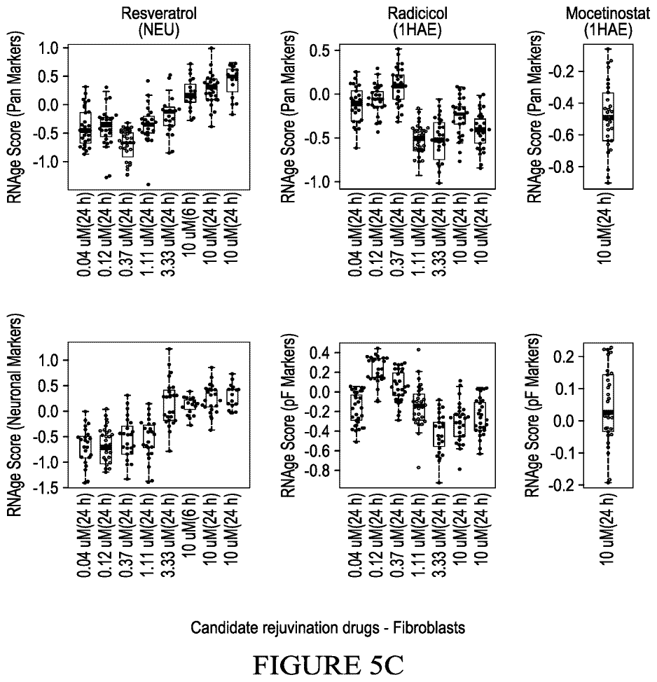

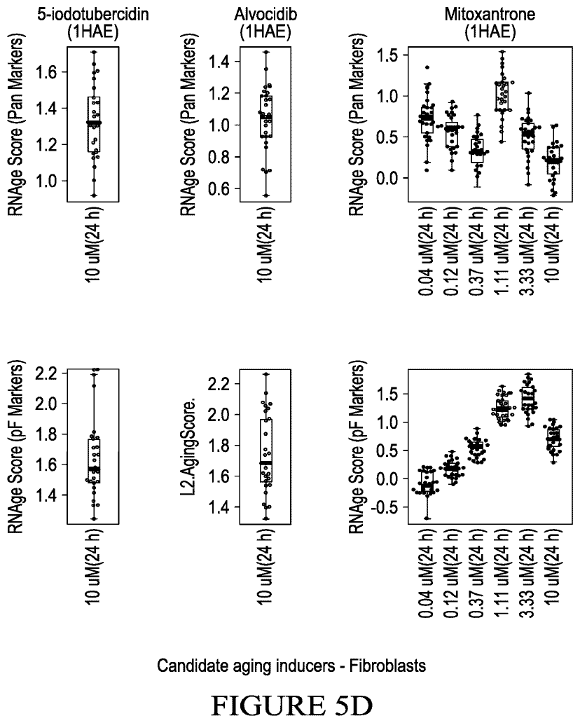

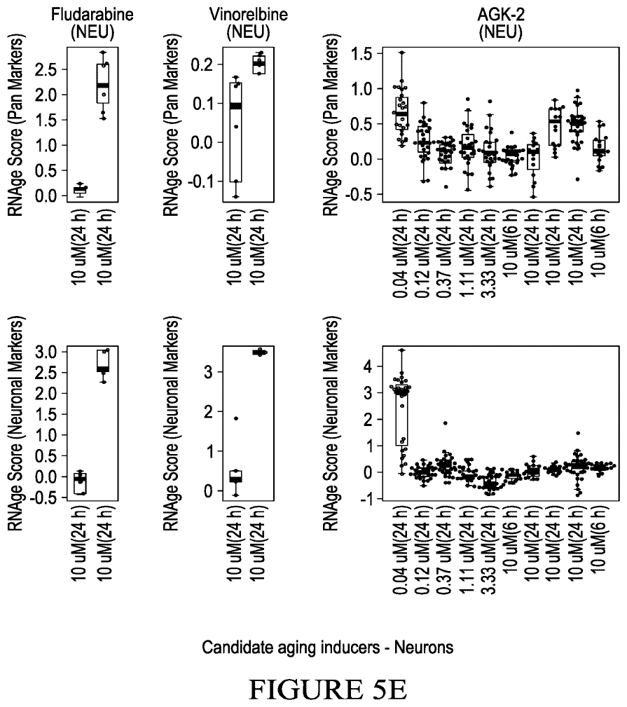

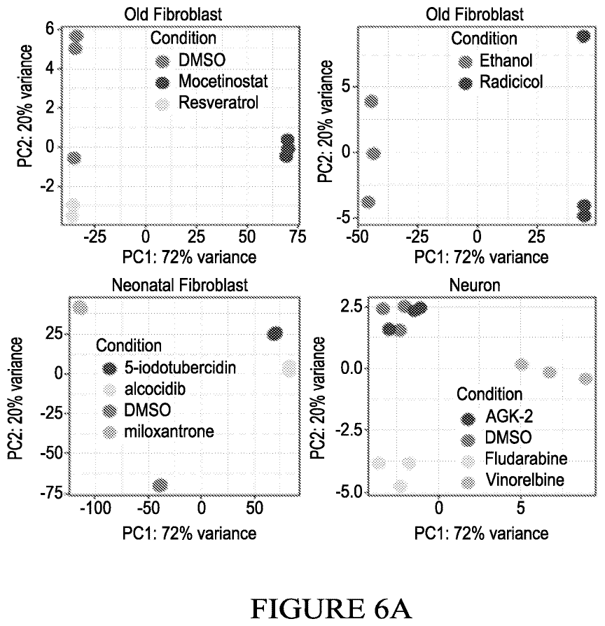

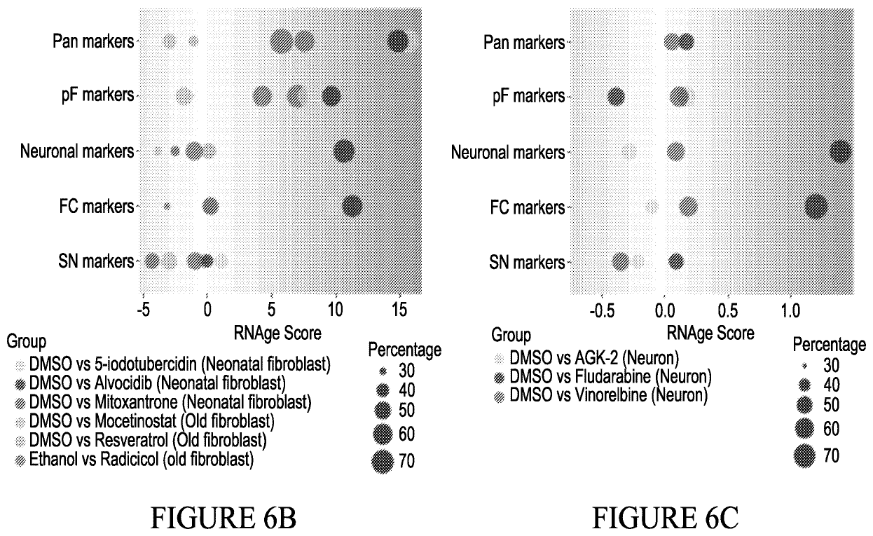

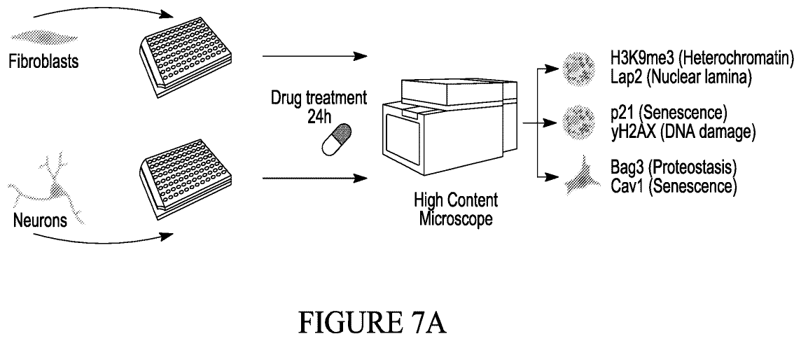

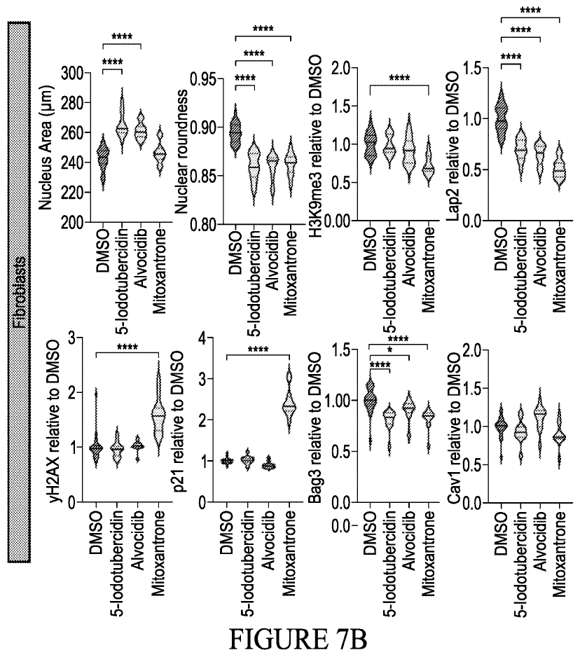

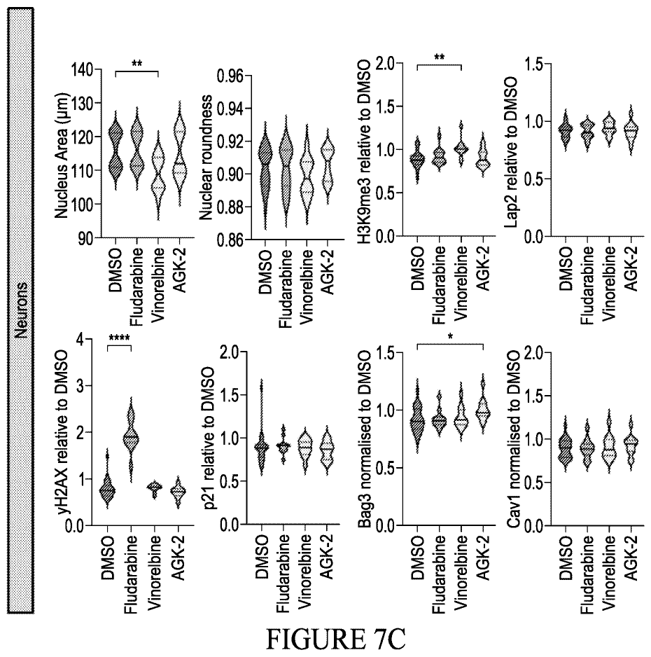

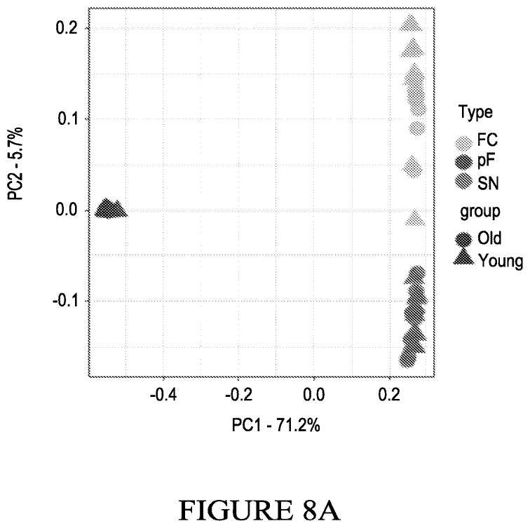

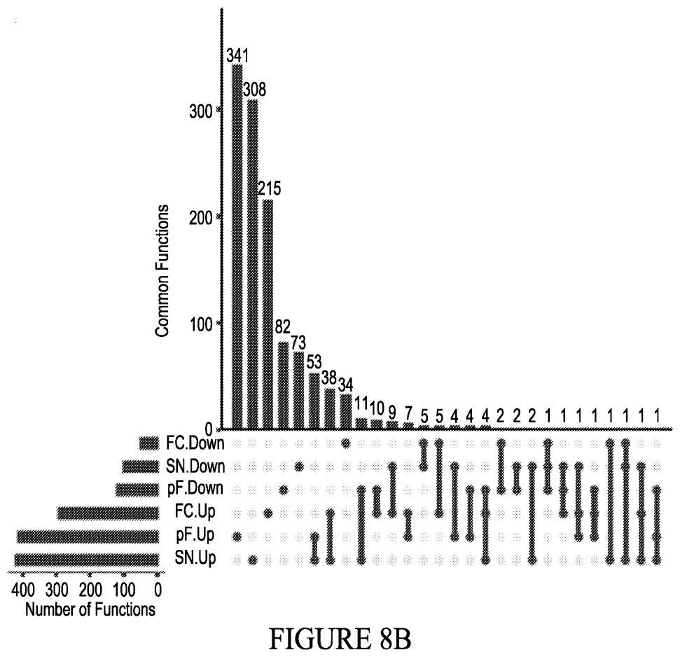

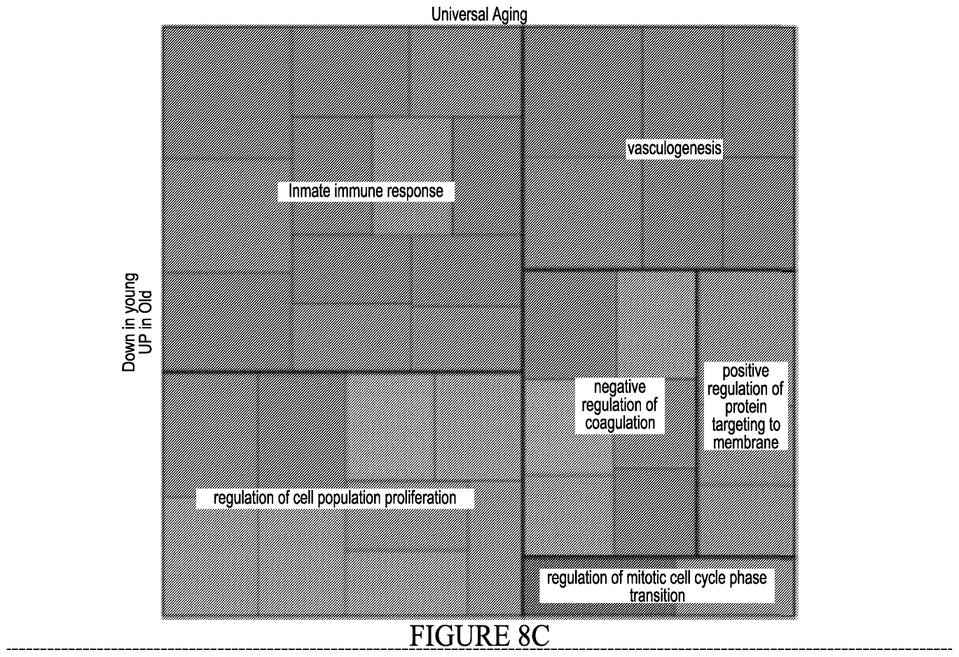

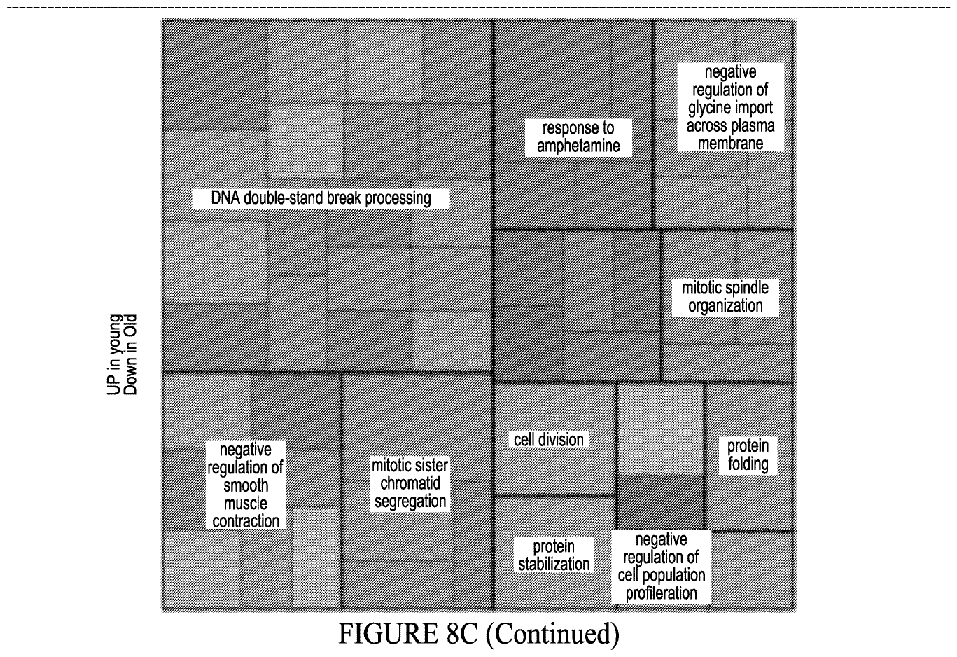

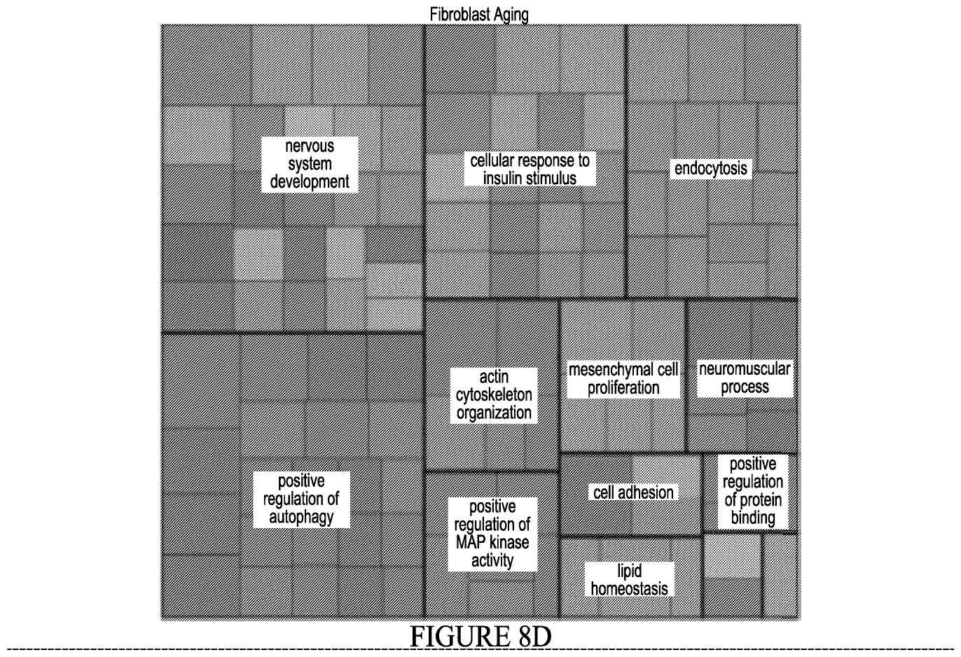

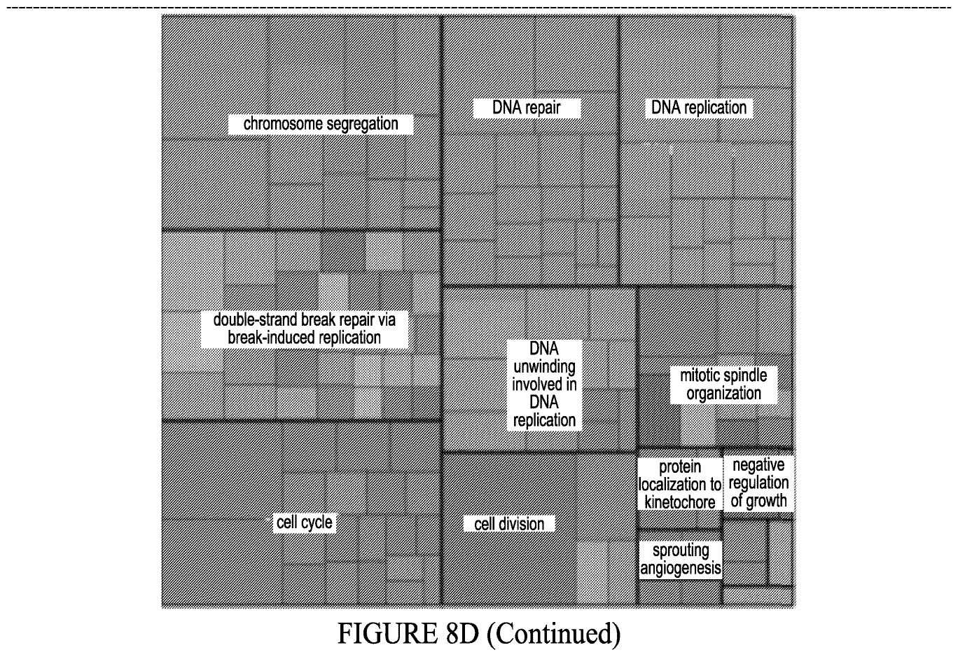

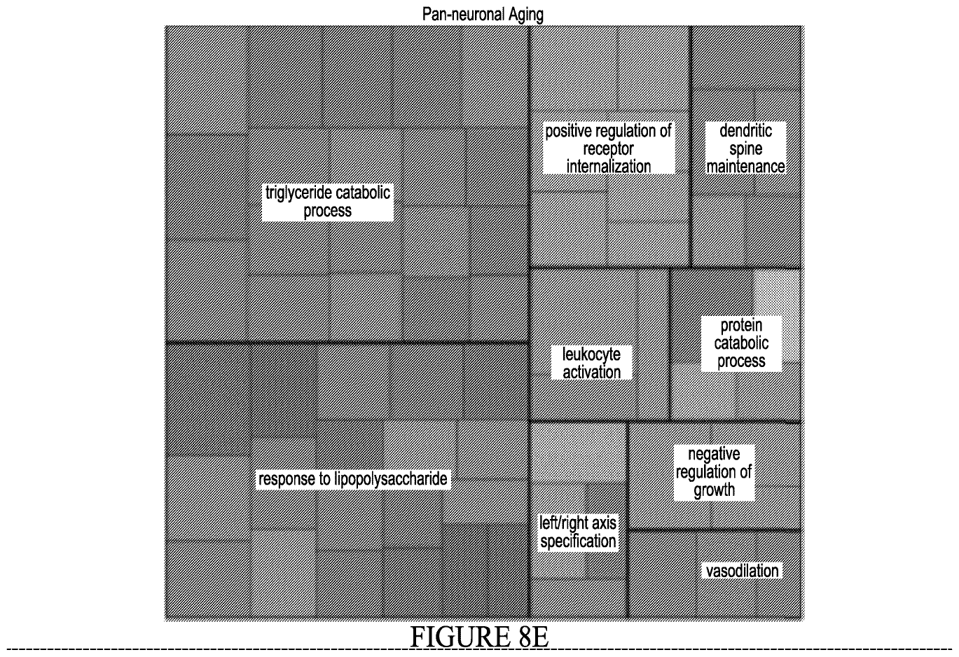

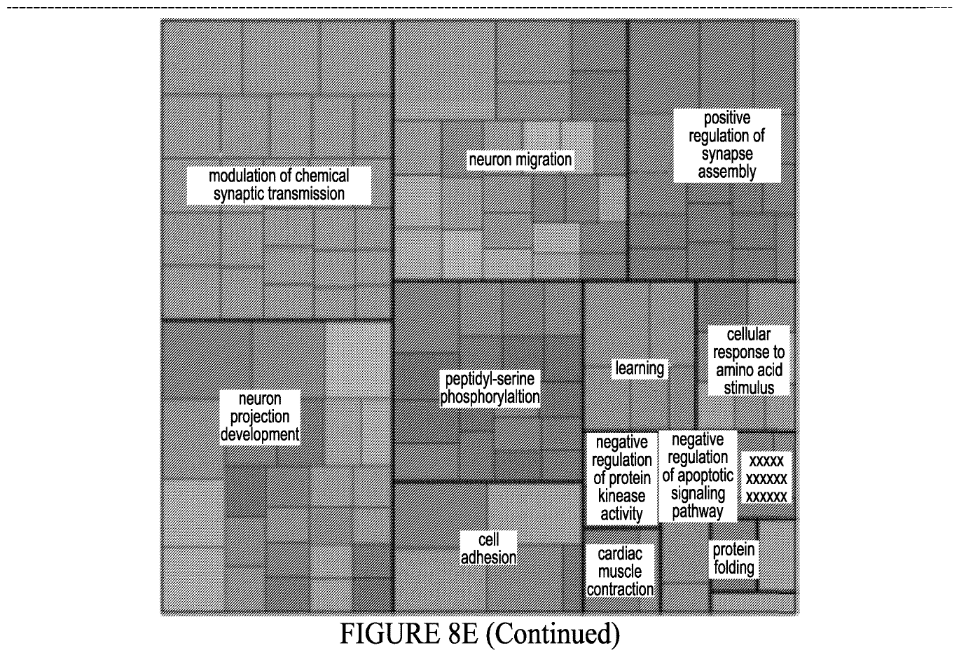

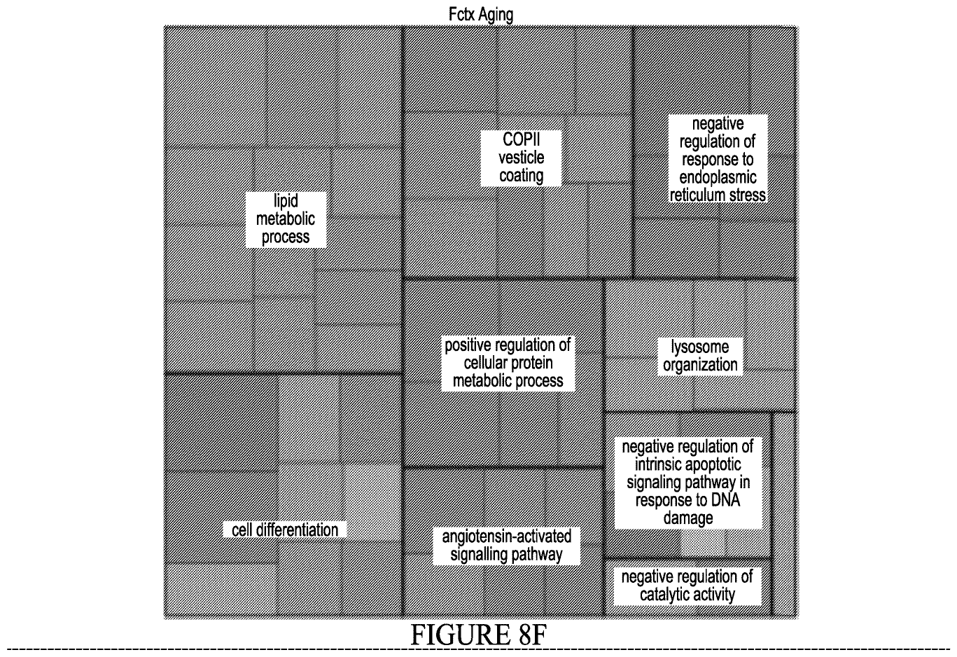

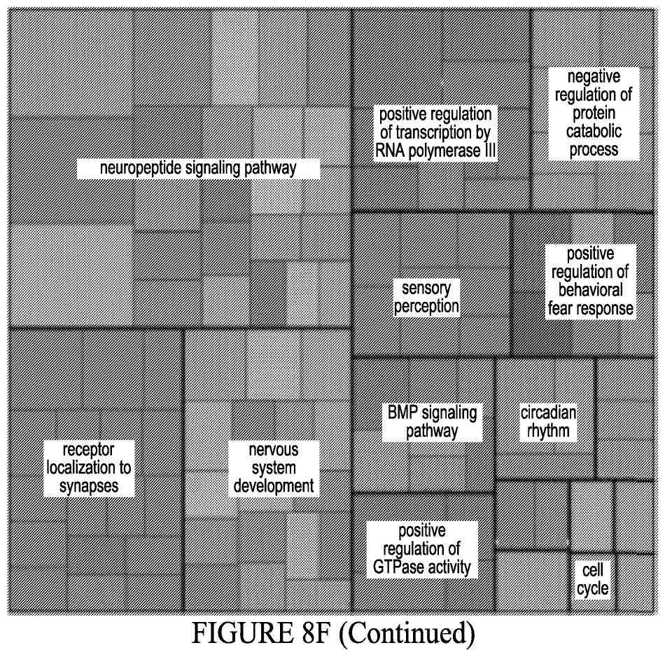

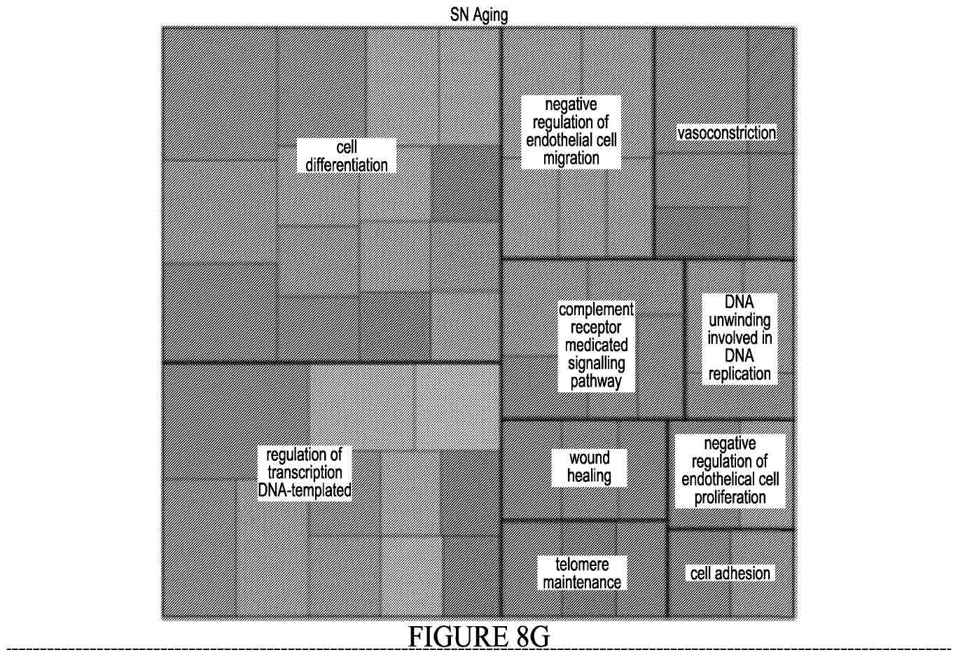

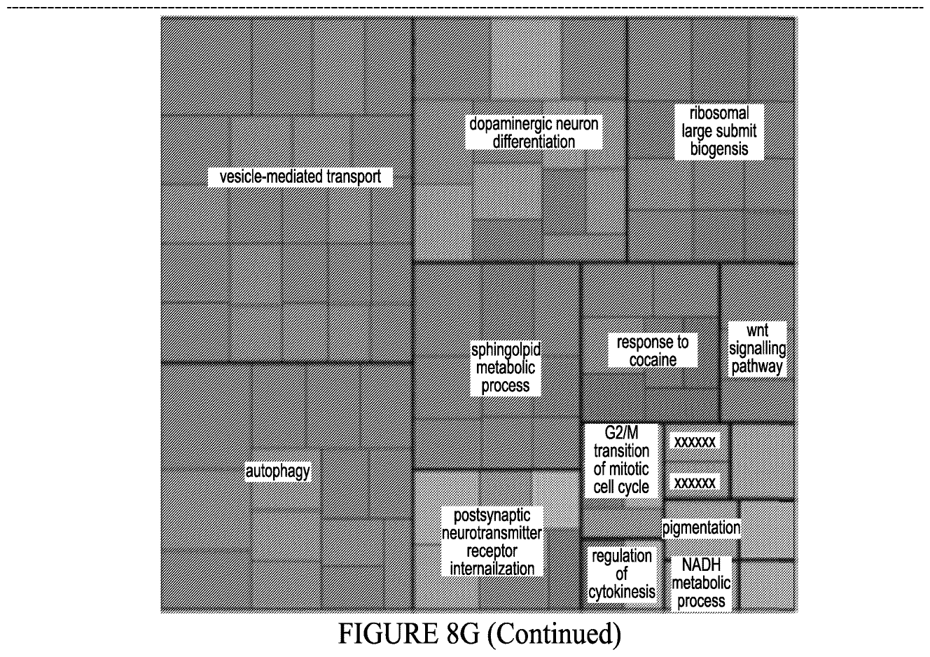

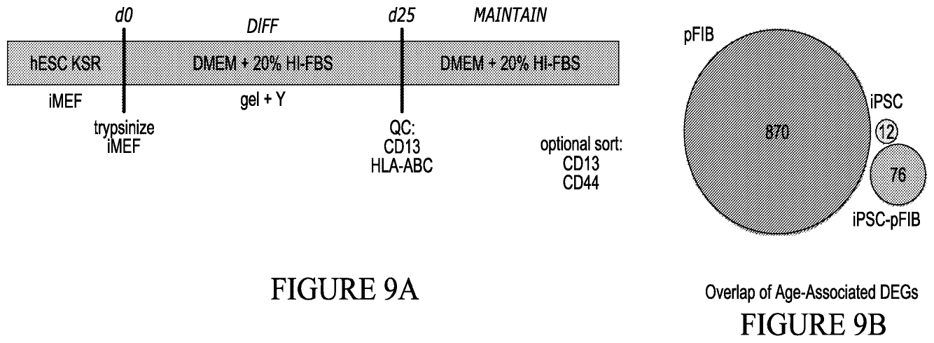

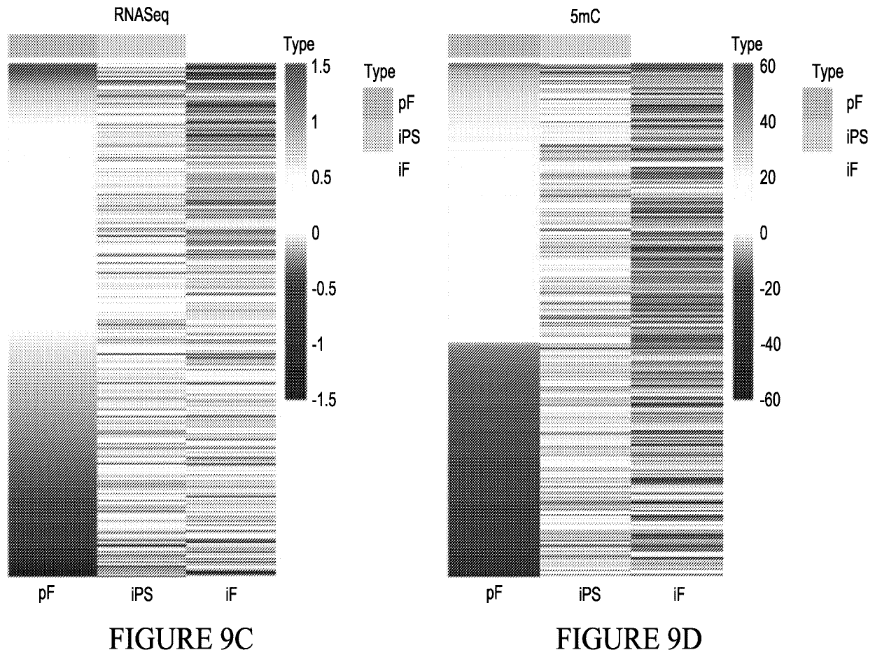

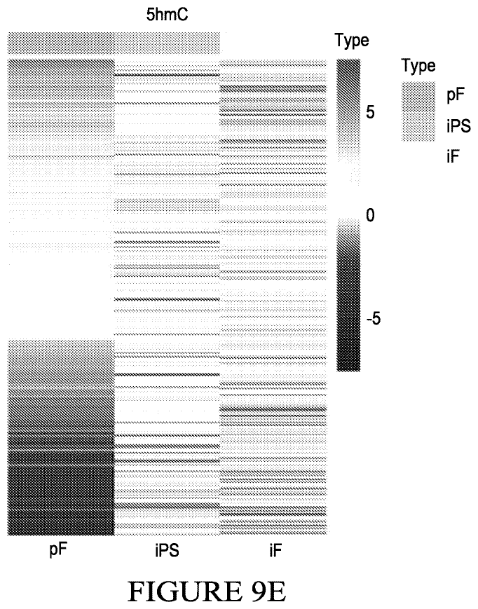

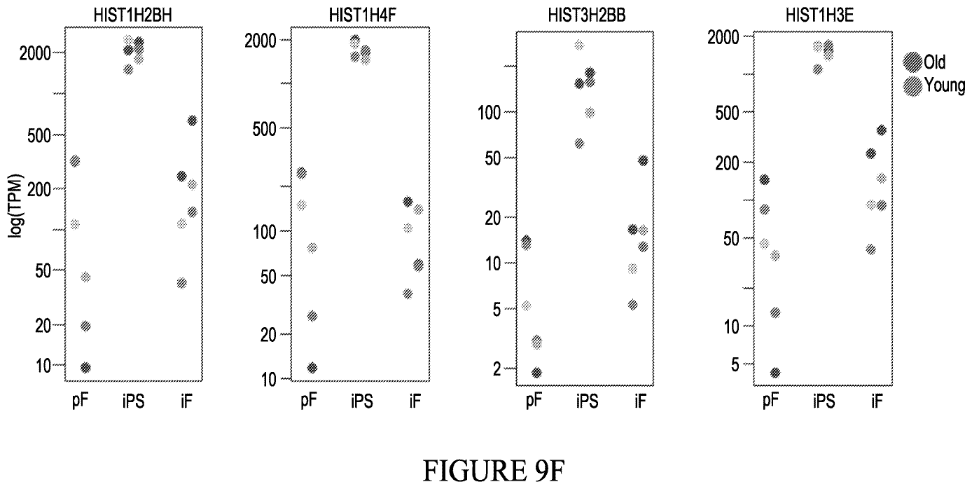

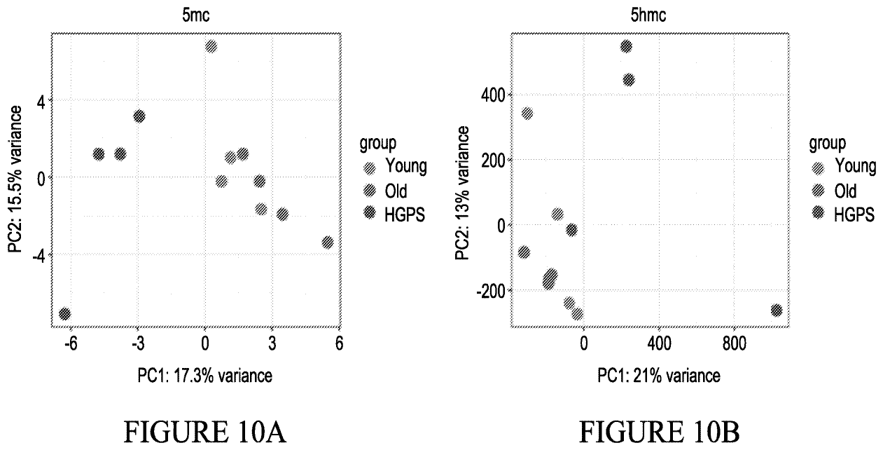

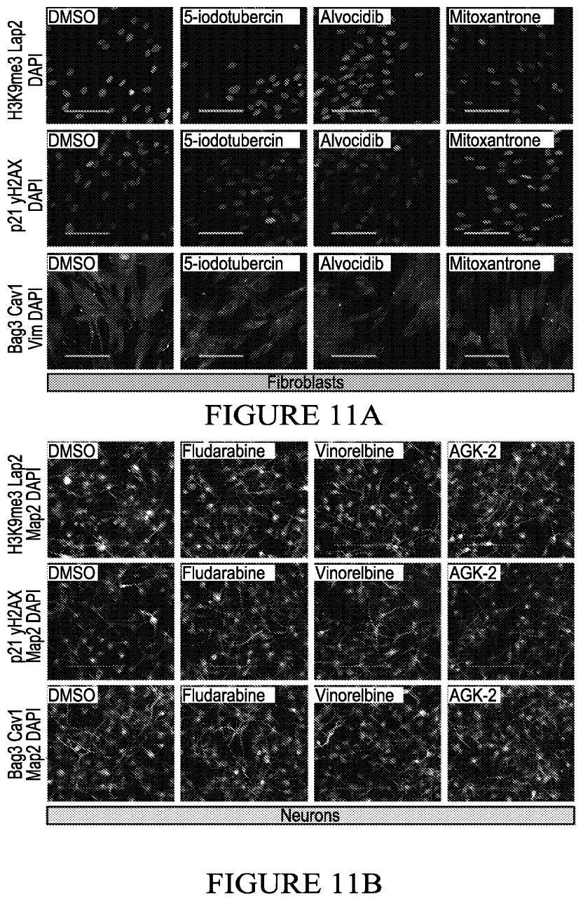

### 5. DETAILED DESCRIPTION

The present disclosure relates, in certain embodiments, to methods for modulating cellular aging and/or progression of neurodegenerative diseases (e.g., AD) in hPSC-derived cells. In certain embodiments, the methods described herein induce cellular aging or rejuvenation. In certain embodiments, the methods described herein promote progression of neurodegenerative diseases (e.g., AD). The present disclosure also relates, in certain embodiments, to cells, methods, and systems for modeling aging-related neurodegenerative diseases (e.g., AD) in vitro.

Transcriptional signatures of cellular age can be determined by comparing RNA expression in distinct tissues. The present disclosure relates, in certain embodiments, to methods of determining relative age of a biological sample by comparing RNA expression to one or more reference transcriptional signatures of cellular age. In certain embodiments, a transcriptional score is produced for a biological sample in accordance with the methods described herein. In certain embodiments, the treatment timing and/or concentration of compound is varied. In certain embodiments, the methods comprise quantifying cellular aging or rejuvenation.

The present disclosure relates, in certain embodiments, to methods of screening for novel regulators of cellular “age.” For example, in certain embodiments, a transcriptional score is produced for a biological sample following contact with one or more compounds or agents. In certain embodiments, the transcriptional score is produced by assessing one or more transcriptional signature of cellular age. In certain embodiments, one or more agent is capable of inducing a change in RNA expression of a treated biological sample is identified as a modulator of cellular age. In certain embodiments, the one or more agent elicits a shift in RNA expression of biological samples towards the transcriptional signature of “old” tissue. In certain embodiments, the one or more agents shifts RNA expression of biological samples towards the transcriptional signature of “young” samples.

For purposes of clarity of disclosure and not by way of limitation, the detailed description is divided into the following subsections:
5.1. Definitions;
5.2. RNAge Transcriptional Aging Signature;
5.3. Modulation of Cellular Age;
5.4. Method of Treatment;
5.5. Method of Screening Therapeutic Compounds;
5.6. Kits

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

An “individual” or “subject” or “patient” as described herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “young” in reference to an individual refers to an early chronological age, which for humans refers to age in years. The term “young” in reference to a cell refers to a cell displaying a marker signature of cells isolated from young donors for example, a cell state such as an immature cell, such as a young iPSC-derived somatic cell, i.e., a cell displaying a marker signature of cells isolated from young donors regardless of the age of the donor of the original primary cell that gave rise to the iPSC. This is to be contrasted with “old” iPSC-derived or indeed any somatic cell which displays a marker signature of cells isolated from old donors. An example of an old iPSC derived somatic cell is that produced when the level of genomic nucleic acid methylation in an iPSC-derived somatic cell is reduced (again, regardless of the age of the donor of the primary cell that gave rise to the iPSC) following reprogramming. A young cell may also refer to a population of “young cells” such as young primary cells derived from a donor of young chronological age as in “young primary fibroblasts.”

As used herein, the term “old” in reference to an individual refers to chronological age, which for humans refers to age in years. The term “old” in reference to a cell refers to a cell displaying a marker signature of cells isolated from old donors, for example, a cell state wherein the cell expresses one or more chronological markers associated with aged cells, or primary somatic cells from old donors. An old cell may also refer to a population of “old cells” such as old primary cells derived from a donor of old chronological age as in “old primary fibroblasts.”

As used herein, the term “donor individual” or “donor” refers to any organism, human or non-human, from which cells were obtained to provide a primary cell culture. The donor individual may be of any age, and may be non-diseased or diseased. The donor may provide cells for use in the present methods, by providing biological samples, including a biopsy, a skin biopsy, blood cells, and the like.

The term “disease,” as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent or acquired defects of the organism (as genetic or epigenetic anomalies); and/or iv) combinations of these factors.

As used herein, the term “late-onset disease” refers to a disease or medical condition of a patient manifesting as a clinical condition in middle age and old age patients. Such that a late-onset disease may include but not limited to degenerative, such as neurodegenerative diseases, such as Parkinson's disease (PD), amyotrophic lateral sclerosis, Alzheimer's, Huntington's disease, and diseases of other lineages including cardiac hypertrophy, cardiac fibrosis, Type II diabetes, age-related macular degeneration, cancers, including for example breast cancers, colon cancers, and ovarian cancers, familial adenomatous polyposis (FAP), heart disease, and the like. See, Wright et al., Trends Genet 19:97-106 (2003), incorporated by reference.

As used herein, the term “cell culture” refers to any in vitro culture of cells in an artificial medium for research or medical treatment. Included within this term are continuous cell lines (e.g., with an immortal phenotype), primary cell cultures, finite cell lines (e.g., non-transformed cells), and any other cell population maintained in vitro, including oocytes and embryos.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

The term “deficient” as used herein refers to a cell which either does not express the mRNA of a gene, a protein product of a gene, or both (i.e., devoid of such expressions), or expresses them at a reduced level.

As used herein, the term “neuronal maturation medium” or “BAGCT medium” refers to a culture medium comprising N2 medium, further comprising brain-derived neurotrophic factor (BDNF), ascorbic acid (AA), glial cell line-derived neurotrophic factor, dibutyryl cAMP and transforming growth factor type β3 for differentiating midbrain fate FOXA2/LMX1A+ dopamine (DA) neurons.

As used herein, the terms “purified,” “to purify,” “purification,” “isolated,” “to isolate,” “isolation,” and grammatical equivalents thereof as used herein, refer to the reduction in the amount of at least one contaminant from a sample. For example, a cell type is purified by at least 10%, preferably by at least 30%, more preferably by at least 50%, yet more preferably by at least 75%, and most preferably by at least 90%, reduction in the amount of undesirable cell types. Thus purification of a cell type results in “enrichment,” i.e., an increase in the amount, of the cell type in the cell culture.

As used herein, the term “differentiation agent” or “differentiation inducing compound” refers to a substance, which can be a biological molecule or a small molecule or a mixture of substances which has the property of causing a stem cell to commit to a cellular pathway leading to a somatic cell. For example, such inducing compounds may include, but are not limited to, Wnt activators or SMAD inhibitors.

As used herein, the term “sonic hedgehog protein or SHH” refers to one of three proteins in the mammalian signaling pathway family called hedgehog. SHH is believed to play a role in regulating vertebrate organogenesis, such as the growth of digits on limbs and organization of the brain. Sonic hedgehog protein is thus a morphogen that diffuses to form a concentration gradient and has different effects on the cells of the developing embryo depending on its concentration. SHH may also control cell division of adult stem cells and has been implicated in development of some cancers.

As used herein, the term “Small Mothers against Decapentaplegic” or “SMAD” are intracellular proteins that transduce extracellular signals from transforming growth factor beta ligands to the nucleus where they activate downstream gene transcription and are members of a class of signaling molecules capable of modulating directed differentiation of stem cells.

As used herein, the term “contacting” refers to exposing the cell to a compound or substance in a manner and/or location that will allow the compound or substance to exert its activity on the cell, for example, by touching the cell. Contacting may be accomplished using any suitable method and may be extracellular or intracellular. For example, in one embodiment, contacting is by introducing the compound/substance intracellularly either as such or by genetically modifying the cell, such that it expresses the compound or substance. Contacting can be achieved by a variety of methods, including exposing cells to a molecule or to a vehicle containing a molecule, delivering a polynucleotide encoding for a polypeptide to the cells through transfection. Contacting may also be accomplished by adding the compound or substance to a culture of the cells so that the contacting occurs on the outer cell membrane. Contacting may also be accomplished within a given cell by the production of a recombinant protein within a cell.

As used herein, the terms “reprogramming,” “reprogrammed” refer to the conversion of “primary cells” or “primary differentiated cells” or “primary somatic cells” into undifferentiated cells (i.e., cells that has not yet developed into a specialized cell type), such as induced pluripotent stem (iPS) cells. For example, a somatic cell culture of primary cells, (e.g., for example, primary fibroblasts isolated from donors of certain ages or primary fibroblasts isolated from patients having a disease, such as Parkinson's disease (PD), e.g., PD fibroblasts, etc.), including cell lines, may be reprogrammed into induced pluripotent stem cells. Further, an age-related marker signature appearing in the primary somatic cell culture is then altered in the reprogrammed, undifferentiated cells. In some instances, disease marker signatures appearing in the differentiated somatic cell cultures (i.e., for example, PD marker signatures) may be absent in the converted undifferentiated cells, however the exact signature may differ between iPS cells produced from different primary somatic cell donors. Primary cells may be obtained from any source, such as from donors, i.e. a biopsy, a skin biopsy, a blood draw, and the like, cell lines, and the like.

As used herein, the term “differentiated” refers to a cell, for example an unspecialized embryonic cell, that has undergone a process whereby the cell acquires the features of a specialized cell such as a heart, liver, or muscle cell. Differentiation is controlled by the interaction of a cell's genes with the physical and chemical conditions outside the cell, usually through signaling pathways involving proteins embedded in the cell surface. In certain embodiments, a “differentiated” somatic cell refers to a cell having a more committed cell type characteristic, such as a marker signature characteristic of its type. In certain embodiments, a “differentiated” iPSC-derived somatic cell refers to a cell that has at least one marker signature not present in the iPSC, for example, a marker signature of a specialized cell.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein). In certain embodiments, “inducing differentiation” refers to a process initiated by compounds that act as differentiation agents, including, but not limited to, Wnt inhibitors and/or activators, sonic hedgehog proteins and/or activators, and/or SMAD inhibitor molecules. Such agents trigger or promote the largely genetically controlled differentiation process which converts an undifferentiated cell (such as an embryonic stem cell, an induced pluripotent stem cell, a primary stem cell etc.), to a committed somatic phenotype, that of a specialized cell having a more distinct form and function, which may or may not admit further differentiation. For example, induced pluripotent stem cells may be converted into iPSC-derived fibroblasts or iPSC-derived neurons, including without limitation neuron with a specific type of junction, specific range of electrical transmission rate, specific types of neurochemical production and/or secretion, etc.

As used herein, the term “aging,” in reference to a cell or cell population, refers to any stage during the progression from expression of a young marker signature towards an old marker signature. One example of aging is the natural aging process in a cell characterized by molecular and morphological markers associated with an aged cell, such as genomic instability, telomere shortening, loss of proteostasis, loss of heterochromatin and altered gene transcription, mitochondrial dysfunction, cellular senescence, and stem cell exhaustion. Aging can also encompass maturation, whereby additional molecular, physical and functional properties of an adult cell are expressed.

As used herein, the term “rejuvenation,” in reference to a cell or cell population, refers to the progression from expression of an old marker signature to a young marker signature. In certain embodiments, rejuvenation can be achieved by contacting a cell or cell population with a “rejuvenating compound” or “rejuvenating agent” for inducing the expression of a young marker signature and/or reduces the expression of an old marker signature. In certain embodiments, rejuvenation induces a reduction in one or more molecular and morphological marker associated with an aged cell, such as genomic instability, telomere shortening, loss of proteostasis, loss of heterochromatin and altered gene transcription, mitochondrial dysfunction, cellular senescence, and stem cell exhaustion

As used herein, the term “directed differentiation” refers to a manipulation of stem cell culture conditions to induce differentiation into a particular (for example, desired) cell type, such as neuronal precursors. As used herein, the term “directed differentiation” in reference to a stem cell refers to the use of small molecules, growth factor proteins, and other growth conditions to promote the transition of a stem cell from the pluripotent state into a more mature or specialized cell fate (e.g. neuron precursors, neurons, etc.).

As used herein, the term “marker” refers to a molecular or morphologic trait characteristic of a state of a cell and therefore useful, alone or in combination with other markers, in indicating that state. A “marker” can be a “chronological marker,” which includes “age-related markers” and “maturation-related markers.” “Markers” can also be “disease related markers,” which include “late-onset disease markers.” If a single marker (or combination of markers) is sufficient in indicating the state of a cell, it constitutes a marker signature, as further explained below. In certain embodiments, a “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “age-related marker signature” refers to any chronological marker signature (comprising one or more markers) that is characteristic of the natural aging process. A single age-related marker signature may be sufficient to characterize the age of primary cells from a donor or the phenotypic stage of cells wherein an age phenotype has been induced, or a profile of a plurality of different marker signatures may be evaluated to characterize the age of primary cells from a donor or the phenotypic stage of cells wherein an age phenotype has been induced or the phenotypic age of a cell.

As used herein, the term “maturation-related marker signature” refers to any chronological marker signature that is characteristic of the natural maturation process. A single maturation-related marker signature may be sufficient to characterize the maturation stage of primary cells or the phenotypic stage of cells wherein an age phenotype has been induced, or a profile of a plurality of different marker signature maybe evaluated to characterize the maturation stage of primary cells or the phenotypic stage of cells wherein an age phenotype has been induced.

As used herein, the term “disease-related marker signature” refers to any cellular structure (molecular or morphologic) that is characteristic of a specific disease. A single marker signature may be sufficient to characterize a disease, or a profile of a plurality of different marker signatures may need to be evaluated to characterize a disease state.

As used herein, the term “cell” refers to a single cell as well as to a population of (i.e., more than one) cells. The population may be a homogeneous population comprising one cell type, such as a population of neurons or a population of undifferentiated embryonic stem cells. Alternatively, the population may comprise more than one cell type, for example a mixed neural cell population comprising neurons and glial cells. It is not meant to limit the number of cells in a population, for example, a mixed population of cells may comprise at least one differentiated cell. In one embodiment, a mixed population may comprise at least one differentiated cell and at least one stem cell. In the present disclosure, there is no limit on the number of cell types that a cell population may comprise.

As used herein, the terms “primary cell” or “primary somatic cell” refers to any cell in the body other than gametes (egg or sperm), sometimes referred to as “adult” cells, which can be reprogrammed for generating an undifferentiated iPSC in accordance with the methods disclosed herein and/or under the appropriate conditions, i.e. when contacted with a proper growth factor, compound, extracellular signal, intracellular signal, transfected with reprogramming genes (factors), etc. For example, a primary cell (culture) comprises a fibroblast cell, differentiated primary somatic cell, stem cell lines, and the like. In some embodiments, primary cells are isolated from patients. In some embodiments, primary cells are cell lines. In some embodiments, primary cells are stem cell lines. In some embodiments, primary cells are embryonic stem cells. In some embodiments, primary cells are isolated from sources such as from healthy volunteers, from patients, from patients having a particular disease or medical condition, regardless of clinical manifestation, i.e. patients having a certain genotype or phenotype. In some embodiments, primary cells are isolated from mammals. In some embodiments, primary cells are isolated from animals.

A “somatic cell” refers to any cell of an organism, which is a constituent unit of a tissue, skin, bone, blood, or organ, other than a gamete, germ cell, gametocyte, or undifferentiated stem cell. Somatic cells include progenitor cells and terminally differentiated cells. Such somatic cells include, but are not limited to, neurons, fibroblast cells, cardiomyocyte cells, epithelial cells, neuroendocrine cells, pancreatic cells, astrocytes, hematopoietic cells, midbrain dopamine neurons, motoneurons, and/or cortical neurons. As used herein, the term “neural cell culture” refers to a cell culture of neurons and/or glia wherein the cells display characteristics of cells of the central and/or peripheral nervous systems.

As used herein, the term “stem cell” refers to a cell that is totipotent or pluripotent or multipotent and is capable of differentiating into one or more different cell types, such as embryonic stem cells or stem cells isolated from organs, for example, mesenchymal or skin stem cells or induced pluripotent stem cells.

As used herein, the term “embryonic stem cell” refers to a primitive (undifferentiated) cell that is derived from preimplantation-stage embryo, embryo, placenta or umbilical cord capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “induced pluripotent stem cell” or “iPSC” refers to a type of pluripotent stem cell that is similar to an embryonic stem cell but is created when somatic (e.g., adult) cells are reprogrammed to enter an embryonic stem cell-like state by being forced to express factors important for maintaining the “stemness” of embryonic stem cells (ESCs), i.e., their ability to be led to commit to different differentiation pathways. Such factors can include certain embryonic genes (such as a OCT4, SOX2, and KLF4 transgenes) (see, for example, Takahashi and Yamanaka Cell 126, 663-676 (2006), herein incorporated by reference) which are introduced into a somatic cell.

As used herein, the term “progenitor” in reference to a cell refers to an intermediate cell stage wherein said cell is no longer a pluripotent stem cell and is also not yet a fully committed cell. Progenitor cells in this disclosure are included within somatic cells.

Stem cells according to the present disclosure can be “totipotent” stem cells, “pluripotent” stem cells, and/or “multipotent” stem cells. As used herein, the term “totipotent” refers to an ability of a cell to differentiate into any type of cell in a differentiated organism, as well as into a cell of extra embryonic materials such as placenta. As used herein, the term “pluripotent” refers to a cell or cell line that is capable of differentiating into any differentiated cell type, for example, an ability to develop into the three developmental germ layers of the organism including endoderm, mesoderm, and ectoderm. As used herein, the term “multipotent” refers to a cell or cell line that is capable of differentiating into at least two differentiated cell types.

Mouse iPSCs were reported in 2006 (Takahashi and Yamanaka, Cell 126:663-676 (2006)), and human iPSCs were reported in late 2007 (Takahashi et al. Cell. 2007 Nov. 30; 131(5): 861-72). Mouse iPSCs demonstrate important characteristics of pluripotent stem cells, including the expression of stem cell markers. Human and animal iPSCs also express stem cell markers and are capable of generating cells characteristic of all three germ layers. Unlike an embryonic stem cell, an iPSC is formed artificially by the introduction of certain embryonic genes into a somatic cell (such as an OCT4, SOX2, and KLF4 transgenes). See, for example, Takahashi and Yamanaka, Cell 126:663-676 (2006) and Agarwal et al., Nature 292-296 (2010). iPSC can be produced from adult human skin cells, or fibroblast cells, which are transfected with one or more genes such as, for example, one or more of OCT4, SOX2, NANOG, LIN28, and/or KLF4. See, Yu et al., Science 324:797-801 (2009). Alternatively, they can be produced from other types of somatic cells, such as blood or keratinocytes.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo), or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein the term, “in vitro” refers to an artificial environment and to processes or reactions that occur within an artificial environment. In vitro environments include, but are not limited to, test tubes and cell cultures.

As used herein the term, “in vivo” refers to the natural environment (e.g., in an animal) and to processes or reactions that occur within a natural environment, such as embryonic development, cell differentiation, neural tube formation, etc.

As used herein, the term “cultured cells” generally refer to cells that are maintained in vitro. Cultured cells include “cell lines” and “primary cultured cells.” The term “cell culture” refers to any in vitro culture of cells. Included within this term are continuous cell lines (e.g., with an immortal phenotype), primary cell cultures, finite cell lines (e.g., non-transformed cells), and any other cell population (notably neurons) maintained in vitro, including embryos, pluripotent stem cells.

The term “small molecule” as used herein, refers to any organic molecule of a size comparable to those organic molecules generally used in pharmaceuticals. The term excludes biological macromolecules (e.g., peptides, proteins, nucleic acids, etc.). Preferred small molecules range in size from approximately 10 Da up to about 5000 Da, more preferably up to 2000 Da, and most preferably up to about 1000 Da.

As used herein, the term “expressing” in relation to a gene or protein refers to making an mRNA or protein which can be observed using assays such as microarray assays, antibody staining assays, and the like.

### 5.2 RNAge Transcriptional Aging Signature

The present disclosure relates, in certain embodiments, to methods of determining relative age of a biological sample by comparing RNA expression to one or more reference transcriptional signatures of cellular age. Transcriptional signatures of cellular age can be determined by comparing RNA expression in distinct tissues. In certain embodiments, a transcriptional score is produced for a biological sample in accordance with the methods described herein.

The present disclosure relates, in certain embodiments, to methods of determining relative age of a biological sample by comparing RNA expression to one or more reference transcriptional signatures of cellular age. In certain embodiments, a transcriptional score is produced for a biological sample in accordance with the methods described herein.

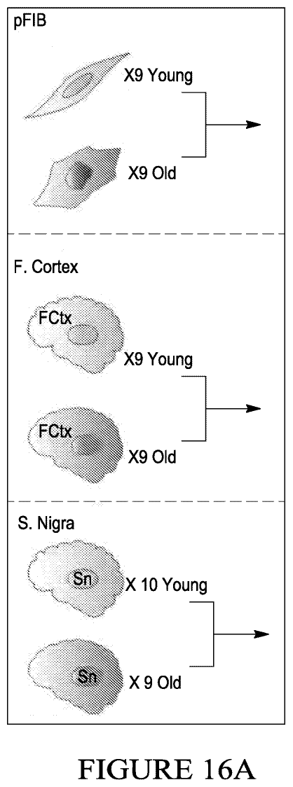

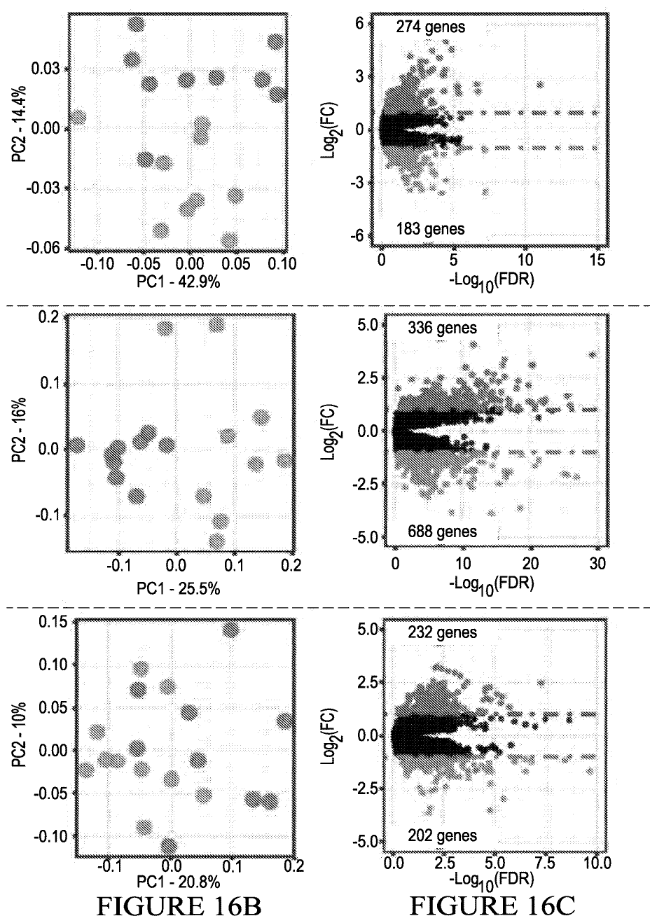

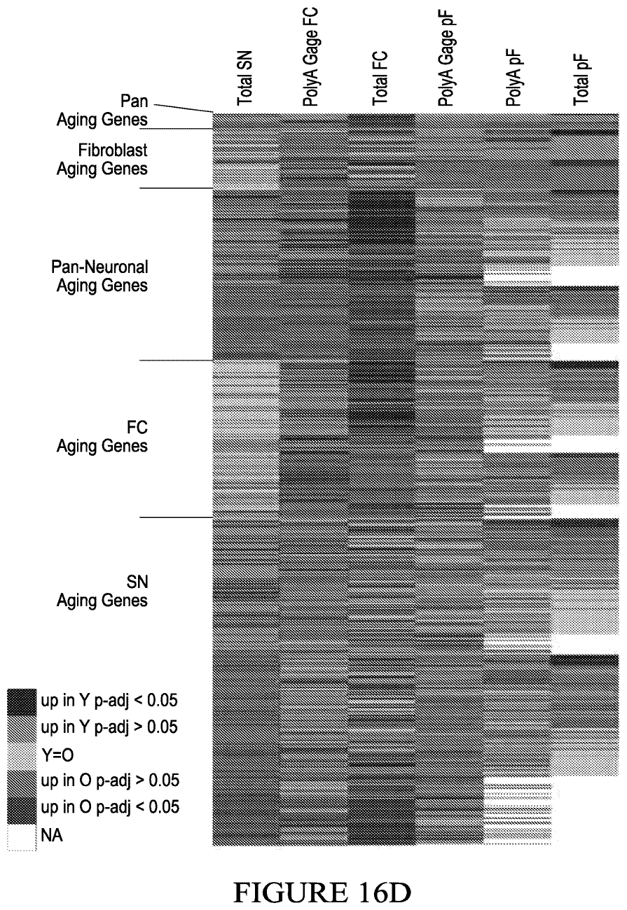

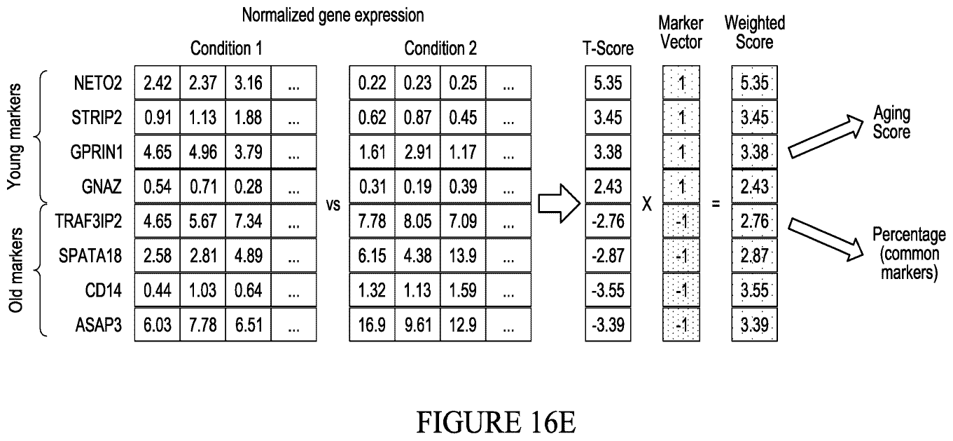

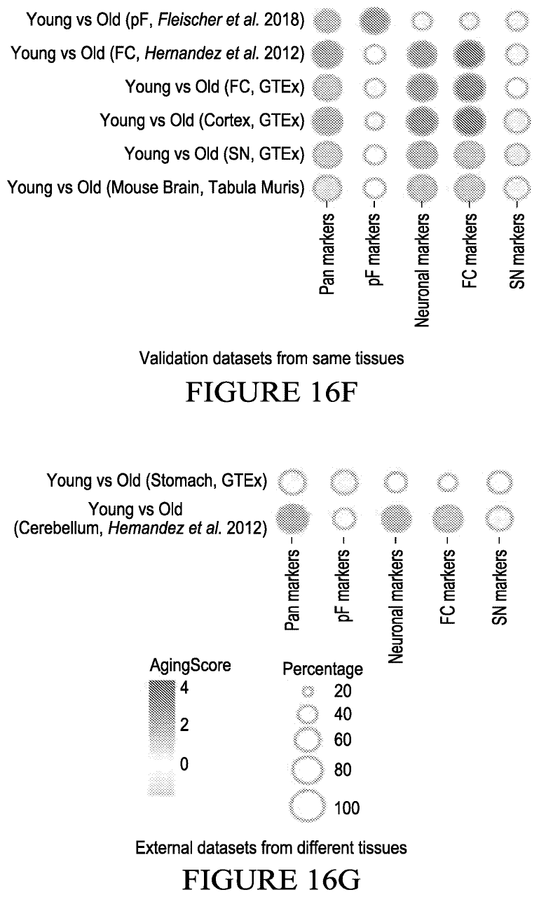

A computational pipeline (“RNAge”) was established to measure changes resulting from cellular age. To establish RNAge, sets of genes whose expression can be used to distinguish different age groups were identified. Gene sets were established by performing total RNA sequencing of young and old primary human tissue samples (fibroblasts, cortex, substantia nigra; FIG. 16A-16C). To increase robustness, external datasets were incorporated as well as internal poly-A RNA-seq data. This approach allowed the identification of five sets of mutually exclusive aging signatures: pan-aging genes that mark aging across three lineages, fibroblast-specific aging genes, pan-neuronal-specific aging genes, frontal cortex (FC)-specific aging genes and substantia nigra (SN)-specific aging genes (FIG. 16D). Markers were ranked based on the combined p-values from all training datasets for each aging gene signature panel (Pan-Aging, Fibroblast, Pan-Neuronal, FC and SN panels) individually. The top 100 ranked signatures from each panel were then used to establish scores, named ‘Pan’, ‘pF’, ‘Neuronal’, ‘FC’, and ‘SN’. The aging score is based on both the magnitude of the difference in expression between the two datasets and the directionality of the change across the top 100 age-regulated genes (see methods). The percent of genes of the age signature that change expression in the expected direction were also included to assess whether the aging score is driven by very large changes in only a small subset of the age-associated genes or by a broader change across most of the genes. RNAge performed well when validated on several independent test cases including: transcriptomic datasets from old versus young human primary fibroblasts; in both RNA-seq and microarray-based comparisons of young versus old human frontal cortex; young and old substantia nigra; and young and old mouse brain (FIG. 16F). Finally, the ‘Pan’ and ‘Neuronal’ aging scores were predictive of cellular age in the cerebellum (FIG. 16G).

### 5.3 Modulation of Cellular Age

Conventional reprogramming of somatic cells to induced pluripotent stem cells (iPSCs) resets their phenotype back to an embryonic age, and thus presents a significant hurdle for modeling late-onset disorders. In addition, stem cells collected from human subjects and somatic cells derived from such stem cells are also generally devoid of age and often also of disease markers in the case of late-onset diseases. As described herein, methods are disclosed for inducing appropriate chronological marker signatures in stem cell-derived somatic cells, including without limitation human iPSC-derived lineages, and thus generating age-appropriate cell cultures suitable as disease models.

### 5.3.1 Methods for Inducing Cellular Aging

The present disclosure relates, in certain embodiments, to methods for modulating cellular aging and/or progression of neurodegenerative diseases (e.g., Alzheimer's Disease or Parkinson's Disease). In certain embodiments, the methods described herein induce cellular aging. In certain embodiments, the methods described herein promote progression of neurodegenerative diseases. In certain embodiments, the methods comprise contacting a cell or population of cells with one or more age-inducing agent. In certain embodiments, a transcriptional score is produced for a biological sample in accordance with the methods described herein. In certain embodiments, the one or more age-inducing agent elicits a shift in RNA expression of biological samples towards the transcriptional signature of “old” tissue.

Methods of the present disclosure can also be applied to methods of quantifying cellular aging. In certain embodiments, a transcriptional score is evaluated for a biological sample that is contacted with one or more age-inducing agent. In certain embodiments, the transcriptional score is compared to one or more reference transcriptional signatures of cellular age.

In certain embodiments, contacting the cell or population of cells with one or more age-inducing agent increases the expression level or presence in the cell or population of cells of one or more chronological marker signature of an old cell. In certain embodiments, contacting the cell or population of cells with one or more age-inducing agent reduces the expression level or presence in the cell or population of cells of one or more chronological marker signatures of a young cell.

The methods of the present invention can be applied for the production of aged cells or mature cells from somatic cells (whether iPSC-derived or primary cells), from stem cells or from fully differentiated or partially differentiated cells. In certain embodiments, the cells are derived from iPSC, stem cells, embryonic stem cells, skin stem cells from adult individuals, mesenchymal stem cells, somatic cells, neurons, and fibroblasts.

The present disclosure can also be applied to induce aging of a variety of cell lineages. These cells include major cell types found in a variety tissues and organs, including, but not limited to, brain, heart, liver, kidney, spleen, muscle, skin, lung, blood, artery, eye, bone marrow, and lymphatic system. In certain embodiments, In certain embodiments, the cells are hPSC-derived cells. In certain embodiments, the cell or population of cells comprise neurons, motoneurons, cortical neurons, peripheral sensory neurons, or midbrain dopamine neurons.

In certain embodiments, the cells are iPS cell-derived fibroblasts. In certain embodiments, one or more the age-inducing agent is selected from the group consisting of one or more cyclin-dependent kinase (CDK) inhibitor, one or more adenosine kinase inhibitor, one or more topoisomerase II inhibitor, In certain embodiments, the one or more CDK inhibitor comprises alvocidib. In certain embodiments, the one or more adenosine kinase inhibitor comprises 5-iodotubercin. In certain embodiments, the one or more topoisomerase II inhibitor comprises mitoxantrone. In certain embodiments, the one or more PKC inhibitor comprises bisindolylmaleimide-ix. In certain embodiments, the one or more DNA binding agent comprises chromomycin-a3. In certain embodiments, the one or more age-inducing agent is selected from the group consisting of one or more DNA synthesis inhibitor, one or more microtubule antagonist, one or more Sirtuin-2 (SIRT2) inhibitor, BRD-K25731886, doxercalciferol, and combinations thereof. In certain embodiments, the one or more DNA synthesis inhibitor comprises fludarabine. In certain embodiments, the one or more microtubule antagonist comprises vinorelbine. In certain embodiments, the one or more SIRT2 inhibitor comprises AGK2.

In certain embodiments, the treatment timing and/or concentration of the one or more age-inducing agent is varied. In certain embodiments, the one or more age-inducing agent is administered at a concentration of between about 0.01 and 5 μM, or any value in between, for example, between about 0.02 and 5 μM, or between about 0.03 and 5 μM, or between about 0.4 and 5 μM, or between about 0.05 and 5 μM, or between about 0.1 and 5 μM, or between about 0.5 and 5 μM, or between about 1 and 5 μM, or between about 2 and 5 μM, or between about 3 and 5 μM, or between about 4 and 5 M, or between about 0.01 and 4 μM, or between about 0.01 and 3 μM, or between about 0.01 and 2 μM, or between about 0.01 and 1 μM, or between about 0.01 and 0.5 μM, or between about 0.01 and 0.2 μM, or between about 0.01 and 0.1 μM, or between about 0.01 and 0.05 μM. In certain embodiments, the one or more age-inducing agent is administered at a concentration of between about 5 and 50 μM, or any values in between, for example between about 10 and 50 μM, or between about 15 and 50 μM, or between about 20 and 50 μM, or between about 30 and 50 μM, or between about 40 and 50 μM, or between about 5 and 40 μM, or between about 5 and 30 μM, or between about 5 and 20 μM, or between about 5 and 15 μM, or between about 5 and 10 μM. In certain embodiments, the one or more age-inducing agent is administered at a concentration of about 0.01 μM, about 0.02 μM, about 0.03 μM, about 0.04 μM, about 0.05 μM, about 0.06 μM, about 0.07 μM, about 0.08 μM, about 0.09 UM, about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, or about 50 μM.

In certain embodiments, the one or more age-inducing agent described herein is contacted to a cell for at least about 1 day, at least about 2 days, at least about 3 days, at least about 4 days, at least about 5 days, at least about 6 days, at least about 7 days, at least about 8 days, at least about 9 days, at least about 10 days. In certain embodiments, the one or more age-inducing agent is contacted to the cells for up to about 1 day, up to abut 2 days, up to about 3 days, up to about 4 days, up to about 5 days, up to about 6 days, up to about 7 days, up to about 8 days, up to about 9 days, up to about 10 days. In certain embodiments, the one or more age-inducing agent is contacted to the cells for about 1 day, about 2 days, about 3 days, about 4 days, about 5 days, about 6 days, about 7 days, about 8 days, about 9 days, or about 10 days.

The present disclosure provides methods for inducing cellular aging in a cell or population of cells, comprising contacting the cell or population of cells with one or more age-inducing agent. In certain embodiments, the cell or population of cells comprise fibroblasts. In certain embodiments, the one or more age-inducing agent is selected from the group consisting of one or more cyclin-dependent kinase (CDK) inhibitor, one or more adenosine kinase inhibitor, one or more topoisomerase II inhibitor, one or more protein kinase C (PKC) inhibitor, one or more DNA binding agent, and combinations thereof. In certain embodiments, the one or more CDK inhibitor comprises alvocidib. In certain embodiments, the one or more adenosine kinase inhibitor comprises 5-iodotubercin. In certain embodiments, the one or more topoisomerase II inhibitor comprises mitoxantrone. In certain embodiments, the one or more PKC inhibitor comprises bisindolylmaleimide-ix. In certain embodiments, the one or more DNA binding agent comprises chromomycin-a3. In certain embodiments, the cell or population of cells comprise neurons, motoneurons, cortical neurons, peripheral sensory neurons, or midbrain dopamine neurons. In certain embodiments, the one or more age-inducing agent is selected from the group consisting of one or more DNA synthesis inhibitor, one or more microtubule antagonist, one or more Sirtuin-2 (SIRT2) inhibitor, BRD-K25731886, doxercalciferol, and combinations thereof. In certain embodiments, the one or more DNA synthesis inhibitor comprises fludarabine. In certain embodiments, the one or more microtubule antagonist comprises vinorelbine. In certain embodiments, the one or more SIRT2 inhibitor comprises AGK2. In certain embodiments, contacting the cell or population of cells with one or more age-inducing agent increases the expression level or presence in the cell or population of cells of one or more chronological marker signature of an old cell. In certain embodiments, contacting the cell or population of cells with one or more age-inducing agent decreases the expression level or presence in the cell or population of cells of one or more chronological marker signature of a young cell. In certain embodiments, contacting the cell or population of cells with one or more age-inducing agent induces loss of heterochromatin, disrupted nuclear lamina, increased DNA damage, increased cellular senescence, disrupted proteostasis and increased dependence on autophagy, increased caveolin-1 (CAV1) expression, or a combination thereof. In certain embodiments, the cell or population of cells are derived from primary cells, fetal cells, pluripotent stem cells, embryonic stem cells, induced pluripotent stem cells, or combinations thereof. In certain embodiments, the cell or population of cells are human.

The present disclosure provides an age-induced cell, wherein the age-induced cell is produced by a method comprising contacting a cell with one or more age-inducing agent. In certain embodiments, the age-induced cell is derived from fibroblasts. In certain embodiments, the one or more age-inducing agent is selected from the group consisting of one or more cyclin-dependent kinase (CDK) inhibitor, one or more adenosine kinase inhibitor, one or more topoisomerase II inhibitor, one or more protein kinase C (PKC) inhibitor, one or more DNA binding agent, and combinations thereof. In certain embodiments, the one or more CDK inhibitor comprises alvocidib. In certain embodiments, the one or more adenosine kinase inhibitor comprises 5-iodotubercin. In certain embodiments, the one or more topoisomerase II inhibitor comprises mitoxantrone. In certain embodiments, the one or more PKC inhibitor comprises bisindolylmaleimide-ix. In certain embodiments, the one or more DNA binding agent comprises chromomycin-a3. In certain embodiments, the age-induced cell is derived from neurons, motoneurons, cortical neurons, peripheral sensory neurons, or midbrain dopamine neurons. In certain embodiments, the one or more age-inducing agent is selected from the group consisting of one or more DNA synthesis inhibitor, one or more microtubule antagonist, one or more Sirtuin-2 (SIRT2) inhibitor, BRD-K25731886, doxercalciferol, and combinations thereof. In certain embodiments, the one or more DNA synthesis inhibitor comprises fludarabine. In certain embodiments, the one or more microtubule antagonist comprises vinorelbine. In certain embodiments, the one or more SIRT2 inhibitor comprises AGK2. In certain embodiments, the age-induced cell is derived from primary cells, fetal cells, pluripotent stem cells, embryonic stem cells, induced pluripotent stem cells, or combinations thereof. In certain embodiments, the age-induced cell is human. In certain embodiments, the cell with one or more age-inducing agent increases the expression level or presence of one or more chronological marker signature of an old cell. In certain embodiments, contacting the cell with one or more age-inducing agent decreases the expression level or presence of one or more chronological marker signature of a young cell. In certain embodiments, contacting the cell with one or more age-inducing agent induces loss of heterochromatin, disrupted nuclear lamina, increased DNA damage, increased cellular senescence, disrupted proteostasis and increased dependence on autophagy, increased caveolin-1 (CAV1) expression, or a combination thereof.

### 5.3.2 Methods for Reducing Aging

The present disclosure provides methods for rejuvenating or reducing aging of a cell or population of cells, for example, an iPSC-derived cell, such as an iPSC-derived somatic cell (e.g., iPSC-derived fibroblasts and iPS cell-derived neurons). In certain embodiments, the methods described herein induce cellular rejuvenation. The methods include contacting the cell or population of cells with one or more rejuvenating agent. In certain embodiments, a transcriptional score is produced for a biological sample in accordance with the methods described herein. In certain embodiments, the one or more rejuvenating agent elicits a shift in RNA expression of biological samples towards the transcriptional signature of “young” tissue.

Methods of the present disclosure can also be applied to methods of quantifying cellular rejuvenation. In certain embodiments, a transcriptional score is evaluated for a biological sample that is contacted with one or more rejuvenating agent. In certain embodiments, the transcriptional score is compared to one or more reference transcriptional signatures of cellular age.

In certain embodiments, contacting the cell or population of cells with one or more rejuvenating agent increases the expression level or presence in the cell or population of cells of one or more chronological marker signatures of a young cell. In certain embodiments, contacting the cell or population of cells with one or more rejuvenating agent reducing the expression level or presence in the cell or population of cells of one or more chronological marker signature of an old cell.

The methods of the present invention can be applied for the production of young cells from somatic cells (whether iPSC-derived or primary cells), from stem cells or from fully differentiated or partially differentiated cells. In certain embodiments, the cells are derived from iPSC, stem cells, embryonic stem cells, skin stem cells from adult individuals, mesenchymal stem cells, somatic cells, neurons, and fibroblasts. In certain embodiments, In certain embodiments, the cells are hPSC-derived cells.

In certain embodiments, the one or more rejuvenating agent comprises one or more histone deacetylase (HDAC) inhibitor. HDAC inhibitors function by inhibiting histone deacetylases, which catalyze the removal of acetyl groups from histones. Non-limiting examples of HDAC inhibitors include mocetinostat, SB-939, and BRD-A19037878.

In certain embodiments, the cell or population of cells comprise fibroblasts. In certain embodiments, the one or more rejuvenating agent is selected from the group consisting of one or more histone deacetylase (HDAC) inhibitor, one or more heat shock protein (HSP) inhibitor, one or more adrenergic receptor antagonist, one or more phosphoinositide 3-kinase (PI3K) inhibitor, and combinations thereof. In certain embodiments, the one or more HDAC inhibitor comprises mocetinostat. In certain embodiments, the one or more HSP inhibitor comprises radicicol, geldanamycin, or a combination thereof. In certain embodiments, the one or more adrenergic receptor antagonist comprises alfuzosin. In certain embodiments, the one or more PI3K inhibitor comprises wortmannin. In certain embodiments, the cell or population of cells comprise neurons, motoneurons, cortical neurons, peripheral sensory neurons, or midbrain dopamine neurons. In certain embodiments, the one or more rejuvenating agent is selected from the group consisting of one or more Sirtuin (SIRT) activator, one or more HDAC inhibitor, one or more poly ADP ribose polymerase (PARP) inhibitor, one or more ataxia telangiectasia and Rad3-related (ATR) signaling inhibitor, and combinations thereof. In certain embodiments, the one or more SIRT activator comprises resveratrol. In certain embodiments, the one or more HDAC inhibitor comprises SB-939, BRD-A19037878, or a combination thereof. In certain embodiments, the PARP inhibitor comprises veliparib. In certain embodiments, the ATR signaling inhibitor comprises VE-821.

In certain embodiments, the treatment timing and/or concentration of the one or more rejuvenating agent is varied. In certain embodiments, the one or more rejuvenating agent is administered at a concentration of between about 0.01 and 5 μM, or any value in between, for example, between about 0.02 and 5 μM, or between about 0.03 and 5 μM, or between about 0.4 and 5 μM, or between about 0.05 and 5 μM, or between about 0.1 and 5 μM, or between about 0.5 and 5 μM, or between about 1 and 5 μM, or between about 2 and 5 μM, or between about 3 and 5 μM, or between about 4 and 5 μM, or between about 0.01 and 4 μM, or between about 0.01 and 3 μM, or between about 0.01 and 2 μM, or between about 0.01 and 1 μM, or between about 0.01 and 0.5 μM, or between about 0.01 and 0.2 M, or between about 0.01 and 0.1 μM, or between about 0.01 and 0.05 μM. In certain embodiments, the one or more rejuvenating agent is administered at a concentration of between about 5 and 50 μM, or any values in between, for example between about 10 and 50 M, or between about 15 and 50 μM, or between about 20 and 50 μM, or between about 30 and 50 μM, or between about 40 and 50 μM, or between about 5 and 40 μM, or between about 5 and 30 μM, or between about 5 and 20 μM, or between about 5 and 15 μM, or between about 5 and 10 μM. In certain embodiments, the one or more rejuvenating agent is administered at a concentration of about 0.01 μM, about 0.02 μM, about 0.03 μM, about 0.04 μM, about 0.05 μM, about 0.06 μM, about 0.07 μM, about 0.08 μM, about 0.09 UM, about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, or about 50 μM.

In certain embodiments, the one or more rejuvenating agent described herein is contacted to a cell for at least about 1 day, at least about 2 days, at least about 3 days, at least about 4 days, at least about 5 days, at least about 6 days, at least about 7 days, at least about 8 days, at least about 9 days, at least about 10 days. In certain embodiments, the one or more rejuvenating agent is contacted to the cells for up to about 1 day, up to abut 2 days, up to about 3 days, up to about 4 days, up to about 5 days, up to about 6 days, up to about 7 days, up to about 8 days, up to about 9 days, up to about 10 days. In certain embodiments, the one or more rejuvenating agent is contacted to the cells for about 1 day, about 2 days, about 3 days, about 4 days, about 5 days, about 6 days, about 7 days, about 8 days, about 9 days, or about 10 days.

The present disclosure provides a method for rejuvenating cellular age in a cell or population of cells, comprising contacting the cell or population of cells with one or more rejuvenating agent. In certain embodiments, the cell or population of cells comprise fibroblasts. In certain embodiments, the one or more rejuvenating agent is selected from the group consisting of one or more histone deacetylase (HDAC) inhibitor, one or more heat shock protein (HSP) inhibitor, one or more adrenergic receptor antagonist, one or more phosphoinositide 3-kinase (PI3K) inhibitor, and combinations thereof. In certain embodiments, the one or more HDAC inhibitor comprises mocetinostat. In certain embodiments, the one or more HSP inhibitor comprises radicicol, geldanamycin, or a combination thereof. In certain embodiments, the one or more adrenergic receptor antagonist comprises alfuzosin. In certain embodiments, the one or more PI3K inhibitor comprises wortmannin. In certain embodiments, the cell or population of cells comprise neurons, motoneurons, cortical neurons, peripheral sensory neurons, or midbrain dopamine neurons. In certain embodiments, the one or more rejuvenating agent is selected from the group consisting of one or more Sirtuin (SIRT) activator, one or more HDAC inhibitor, one or more poly ADP ribose polymerase (PARP) inhibitor, one or more ataxia telangiectasia and Rad3-related (ATR) signaling inhibitor, and combinations thereof. In certain embodiments, the one or more SIRT activator comprises resveratrol. In certain embodiments, the one or more HDAC inhibitor comprises SB-939, BRD-A19037878, or a combination thereof. In certain embodiments, the PARP inhibitor comprises veliparib. In certain embodiments, the ATR signaling inhibitor comprises VE-821. In certain embodiments, contacting the cell or population of cells with one or more rejuvenating agent increases the expression level or presence in the cell or population of cells of one or more chronological marker signature of a young cell. In certain embodiments, contacting the cell or population of cells with one or more rejuvenating agent decreases the expression level or presence in the cell or population of cells of one or more chronological marker signature of an old cell. In certain embodiments, contacting the cell or population of cells with one or more rejuvenating agent reduces loss of heterochromatin, disrupted nuclear lamina, DNA damage, cellular senescence, disrupted proteostasis and dependence on autophagy, caveolin-1 (CAV1) expression, or a combination thereof. In certain embodiments, the cell or population of cells are derived from primary cells, fetal cells, pluripotent stem cells, embryonic stem cells, induced pluripotent stem cells, or combinations thereof. In certain embodiments, the cell or population of cells are human.

The present disclosure provides a rejuvenated cell, wherein the rejuvenated cell is produced by a method comprising contacting a cell with one or more rejuvenating agent. In certain embodiments, the rejuvenated cell is derived from fibroblasts. In certain embodiments, the one or more rejuvenating agent is selected from the group consisting of one or more histone deacetylase (HDAC) inhibitor, one or more heat shock protein (HSP) inhibitor, one or more adrenergic receptor antagonist, one or more phosphoinositide 3-kinase (PI3K) inhibitor, and combinations thereof. In certain embodiments, the one or more HDAC inhibitor comprises mocetinostat. In certain embodiments, the one or more HSP inhibitor comprises radicicol, geldanamycin, or a combination thereof. In certain embodiments, the one or more adrenergic receptor antagonist comprises alfuzosin. In certain embodiments, the one or more PI3K inhibitor comprises wortmannin. In certain embodiments, the rejuvenated cell is derived from neurons, motoneurons, cortical neurons, peripheral sensory neurons, or midbrain dopamine neurons. In certain embodiments, the one or more rejuvenating agent is selected from the group consisting of one or more Sirtuin (SIRT) activator, one or more HDAC inhibitor, one or more poly ADP ribose polymerase (PARP) inhibitor, one or more ataxia telangiectasia and Rad3-related (ATR) signaling inhibitor, and combinations thereof. In certain embodiments, the one or more SIRT activator comprises resveratrol. In certain embodiments, the one or more HDAC inhibitor comprises SB-939, BRD-A19037878, or a combination thereof. In certain embodiments, the PARP inhibitor comprises veliparib. In certain embodiments, the ATR signaling inhibitor comprises VE-821. In certain embodiments, the rejuvenated cell is derived from primary cells, fetal cells, pluripotent stem cells, embryonic stem cells, induced pluripotent stem cells, or combinations thereof. In certain embodiments, the rejuvenated cell is human. In certain embodiments, contacting the cell with one or more rejuvenating agent increases the expression level or presence of one or more chronological marker signature of a young cell. In certain embodiments, contacting the cell with one or more rejuvenating agent decreases the expression level or presence of one or more chronological marker signature of an old cell. In certain embodiments, contacting the cell with one or more rejuvenating agent reduces loss of heterochromatin, disrupted nuclear lamina, DNA damage, cellular senescence, disrupted proteostasis and dependence on autophagy, caveolin-1 (CAV1) expression, or a combination thereof.

### 5.3.3 Disease Modeling

The present disclosure also relates, in certain embodiments, to cells, methods, and systems for modeling age-related neurodegenerative diseases (e.g., AD) in vitro. Such disease models can be developed by inducing aging chronologic marker signatures in somatic cells (not necessarily derived by induced differentiation of stem cells) that express a “young” marker signature. This strategy can be applied to cell cultures derived from a patient with a late-onset disease and/or disorder including, but not limited to, a neurodegenerative disease, such as Alzheimer's disease (AD) or Parkinson's disease (PD), to derive age-appropriate cell cultures that more accurately represent patient age and thus the disease state.

Induced cellular aging provides a system to model age-related aspects of late-onset neurodegenerative diseases. Such a system can be used to directly test an interaction between genetic susceptibility and age-related vulnerability on disease phenotype.

Disease phenotypes may, in some instances, be based upon aging and/or genetic susceptibility. Accordingly, the present disclosure provides methods for inducing aging to examine late-onset disease and/or disorders in age-appropriate iPSC-based cell culture models, which are characterized by the induction and display of one or more chronological marker signatures, and optionally one or more disease signatures (including for example genetic pre-disposition). In certain embodiments, contacting the cell with one or more age-inducing agent induces loss of heterochromatin, disrupted nuclear lamina, increased DNA damage, increased cellular senescence, disrupted proteostasis and increased dependence on autophagy, increased caveolin-1 (CAV1) expression, or a combination thereof.

The present disclosure provides a method of modeling neurodegenerative disease, comprising: contacting a cell or population of cells with one or more age-inducing agent to obtain an age-modulated cell or population of age-modulated cells; and contacting the age-modulated cell or population of age-modulated cells with one or more candidate compound. In certain embodiments, the method further comprises detecting an alteration in at least one of the survival, biological activity, structure or morphology of the age-modulated cell or population of age-modulated cells. In certain embodiments, the method further comprises selecting as the drug a candidate one or more compound that alters at least one of the survival, biological activity, structure or morphology of the age-modulated cell or population of age-modulated cells. In certain embodiments, the neurodegenerative disease is Parkinson's disease (PD) or Alzheimer's Disease (AD).

### 5.4 Methods of Treatment

Cells may be isolated from healthy subjects, at risk subjects, and diseased subjects for use in generating undifferentiated iPS cells according to methodology presented herein or as otherwise available in the art. Primary somatic cells used for reprogramming may be isolated from a variety of bodily locations, such as circulating cells and/or cells in tissues of patients/subjects, including but not limited to fibroblasts, skin fibroblasts, white blood cells, circulating white blood cells, mucosal cells, and keratinocytes without regard for the “age” of the cell or the “age” of the donor. In some aspects, primary somatic cells may be young cells expressing a “young” cell marker signature isolated from young donors, which cells may or may not be expressing a disease signature. In other aspects, primary somatic cells may be old cells expressing an “old” marker signature. In further aspects, primary somatic cells may be cells expressing a disease marker signature regardless of the chronological age of the donor. These primary cells can be reprogrammed in culture to give rise to iPSC using any method for generating iPSC from somatic cells. Such methods, other than described or referenced herein, are known in the art.

Generated iPSCs of any origin, including cells generated by methods described herein, may be used in differentiation protocols for producing differentiating and differentiated iPSC-derived cells that may find use in hypomethylation aging compositions and methods of the present disclosures. Differentiating and differentiated iPSC-derived cells include but are not limited to default and nondefault differentiation lineages, including partially differentiated (i.e., differentiating) cells, so long as they are capable of expressing genetic and cell marker signatures of their particular cell types, i.e., permissive cells. Examples of cell types which may find use in aging induction using methods of the present disclosure are iPSC-derived cells including but not limited to neurons (any subtype, such as motoneurons, cortical neurons, peripheral sensory neurons, mid-brain dopamine neurons etc.), and fibroblasts.

Thus, iPSC derived cells at certain stages will find use in induced aging or rejuvenation treatments according to the present disclosure including, but not limited to, iPSC derived cells beginning to undergo differentiation, iPSC derived cells progressing towards committed cells types, iPSC derived cells progressing towards a mature cell type, etc. For example, an iPSC-derived midbrain dopamine neuron, or precursor thereof, (for example, from a healthy subject) can be aged according to the present disclosure and can be used in a cell based therapy for introducing into a PD patient. Accordingly, the present disclosure provides for pharmaceutical compositions comprising the aged cells described herein.

Non-limiting examples of specific iPSC-derived cell types and associated disease(s) which can be used in conjunction with the methods of inducing aging described herein include iPSC derived-neurons for neurodegenerative diseases, iPSC-derived motoneurons for ALS, iPSC-derived cortical neurons for Alzheimer's, iPSC-derived mDA neurons and iPSC-derived cortical neurons for corticobasal degeneration, iPSC-derived astrocytes for neurodegenerative disorders, and the like.

The presently disclosed cells and the pharmaceutical compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently cells.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed cells. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the presently disclosed cells is the quantity of cells necessary to achieve an optimal effect. An optimal effect include, but are not limited to, repopulation of the CNS of a subject suffering from a neurological disorder (e.g., PD or AD), and/or improved function of the subject's CNS.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the neurological disorder (e.g., PD or AD), or otherwise reduce the pathological consequences of the neurological disorder (e.g., PD or AD). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the presently disclosed cells is an amount that is sufficient to repopulate the CNS of a subject suffering from a neurological disorder (e.g., PD or AD). In certain embodiments, an effective amount of the presently disclosed cells is an amount that is sufficient to improve the function of the CNS of a subject suffering from a neurological disorder (e.g., PD or AD), e.g., the improved function can be about 1%, about 5%, about 10%, about 20%, about 30%, about 40%, about 50%, about 60%, about 70%, about 80%, about 90%, about 95%, about 98%, about 99% or about 100% of the function of a normal person's CNS.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed cells are administered to a subject. In certain embodiments, from about 1×10⁵ to about 1×10⁷ of the presently disclosed s cells are administered to a subject suffering from a neurological disorder (e.g., PD or AD). In certain embodiments, about 2×10⁵ of the presently disclosed cells are administered to a subject suffering from a neurological disorder (e.g., PD or AD). In certain embodiments, from about 1×10⁶ to about 1×10⁷ the presently disclosed cells are administered to a subject suffering from a neurological disorder (e.g., PD or AD). In certain embodiments, from about 2×10⁶ to about 4×10⁶ the presently disclosed cells are administered to a subject suffering from a neurological disorder (e.g., PD or AD). The precise determination of what would be considered an effective dose may be based on factors individual to each subject, including their size, age, sex, weight, and condition of the particular subject. Dosages can be readily ascertained by those skilled in the art from this disclosure and the knowledge in the art.

In certain embodiments, the cells that are administered to a subject suffering from a neurological disorder (e.g., PD or AD) for treating a neurological disorder are a population of midbrain dopamine neurons that are differentiated and aged according to the methods described herein. In certain embodiments, the cells that are administered to a subject suffering from a neurological disorder (e.g, PD or AD) for treating a neurological disorder are a population of midbrain dopamine neuron precursors that are differentiated and aged according to the methods described herein.

The present disclosure provides a composition comprising the age-induced cell disclosed herein or the rejuvenated cell disclosed herein. In certain embodiments, the composition is a pharmaceutical composition. In certain embodiments, the composition further comprises a pharmaceutically acceptable carrier.

### 5.5 Methods of Screening Therapeutic Compounds

In certain embodiments, aged iPSC-derived cell types obtained as described herein may find use in disease modeling and for identifying therapeutically relevant cell stages during development, such as identifying aged cellular stages for use in testing new drug compounds for use as therapeutics and for actual use in treatment of patients. Thus in some embodiments, primary somatic cell donors for iPSC-derived cell types have a disease or a disease phenotype induced in iPSC-derived cell/tissue culture including but are not limited to actual or model neurodegenerative diseases, such as Parkinson's disease (PD), Alzheimer's disease (AD), tauopathies, i.e., a class of neurodegenerative diseases associated with the pathological aggregation of tau protein in the human brain, etc.

The presently disclosed aged cells can be used to model disorders, for example, a neurological disorder such as Parkinson's disease (PD) and Alzheimer's disease (AD), and serve as a platform to screen for candidate compounds that can overcome disease related defects. The capacity of a candidate compound to alleviate a disorder (e.g., PD or AD) can be determined by assaying the candidate compound's ability to rescue a physiological or cellular defect in a diseased cell, for example, an iPSC-derived midbrain dopamine neuron (mDA), or precursor thereof, wherein the iPSC is prepared from a somatic cell obtained from a PD patient.

The presently disclosed subject matter provides for methods of screening compounds suitable for treating a disorder (e.g., PD or AD) in vitro. In certain embodiments, the method comprises identifying a compound that is capable of rescuing at least one cellular disease phenotype.

In certain embodiments, the method comprises: (a) providing (i) a population of the presently disclosed aged cells (e.g., iPSC-derived PD neurons or progenitors thereof), and (ii) a test compound; (b) contacting the cells with the test compound; and (c) measuring the level or presence of one or more disease phenotype, for example, wherein a test compound that reduces the level of presence of the one or more disease phenotype is selected as a candidate therapeutic compound.

### 5.6 Kits

The presently disclosed subject matter provides for kits for inducing aging and/or maturation of a cell, for example, an iPSC-derived cell, such as an iPSC-derived somatic cell (e.g., iPSC-derived fibroblasts and iPSC-derived neurons), wherein the expression of one or more chronological markers of an aged cell is increased following contact of the cell with one or more age-inducing agent. In certain embodiments, the kit comprises one or more age-inducing agent that induces or increases expression of one or more chronological markers of an aged cell. In certain embodiments, the kit comprises instructions for inducing age in the cell, such that the expression of one or more chronological markers of an aged cell is increased in the cell following treatment of the cell according to the instructions. In certain embodiments, one or more chronological markers of a young cell is decreased in the cell following treatment of the cell according to the instructions.

The presently disclosed subject matter provides for kits for reducing aging and/or maturation of a cell, for example, an iPSC-derived cell, such as an iPSC-derived somatic cell (e.g., iPSC-derived fibroblasts and iPSC-derived neurons), wherein the expression of one or more chronological markers of a young cell is increased following contact of the cell with one or more rejuvenating agent. In certain embodiments, the kit comprises one or more rejuvenating agent that induces or increases expression of one or more chronological markers of a young cell. In certain embodiments, the kit comprises instructions for reducing age in the cell, such that the expression of one or more chronological markers of a young cell is increased in the cell following treatment of the cell according to the instructions. In certain embodiments, one or more chronological markers of an aged cell is decreased in the cell following treatment of the cell according to the instructions.

In certain embodiments, the kit comprises instructions for administering a population of the presently disclosed cells, for example, stem-cell-derived neurons, such as midbrain dopamine neurons, or precursors thereof, or a composition comprising said cells, to a subject suffering from a disorder, such as a neurological disorder, for example, Parkinson's disease or Alzheimer's disease. The instructions can comprise information about the use of the cells or composition for treating or preventing the disorder. In certain embodiments, the instructions comprise at least one of the following: description of the therapeutic agent; dosage schedule and administration for treating or preventing the disorder, or symptoms thereof; precautions; warnings; indications; counter-indications; over dosage information; adverse reactions; animal pharmacology; clinical studies; and/or references. The instructions can be printed directly on a container (when present) comprising the cells, or as a label applied to the container, or as a separate sheet, pamphlet, card, or folder supplied in or with the container.

The present disclosure further provides a kit for rejuvenating a cell or population of cells, comprising one or more rejuvenating agent selected from the group consisting of one or more histone deacetylase (HDAC) inhibitor, one or more heat shock protein (HSP) inhibitor, one or more adrenergic receptor antagonist, one or more phosphoinositide 3-kinase (PI3K) inhibitor, one or more Sirtuin (SIRT) activator, one or more HDAC inhibitor, one or more poly ADP ribose polymerase (PARP) inhibitor, one or more ataxia telangiectasia and Rad3-related (ATR) signaling inhibitor, and combinations thereof. In certain embodiments, the one or more HDAC inhibitor comprises mocetinostat. In certain embodiments, the one or more HSP inhibitor comprises radicicol, geldanamycin, or a combination thereof. In certain embodiments, the one or more adrenergic receptor antagonist comprises alfuzosin. In certain embodiments, the one or more PI3K inhibitor comprises wortmannin. In certain embodiments, the one or more SIRT activator comprises resveratrol. In certain embodiments, the one or more HDAC inhibitor comprises SB-939, BRD-A19037878, or a combination thereof. In certain embodiments, the PARP inhibitor comprises veliparib. In certain embodiments, the ATR signaling inhibitor comprises VE-821. In certain embodiments, the kit further comprises instructions for contacting a cell or population of cells with the one or more rejuvenating agent to induce expression in the cell or population of cells of one or more chronological marker signature of a young cell.

### 6. EXAMPLES

The presently disclosed subject matter will be better understood by reference to the following Example, which is provided as exemplary of the presently disclosed subject matter, and not by way of limitation.

### Example 1

### Identifying Age-Modulating Agents and Quantifying Cellular Aging Using a Novel Computational Framework for Evaluating Transcriptional Age

The differentiation of human pluripotent stem cells (hPSCs) provides access to a wide range of cell types and tissues. However, hPSC-derived lineages typically represent a fetal stage of development, and methods to expedite the transition to an aged identity are limited. RNAge, a transcriptome-based computational platform, enables the evaluation of induced aging or rejuvenated states. This approach is validated across independent datasets spanning different tissues and species. RNAge can be used to evaluate the effectiveness of existing age modulating interventions. RNAge is used to perform an in silico compound screen using the LINCS L1000 dataset. This approach led to the identification and experimental confirmation of several novel compounds capable of inducing aging or rejuvenation in primary fibroblasts or hPSC-derived neurons. In addition, applying such compounds to hPSC-models of Alzheimer's disease neurodegeneration in a genotype specific manner. RNAge offers a robust method for quantifying age-related manipulations and unveils compounds that significantly broaden the toolkit for age-modifying strategies in hPSC-derived lineages.

### Methods

Reprogramming of primary fibroblasts to iPSCs. Young and old primary fibroblasts were obtained from Coriell and maintained in α-MEM+15% FBS. Fibroblasts were reprogrammed to iPSCs using CytoTune Sendai viruses expressing SOX2, OCT4, KLF4 and c-MYC. After the formation of iPSC colonies (aprox. 30 days after transduction), individual colonies were manually isolated, replated onto MEFs and maintained in KSR media.

Differentiation of iPSCs to fibroblasts. Differentiation of iPSCs into fibroblasts was performed. In brief, pluripotent stem cells were cultured on MEFs in KSR based media supplemented with 10 ng/ml FGF2. For differentiation, colonies were dissociated from the feeder layer using dispase and replated onto gelatin coated plates. Cells were maintained in DMEM+20% heat-inactivated fibroblasts for 25 days with passaging ever 5-6 days. Fibroblasts were isolated by flow cytometry (CD-13^{high} and HLA-CD44^{high}) after 25 days of culture and cultured for an additional 7 days before harvest.

Fibroblast maintenance and culture. Fibroblasts were maintained in DMEM-F12 containing GlutaMAX,15% heat inactivated FBS and 1× Pen/Strep and passaged using Trypsin. For validation of L1000 aging score by RNA-seq old (GM04204; Coriell) or young (HDF-f; ScienCell #2300) fibroblasts were plated at a density of 4000 cm², test compounds were applied when cultures reached 70% confluence.

Generation of human cortical neurons. Cortical neurons were generated by directed differentiation of human embryonic stem cells (H9; WA-09) maintained in E8 on vitronectin coated plates. Differentiation was performed by seeding pluripotent stem cells onto Matrigel coated dishes at a density of 300,000/cm² in the presence of Y27632 (10 μM). After 24 h the E8 medium was replaced with E6 containing SB431542 (10 μM), LDN193189 (100 nM) and XAV939 (2 M). After three days the XAV939 was removed from the differentiation media and cells were cultured for an additional seven days in E6 containing SB431542 (10 μM), LDN193189 (100 nM). The neuroepithelium was maintained for an additional 10 days in neurobasal supplemented with N2 and B27. Media was changed daily throughout the differentiation. At DIV 20 the cultures were dissociated using Accutase to generate a single cell suspension and plated out for subsequent experiments in Neurobasal medium supplemented with B27, L-glutamine, BDNF, CAMP, ascorbic acid and GDNF (neural maintenance media). DAPT was also added to the culture medium from DIV20 to DIV30.

Compounds for experimental validation of L1000-based score. All validation experiments were performed after 24 h of compound treatment. For rejuvenation experiments in fibroblasts Mocetinostat (10 μM; Selleck S1396), Resveratrol (0.37 μM; Selleck S1396) and Radicicol (1.1 μM; Tocris 1589), DMSO (1:1000) and EtOH (1:1000; vehicle control for Radicicol) were applied to old primary fibroblasts (Coriell GM4204). Age induction experiments in young fibroblasts (HDF-f; ScienCell #2300) were performed with 5-Iodotubercin (10 μM; Selleck S8314), Alvocidib (10 μM; Selleck S1230) and Mitoxantrone (1.1 μM; Selleck S2485) with DMSO (1:1000) used as a control. Compounds used for age induction experiments in PSC-derived neurons were: Fludarabine (10 μM; Selleck S1491) Vinorelbine (10 μM; sc-205885) and AGK-2 (0.04 μM; Selleck S7577) with DMSO (1:1000) used as a control.

Assaying Hallmarks of Age by high content microscopy. HDF fibroblasts were plated at a density of 4500/cm² and cultured for 48 h before drug treatment. Neurons were plated at a density of 100,000/cm² and cultured for 12 days before the application of candidate age inducers. After 24 h of treatment culture wells were washed 2× in PBS then fixed with 4% paraformaldehyde. For immunocytochemistry cells were permeabilized in PBS+0.3% Triton and blocked in 5% normal goat serum. All antibody primary incubations were performed overnight at 4 degrees and secondary antibodies at RT for 1h. Primary antibodies used in this study were: BAG3 (abcam; ab47124), CAV1 (Thermo; MA3-600), H3K9me3 (abcam; ab176916), LAP2 (BD Bioscience; 611000), p21 (CST; 2947), yH2AX (EMD Millipore; 05-636). Immunocytochemistry images were acquired using an Operetta (PerkinElmer, Waltham, MA) microscope and quantified using the harmony high content analysis software. For both fibroblasts and neurons 4 independent batches of cells were assayed in triplicate (n=12). Fluorescence intensity was normalized to DMSO, and ordinary one-way ANOVA used to compare test conditions to the DMSO control (Prism 9.5.1).

Viability Assay. To measure neuronal viability PrestoBlue Cell viability Reagent was diluted 1:10. A total of 85 ul of diluted reagent was applied to each 96 well, incubated for 2 h at 37 degrees then assayed. Each well was normalized to the mean absorbance of the DMSO control for each differentiation and technical replicates were averaged to give a single value for each differentiation.

RNA Extraction. Cell were harvested, lysed, and stored in Trizol (Life Technologies) until further processing. For primary frontal cortex or substantia nigra a total of 10 mg of brain tissue from was lysed in Trizol aided by a tissue dounce. For experimental validation L1000 compounds RNA was extracted by the core using miRneasy Micro Kit (QIAGEN 1071023). In all other cases RNA was extracted using the Zymo Direct-zol RNA Microprep Kit according to the manufacturer's instructions.

RNA-seq analysis. Reads were aligned to the hg19 Human transcripts using STAR (version 2.5.21b) using default parameters and resulting bam files were sorted and indexed using samtools. Gene counts were obtained using featureCounts (version 1.4.3) from sorted bam files using uniquely mapped reads. Genes with no expression counts in any sample were discarded. Differential gene expression analysis was performed using DESeq2 R package that normalize gene count data to transcription per million (TPM), and then detect differentially expressed genes (DEG) between Young and Old groups with (FDR<0.1).

RNAge Training datasets. Total pF: Primary fibroblast samples from 9 young (7-14 years old) and 9 old (70-96 years old) profiled by total-RNA-seq protocol. PolyA pF: Randomly picked 4 young (10-11 years old) and 4 old (71-96 years old) out of above 18 primary fibroblast samples. These samples were profiled by polyA RNA-seq kit. PolyA Gage pF: Primary fibroblast RNA-seq data from 6 young (<15-year-old) and 4 old (>70-year-old) were downloaded from E-MTAB-3037 dataset. All raw data were processed by our RNA-seq analysis protocol. PolyA Gage FC: frontal cortex RNA-seq data from 4 young (<15-year-old) and 4 old (>70-year-old) were downloaded from E-MTAB-3037 dataset. All raw data were processed by our RNA-seq analysis protocol. Total FC: Primary frontal cortex samples from 9 young (13-14 years old) and 9 old (70-91 years old) profiled by total-RNA-seq protocol. Total SN: Primary substantia nigra samples from 10 young (13-14 years old) and 9 old (70-91 years old) profiled by total-RNA-seq protocol.

Other datasets. GSE113957: Fleischer et al. collected fibroblast samples from 133 healthy individuals. The FPKM of gene expression were downloaded from GEO database. Out of 133 samples, 10 young (8-13 years old), 10 old (>89-year-old) and 10 HGPS (2-8 years old) samples were used in this study. GSE36192: Microarray data of the cerebellum and frontal cortex from 396 subjects (total 911 tissue samples) were generated by North American Brain Expression Consortium. The normalized gene expression matrix was downloaded from GEO database. Both cerebellum and frontal cortex from the same 18 young (10-15 years old) and 16 old (>92-year-old) samples were used in this study. GSE52431: The raw RNA-seq data from 4 iPSC-derived dopamine neurons with overexpressing progerin and 4 corresponding control samples were downloaded and processed with proposed pipeline. GSE132040: Bulk RNA-seq data from Tabula Muris Consortium study were downloaded and processed. The whole brain data from young (<3-month) and old (>24-month) mice were used in this study. GSE141028: Fathi et al. presented a study of inducing senescence by treating human embryonic stem cells with compounds. The raw RNA-seq data from 3 treatment and 3 WT cell lines were downloaded, processed, and used in this study. E-MTAB-5965: Genetic ablation of SATB1 induces a senescence phenotype in human embryonic stem cell (hESC)-derived DA neurons. The raw RNA-seq data from 3 STAB1-KO and 3 WT cell lines were downloaded, processed, and used in this study. E-MTAB-10352: The raw RNA-seq data from 42 samples were downloaded, processed, and used in this study, including 17 samples from iPSC derived induced neurons and 25 samples from direct conversion of human fibroblasts into induced neurons. GTEx: The FPKM data of four tissues was downloaded from GTEx Portal, including frontal cortex, cortex, substantia nigra and stomach.

Aging markers identification. We derived the aging signature for the different tissue types using the following six datasets:

$$D = \{ Total\_pF , PolyA\_pF , PolyA\_Gage \_pF , Total\_FC , PolyA\_Gage \_FC , Total\_SN \} .$$

Differential gene expression analysis was performed using DESeq2 between young and old samples in each of the RNA-seq datasets, resulting in log fold change values and p-value from each of the six datasets. The log 2 based fold change between young and old groups for each gene i in each dataset j is denoted as L_{i,j}, where i∈G, j∈D. The p-value between young and old groups for each gene in each dataset is denoted as P_{i,j}, where i∈G, j∈D. We discretized the log 2 fold change of the genes to indicate the regulatory direction in aging. We defined a simplified log 2 fold change as follow:

$${\underline{L}}_{i , j} = \{ \begin{matrix}0 & 1 & - 1 & \frac{if - 0.1 < {L}_{i , j} < 0.1}{if {L}_{i , j} \geq 0.1 if {L}_{\{ i , j \}} \leq - 0.1} & i ∈ G , j ∈ D\end{matrix}$$

Then we combined p-values from all six dataset for each gene with Edgington's method:

$${C}_{i} = Edgingto n ( {P}_{i , j} ) i ∈ G , j ∈ D$$

Based on the simplified L_{i,j}, we assign genes to discrete and mutually exclusive aging signatures in hierarchical scheme as follow:

$$\begin{matrix}1. {G}_{1} = \{ Universal aging gene i \} & if abs \begin{matrix}( {\sum }_{j ∈ D} & {\underline{L}}_{i , j} ) = 6 ,\end{matrix} i ∈ G \\ 2. {G}_{2} = \{ Fibroblast aging gene i \} & if abs \begin{matrix}( {\sum }_{j} & {\underline{L}}_{i , j} ) = 3\end{matrix} \\ where i ∈ ( G ⋂ \underline{{G}_{1}} ) , j ∈ \{ Total\_pF , PolyA\_pF , PolyA\_Gage \_pF \} & \\ 3. {G}_{3} = \{ Pan neuronal aging gene i \} & if abs \begin{matrix}( {\sum }_{j} & {\underline{L}}_{i , j} ) = 3\end{matrix} \\ where i ∈ ( G ⋂ \underline{{G}_{1}} ⋂ \underline{{G}_{2}} ) , j ∈ \{ Total\_FC , PolyA\_Gage \_FC , Total\_SN \} & \\ 4. {G}_{4} = \{ FCtx aging gene i \} & if abs \begin{matrix}( {\sum }_{j} & {\underline{L}}_{i , j} ) = 2\end{matrix} \\ where i ∈ ( G ⋂ \underline{{G}_{1}} ⋂ \underline{{G}_{2}} ⋂ \underline{{G}_{3}} ) , j ∈ \{ Total\_FC , PolyA\_Gage \_FC \} & \\ 5. {G}_{5} = \{ SN aging genes i \} & if {\underline{L}}_{i , j} \neq 0 \\ where i ∈ ( G ⋂ \underline{{G}_{1}} ⋂ \underline{{G}_{2}} ⋂ \underline{{G}_{3}} ⋂ \underline{{G}_{4}} ) , j ∈ \{ Total\_SN \} & \end{matrix}$$

For each aging signature set, we ranked genes based on their combined p-value Cᵢ. Thus top 100 genes with smallest p-values from each subset were selected as the markers for the downstream analysis. These top ranked gene subsets denoted as Ĝ₁, Ĝ₂, Ĝ₃, Ĝ₄, Ĝ₅, respectively. According to the definition, the simplified L_{i,j} of above signatures is consistent across multiple datasets from the corresponding tissue type and it excludes genes that do not exhibit any change between the young and old groups. We can further define a marker vector for a tissue type k as

$${M}_{k} = \{ {\underline{L}}_{i , j} \vert i ∈ {\hat{G}}_{k} , j ∈ any datasets corresponding to {\hat{G}}_{k} \} = \{ {\underline{L}}_{i} \vert i ∈ {\hat{G}}_{k} \}$$

RNAge score calculation. The aging score is applied on a gene expression test data where two conditions are compared for expression changes in the aging signature, group1 has n samples and group2 has m samples. For each gene in a tissue specific marker set Ĝₖ, we can calculate t-statistic between two groups based on Welch's two-sample t test as follow:

$${t}_{i} = \frac{{μ}_{i , group 1} - {μ}_{i , group 2}}{\sqrt{\frac{{s}_{i , group 1}^{2}}{n} + \frac{{s}_{i , g r oup 2}^{2}}{m}}} i ∈ {\hat{G}}_{k}$$

where μ_{i,group1} is the mean expression of gene i in group1, μ_{i,group2} is the mean expression of gene i in group2,

$${s}_{i , group 1}^{2}$$

is the variance of gene i in group1, and

$${s}_{i , group 2}^{2}$$

is the variance of gene i in group2.

All t-statistics from the same tissue specific marker set Ĝₖ denoted as a vector Tₖ={i∈Ĝₖ}. In cases where marker gene i is not expressed in the test dataset we set tᵢ=0. Then we defined the “Aging score” for given tissue type k as follow:

$$Aging {score}_{k} = \frac{〈 {T}_{k} , {M}_{k} 〉}{{N}_{k}} = \frac{\begin{matrix}{\sum }_{i ∈ {\hat{G}}_{k}} & ( {t}_{i} \times {\underline{L}}_{i} )\end{matrix}}{{N}_{k}}$$

where the normalizing factor Nₖ is the number of overlapping genes between the test dataset and Ĝₖ. The aging score, Aging scoreₖ, represents the overall magnitude of expression difference between two sample groups based on the expression trends. This score is calculated using the tissue-specific aging marker set Mₖ. It indicates the aging difference between the two groups as well as distinguishes which group is older. To mitigate the influence of a small number of markers with large changes gene expression between group1 and group2 of the test data (i.e. large t-score values), we introduced the “Percentage score” as a measure of agreement between the expression trends of the aging markers and the given dataset. The calculation of the Percentage score involves the product of each gene between Tₖ and Mₖ: Aᵢ=tᵢ×Lᵢ, where i∈Ĝₖ. A positive Aᵢ indicates a consistent expression change of gene i in both the aging signature and in the comparison between group1 and group2 in the test dataset. Conversely, a negative Aᵢ indicates an opposite expression trend. Therefore, the “Percentage score” is defined as:

$$Percentage {score}_{k} = \frac{number of positive {A}_{i}}{{N}_{k}} , i ∈ {\hat{G}}_{k}$$

### Results

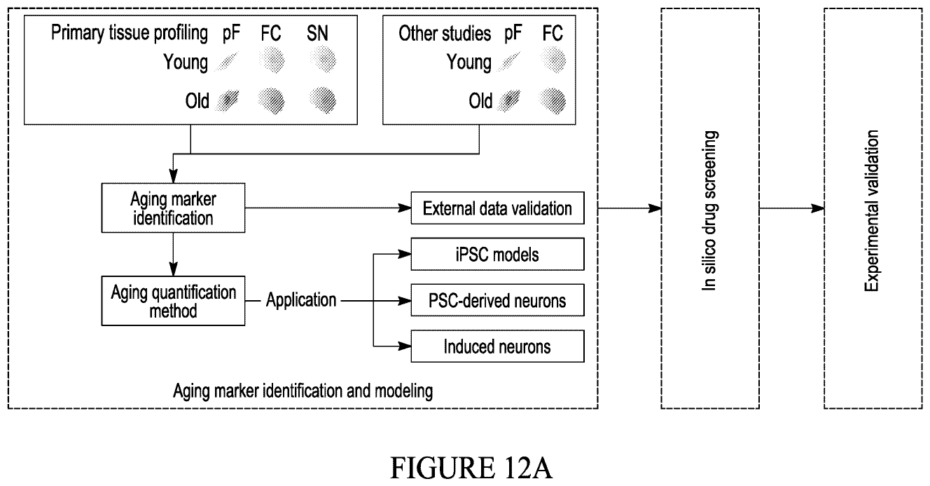

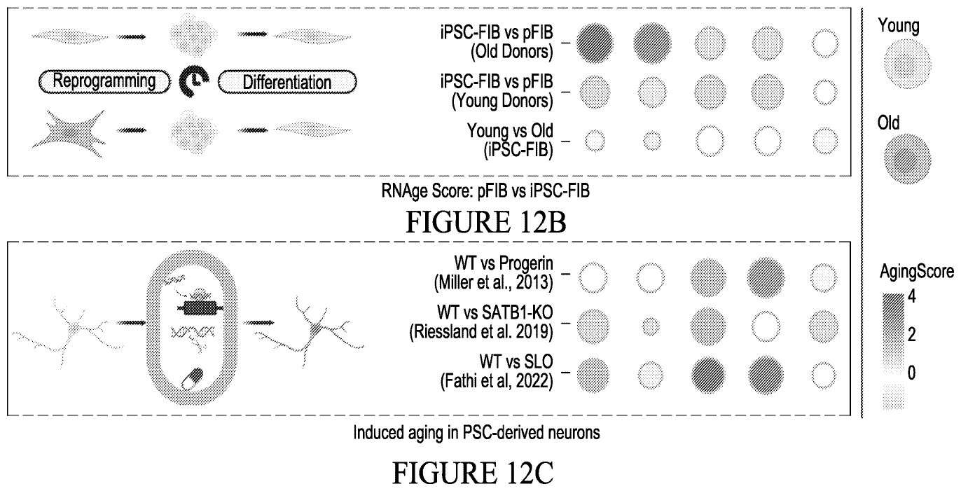

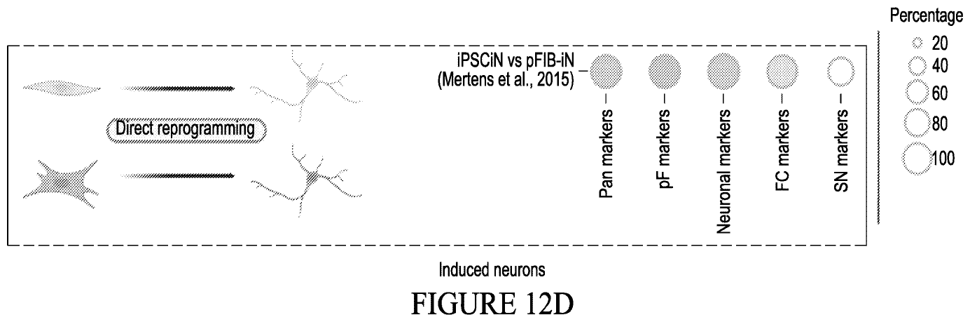

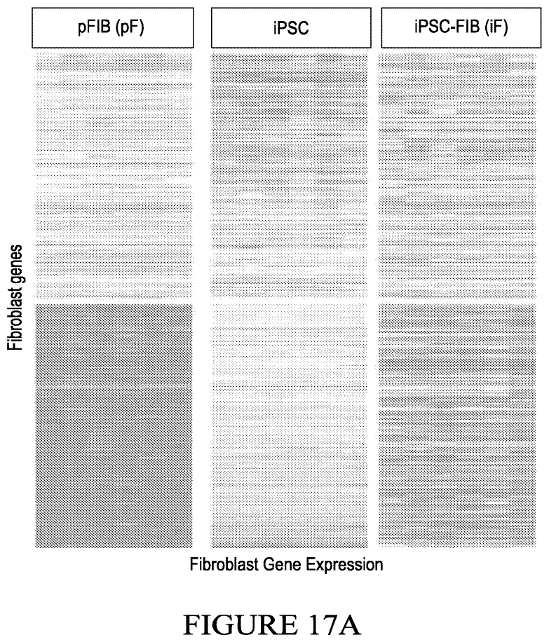

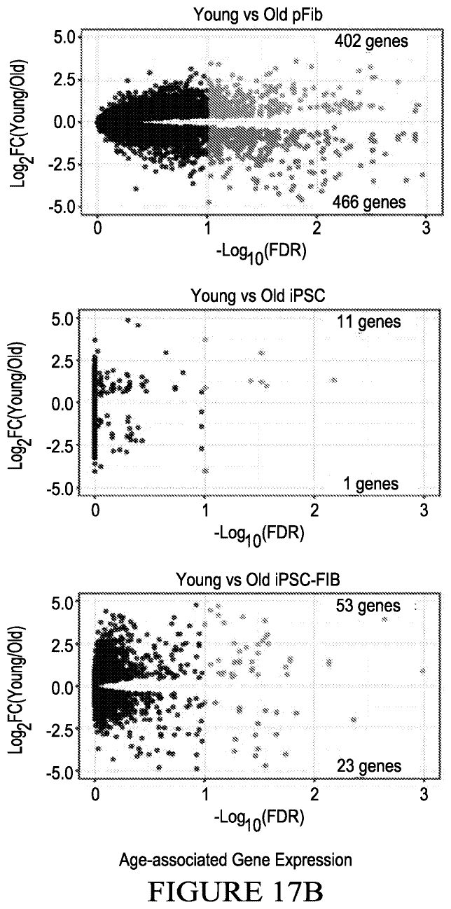

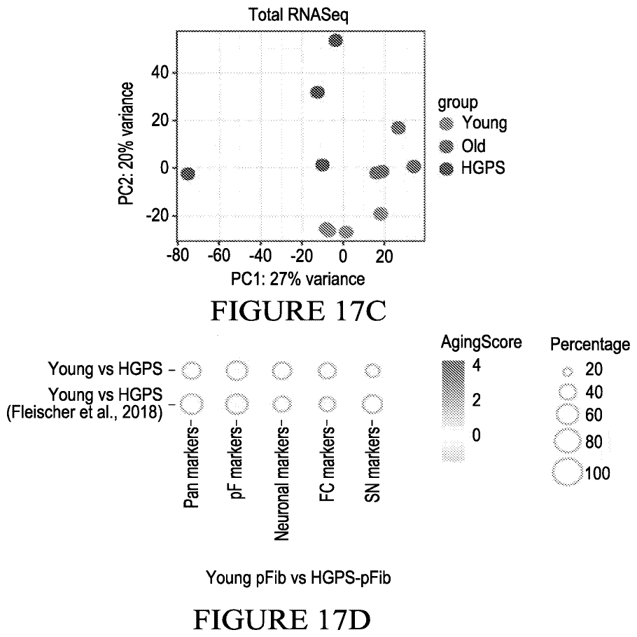

Measuring cellular rejuvenation in stem cell models. Transcriptional reprogramming of primary fibroblasts to pluripotent stem cells is one of the best described methods of resetting cellular age. As a positive control for RNAge, a subset of fibroblast lines were reprogrammed to pluripotent stem cells then differentiated them back into iPSC-derived fibroblasts (FIG. 12B). After confirming that reprogramming and re-differentiation was successful (FIG. 17A), RNA-seq was performed, and RNAge was applied. Reprogramming fibroblasts to iPSCs resulted in the loss of age-related differential expressed genes (DEGs) that were not re-established once iPSCs were differentiated back into iPSC-derived fibroblasts (FIG. 17B). This matched the RNAge score where young and old primary fibroblasts show an increased aging score relative to iPSC derived fibroblasts. Conversely, there was no difference in the aging score between iPSC-derived fibroblasts from young and old donors despite the large difference in age of the original donors. Together, these results further validate the RNAge score, support previous findings that aging signatures are erased during reprogramming and validates the use of the aging score in measuring cellular rejuvenation.

Evaluating manipulations that induce cellular age. Age induction strategies are critical for the study of late onset diseases in hPSC-based models and open new avenues for identifying and validating novel therapeutic interventions. Therefore, it was critical to determine whether RNAge can be used to compare cellular age in existing hPSC-based induced aging models (FIG. 12C). To date, at least three distinct age induction strategies have been described in neurons including: the ectopic expression of Progerin (responsible for HGPS), the knockout of SATB1 in dopaminergic neurons resulting in increased cellular senescence and the chemical induction of senescence in fibroblast and neurons using the “SLO” small molecule cocktail. Using the novel aging scoring methods, the ectopic expression of Progerin in iPSC-derived neurons triggered a pan ‘Neuronal’ and ‘FC’ aging signature but not an increase in the ‘Pan’ aging score. This is consistent with results from primary HGPS fibroblasts that did not show a ‘Pan’ aging, or ‘pF’ aging score (FIG. 17C-17D). Previous studies have also shown that SATB1 and the small molecule cocktail SLO can induce cellular aging hallmarks. Consistent with this, SATB1 KO and SLO treatment was observed to induce a robust increase in the transcriptional aging score in the expected subscores (FIG. 12C).

Comparison of induced aging approaches to direct reprogramming strategies. An alternative approach to incorporate age into stem cell models is to directly convert primary fibroblasts from young and old individuals to induced neurons (iNs) by the forced expression of Ngn2 and Ascl1. iNs are derived from fibroblasts of old individuals retain a transcriptional aging signature and show age-associated disease phenotypes in models of neurodegeneration. iNs generated from old fibroblasts were tested to determine whether they had a higher aging score compared to iNs generated from PSCs where cellular age is reset during reprogramming (FIG. 12D). The aging score showed that forebrain-like neurons generated from old fibroblasts displayed an increase in the ‘Pan’, ‘Neuronal’, and ‘FC’-specific aging scores but not the ‘SN’ aging score. However, unexpectedly, these fibroblast-derived neurons also showed expression of a fibroblast-specific ‘pF’ aging score, suggesting that they maintained age-related features of the original fibroblast, an aging signature that is not present in neurons that have undergone chronological aging (compare to FIG. 16F).

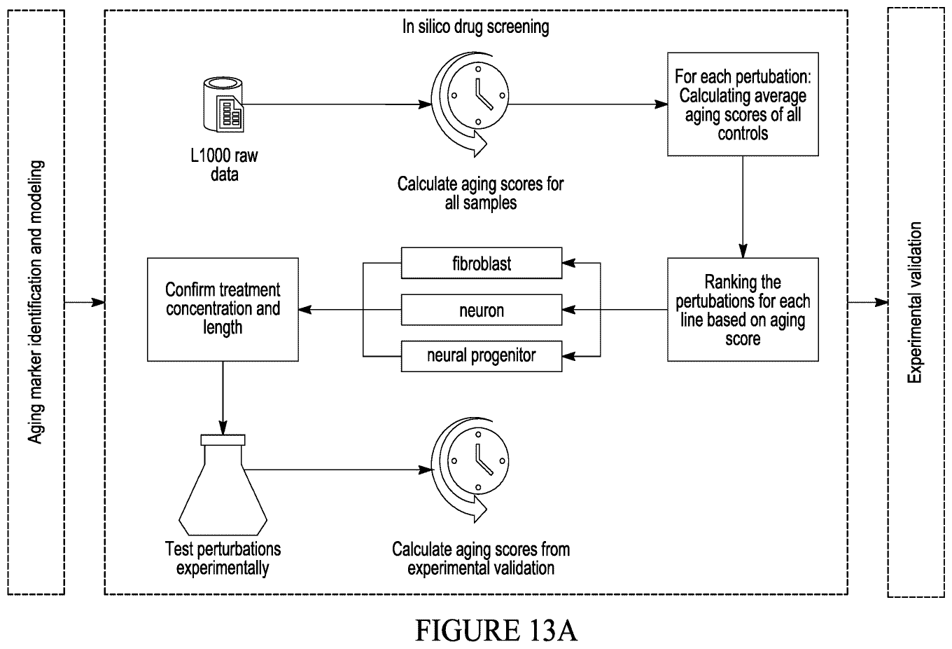

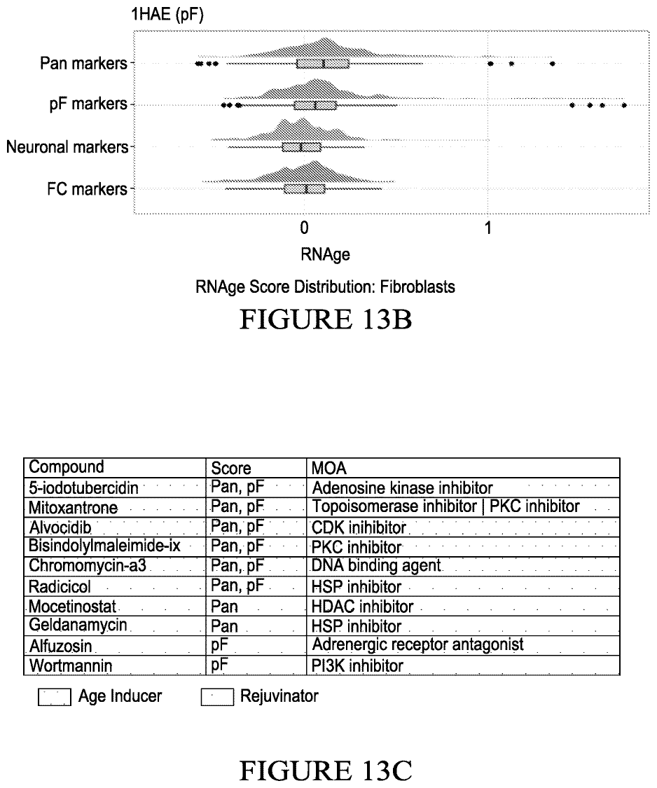

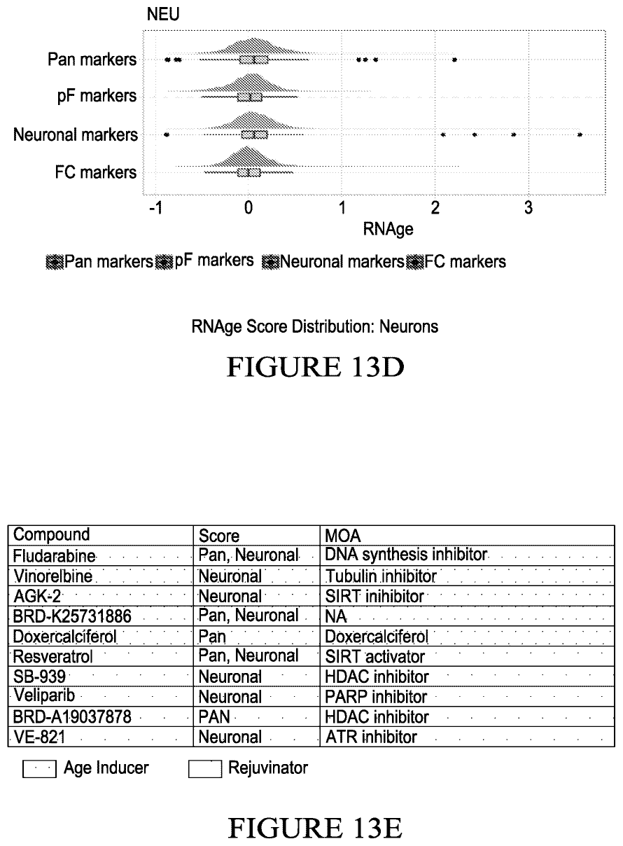

RNAge score is tractable and can be used to identify age-regulating compounds. RNAge can be used to compare the relative age of primary tissues and hPSC-derived cells in response to different aging and rejuvenating paradigms. RNAge can also be used as a drug discovery tool to identify tissue-specific drivers of cellular age. An in silico screen was designed (outlined in FIG. 13A) using the LINCS L1000 dataset. This is a large dataset of transcriptional changes induced by >1 million pharmacological compounds or genetic perturbations in a diverse set of cell lines. Given the evidence for cell-type specificity of the aging signatures presented, the screen was restricted to a smaller subset of perturbations performed in the cell types that are most similar to the primary tissues used to derive our fibroblast and neuronal age signatures (1HAE and NEU). 1HAE is a normal fibroblast line while NEU corresponds to iPSC-derived neurons. The analysis was further restricted to drug-based perturbations as these are more amenable for widespread use as a tool in iPSC-based disease modeling. By restricting the analysis to those parameters, the L1000 dataset included a total of 348 and 3968 perturbations for 1HAE and NEU respectively. The aging scores were calculated for each of those perturbations relative to matched control samples in the dataset, and averaged to get a single value for each perturbation. Aging scores fell on a normal distribution for all cell types and sub-scores (FIGS. 13B and 13C) indicating that most drug treatments did not induce changes in the aging score. However, there were several compounds that rejuvenated or induced transcriptional age (>3 std from mean RNAage score).

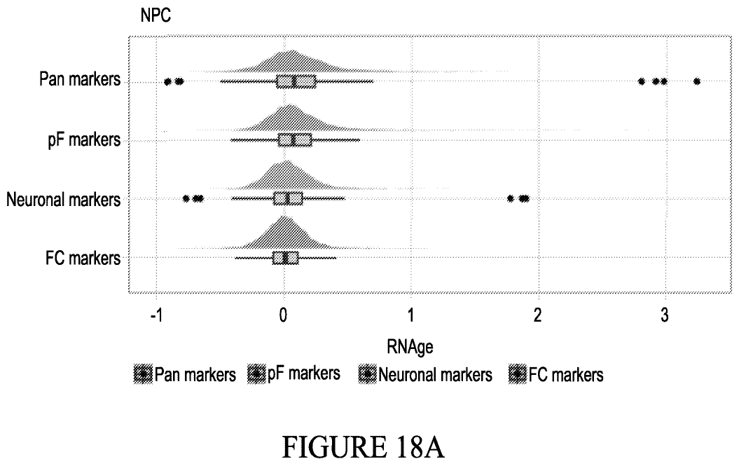

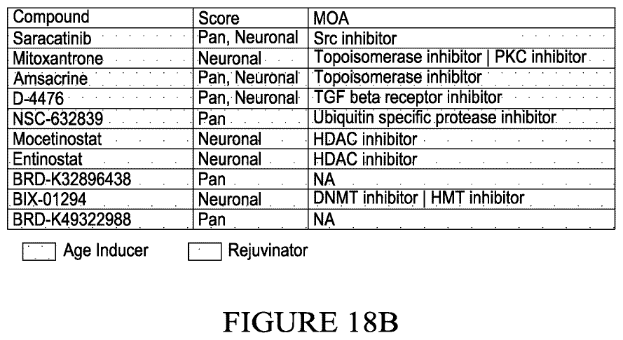

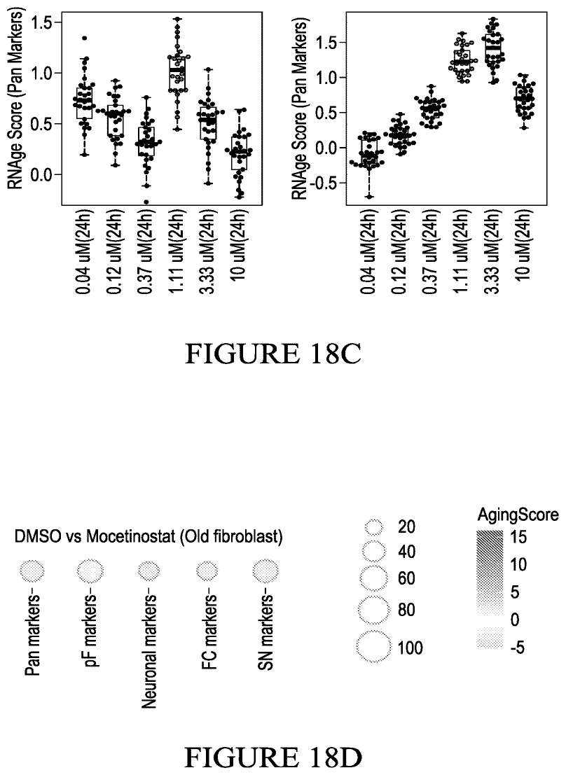

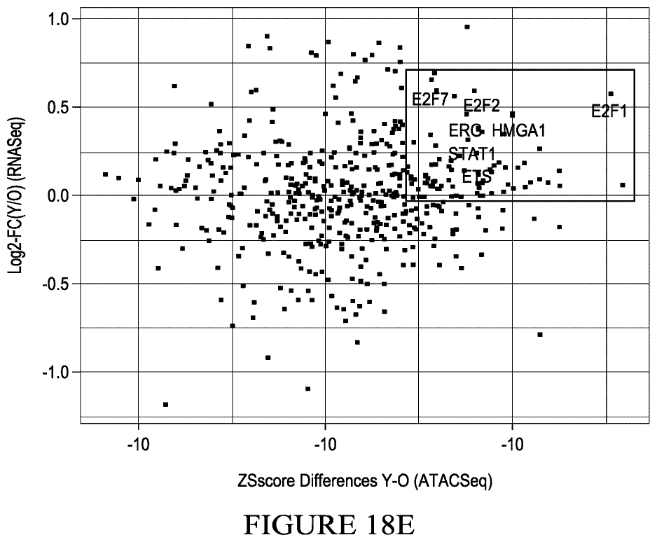

HDAC inhibitors induce rejuvenation. The screening strategy can identify candidate rejuvenating drugs. Interestingly, HDAC inhibitors (HDAC-i) where among the top scoring rejuvenating compounds across both cell types tested (FIGS. 13B-13E). To see how consistent this finding was, the RNAge score was calculated for all compounds added to NPCs (neural progenitor cells), an additional CNS relevant cell type present in the L1000 library. HDAC-i were also the top scoring rejuvenating compounds (Neuronal subscore) in this cell type. To experimentally validate HDAC-i as a regulator of cellular rejuvenation, treated aged primary fibroblasts were treated with Mocetinostat for 24 h to maintain consistency with the L1000 data generation (FIG. 18C). The samples were profiled by RNA-seq data to calculate the RNAge score for Mocetinostat relative to the DMSO control. Validation experiments were performed in fibroblasts due to the availability of primary fibroblasts isolated from aged individuals which is not readily feasible for primary human neurons. Mocetinostat treatment was able to reverse cellular aging in old primary fibroblasts across all RNAge sub scores (FIG. 18D). To further validate this finding, ATAC-seq was performed on primary young and old fibroblasts and results were overlayed with the DEGs identified by RNAseq to identify the top candidate regulators of age in primary fibroblasts (FIG. 18E). This showed motifs for E2F factors, HMGA1 and STAT1 to be differentially accessible in young versus old primary fibroblasts and expressed at a higher level in young vs old fibroblasts. Finally, these transcription factors are also regulated by HDACs. These data indicate that RNAage is suitable to identify candidate rejuvenating factors and provides further impetus to study HDAC-i as potential compounds to promote transcriptional features of cellular rejuvenation.

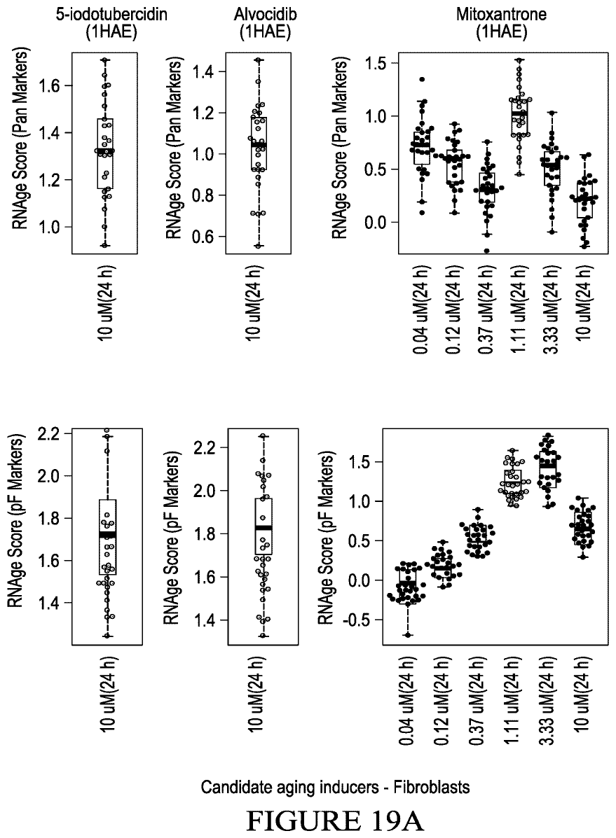

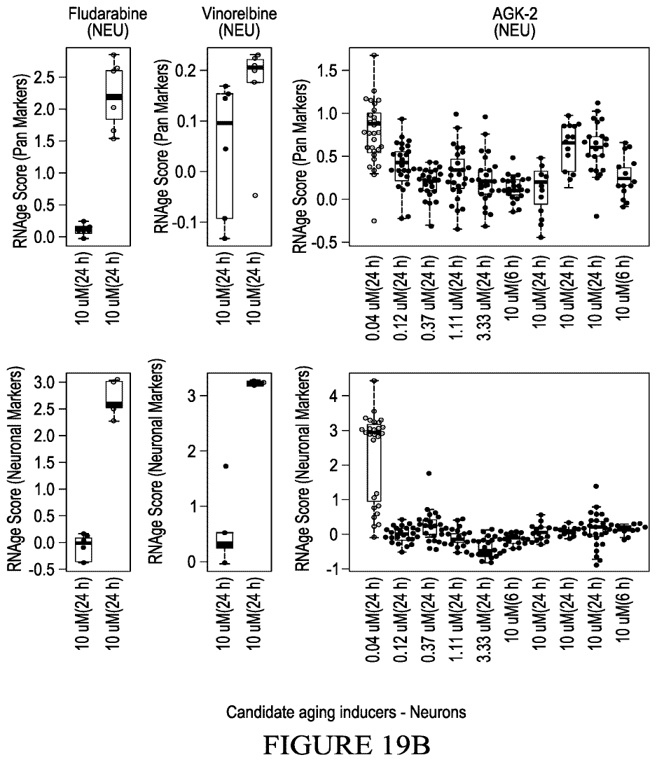

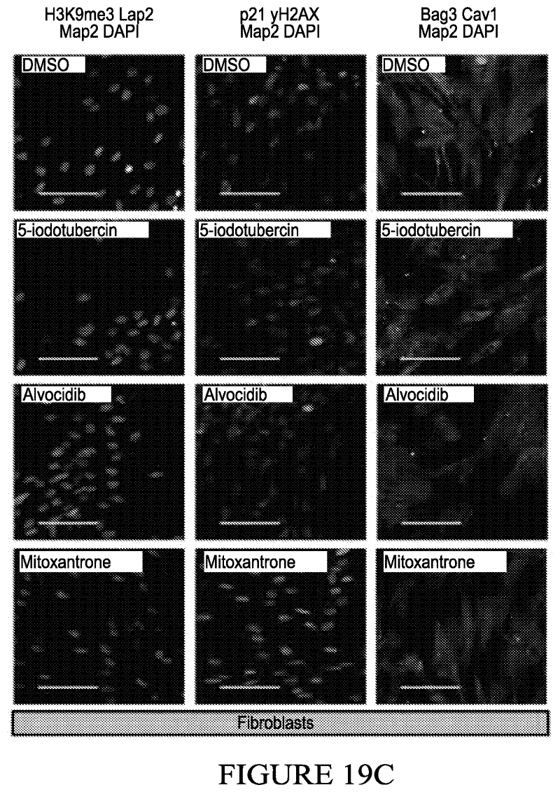

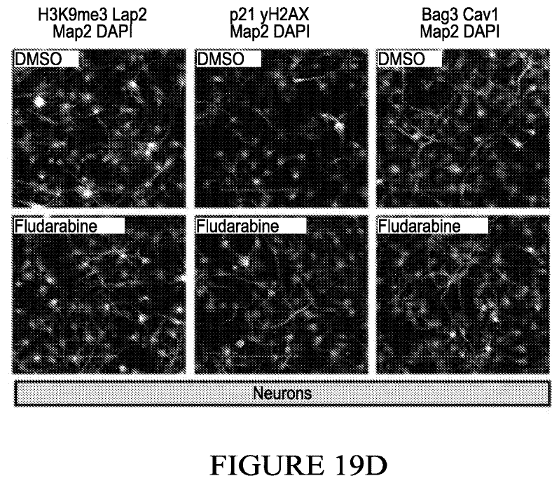

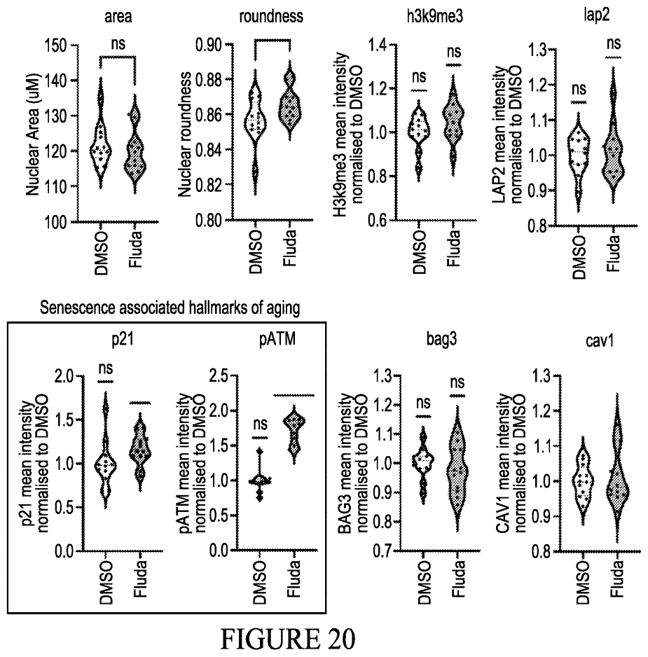

Identification of candidate age inducers using the L1000 dataset. Certain compounds induced an increase in RNAage in fibroblast and in neurons for the L1000 dataset (FIG. 13B-13E). For further experimental validation in fibroblast top scoring compounds were selected including Alvocidib (CDK inhibitor), 5-iodotubercin (adenosine kinase inhibitor) and Mitoxantrone (Topoisomerase II inhibitor), as these three drugs were among the top four age inducers for both the ‘Pan’ and ‘pF’ aging scores. Fludarabine (STAT1 and DNA synthesis inhibitor), Vinorelbine (microtubule antagonist) and AGK-2 (SIRT2 inhibitor) were selected for further validation in neurons, as these were the top three candidate regulators of the pan-neuronal score. Fludarabine was also the top candidate as measured by the ‘Pan’ aging score. To select an appropriate dose for experimental validation, the L1000 dataset was first analyzed, which included data sets across multiple independent perturbations with varying drug concentrations and treatment lengths for many candidate age regulators (FIG. 19A-19B).

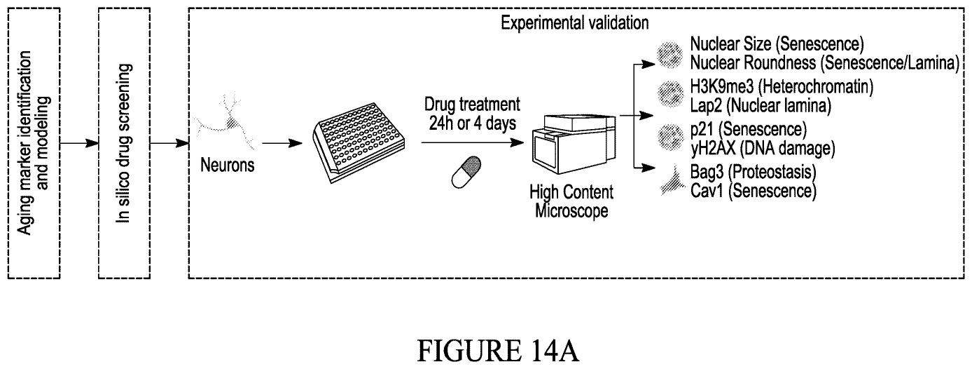

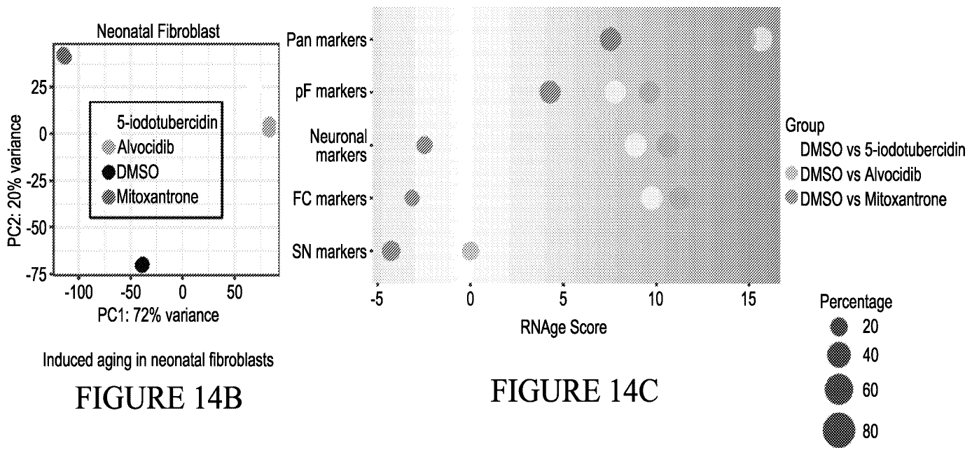

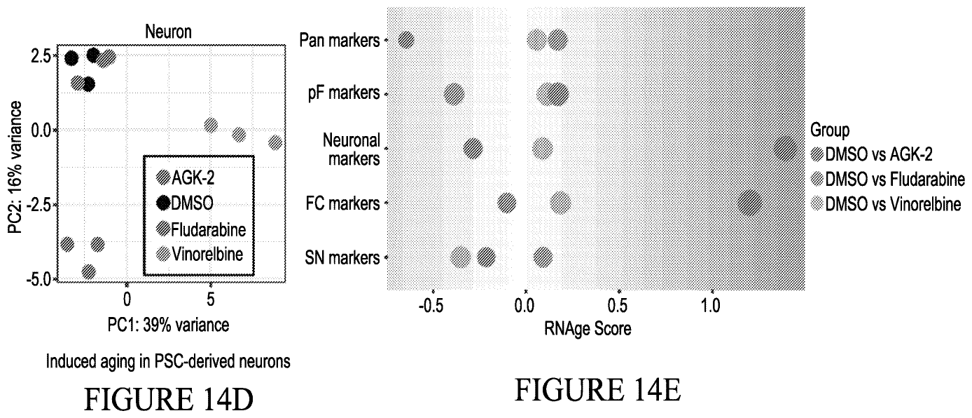

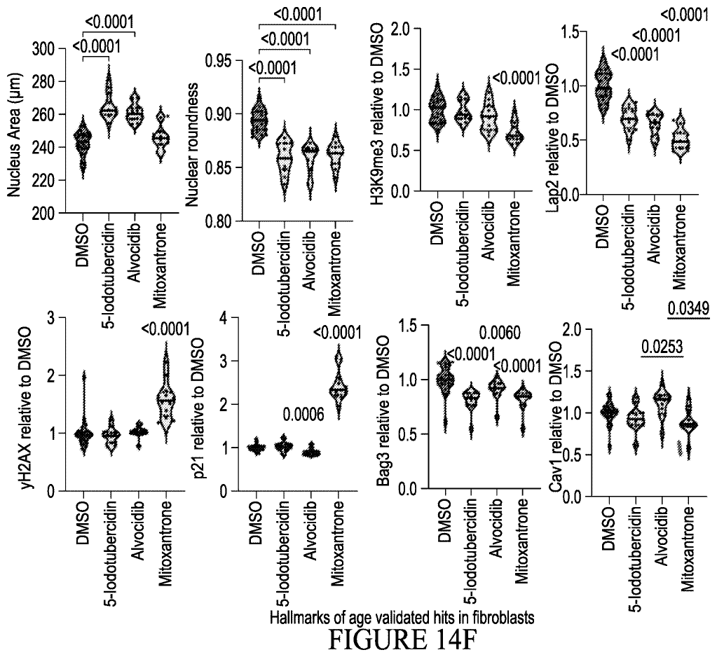

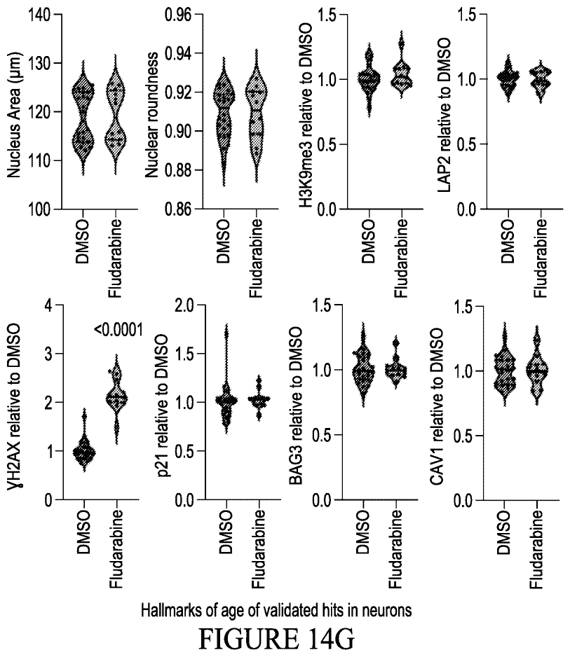

We treated the fetal human dermal fibroblasts (HDF-f) with all three of the candidate age inducers and performed the total RNA sequencing. Principal component analysis (FIG. 14B) of the RNA-seq data indicated that all the fibroblast aging inducers segregated from the control, with Alvocidib and 5-iodotubercin clustering closely together. Next, the RNA-seq data were used to calculate the RNAge score for each of the perturbations relative to DMSO (FIG. 14C). All three of the candidate drugs resulted in an increased age score confirming the in silico scoring results (FIG. 19A). Alvocidib and 5-iodotubercin, which clustered together on the PCA plot resulted in a very similar pattern of induction of the RNAge subscores whereas mitoxantrone was distinct and only showed induced aging in the ‘Pan’ and ‘pF’ aging categories.

The PSC-derived neurons were next treated with the corresponding candidate age inducing compounds and total RNA sequencing was performed. Principal component analysis for the putative inducers of neuronal age showed that Fludarabine and Vinorelbine induced distinct transcriptional changes in neurons (FIG. 14D). AGK2 treatment had no obvious transcriptional effect or impact on the RNAge score (FIG. 14D). Vinorelbine-treated neurons showed only a slight, albeit consistent, induction of aging scores across the aging signatures (FIG. 14D). In contrast, Fludarabine strongly induced aging in neurons based on both the ‘Neuronal’ and ‘FC’ subscores and showed a weak induction of the ‘Pan’ aging score (FIG. 14D). These results indicate that Fludarabine can be an effective tool to induce age-like transcriptional states in young hPSC-derived neurons.

Correlation of RNAge to the hallmarks of cellular age. Next, the age modifying drug treatments were assessed to determine whether they also trigger the well described cellular hallmarks of age (FIG. 14A) including loss of heterochromatin (H3K9me3 and LAP2), disrupted nuclear lamina (decreased nuclear roundness and loss of LAP2), increased DNA damage (γH2AX), increased cellular senescence (p21 and nuclear area), disrupted proteostasis and increased dependence on autophagy (BAG3) and increased CAV1 (multiple functions, broadly increased with age).

Alvocidib, 5-iodotubercin and Mitoxantrone (24h) all increased the relative transcriptional age of fetal fibroblasts (FIG. 14C). All those interventions also decreased BAG3 and induced a loss of nuclear roundness and loss of LAP2 showing that decreased autophagy and nuclear lamina defects are a conserved feature of increased cellular age in fibroblasts (FIG. 14F and FIG. 19C). Treatment with Alvocidib and 5-iodotubercin also induced an increase in nuclear area (FIG. 14F). In contrast, mitoxantrone treatment increased γH2AX and p21 and decreased H3K9me3 immunoreactivity (FIG. 14F). This subset of hallmarks is strongly associated with cellular senescence. These results indicate that within a specific cell type our aging score can be associated with distinct subsets of the aging hallmarks. In neurons, 24 h of Fludarabine treatment was experimentally validated to induce a transcriptional age (FIG. 14E). However, the DNA damage marker yH2AX was the only cellular hallmark of age that changed in response to this treatment at the 24 h time point (FIG. 14G and FIG. 19D).

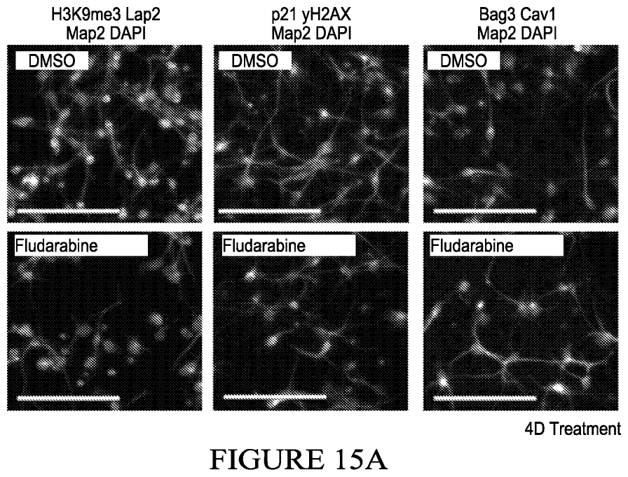

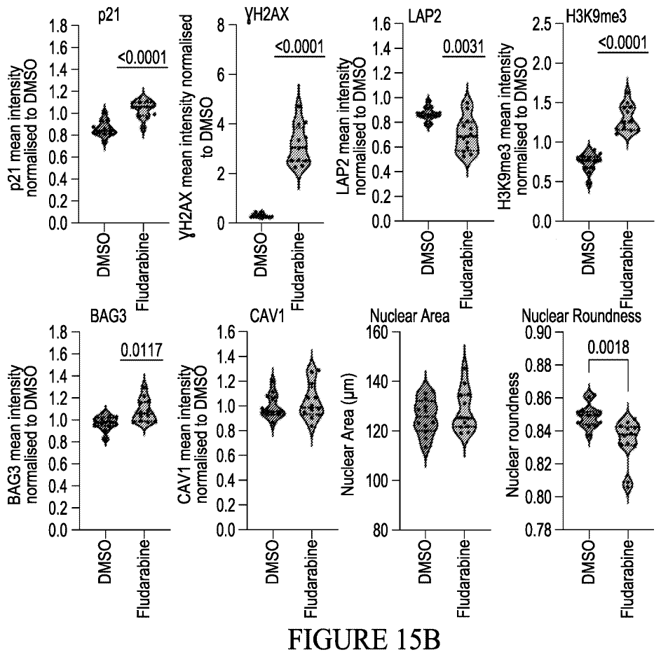

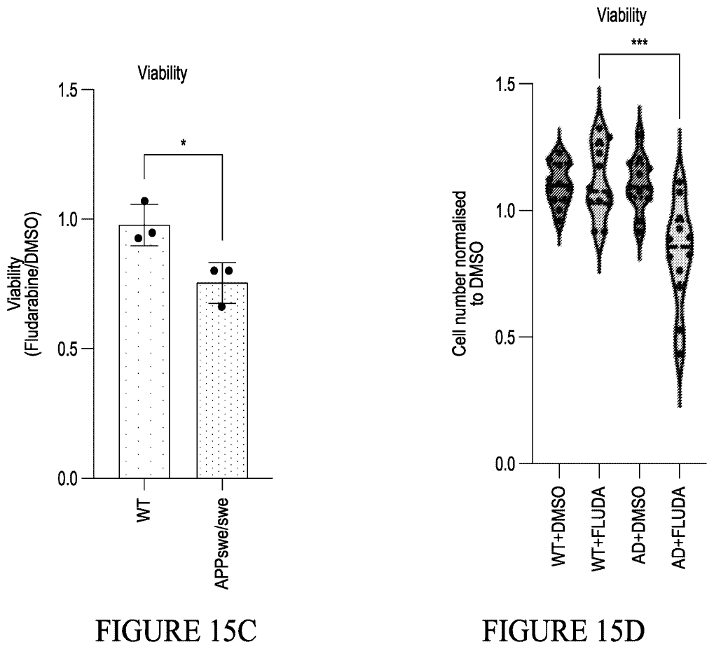

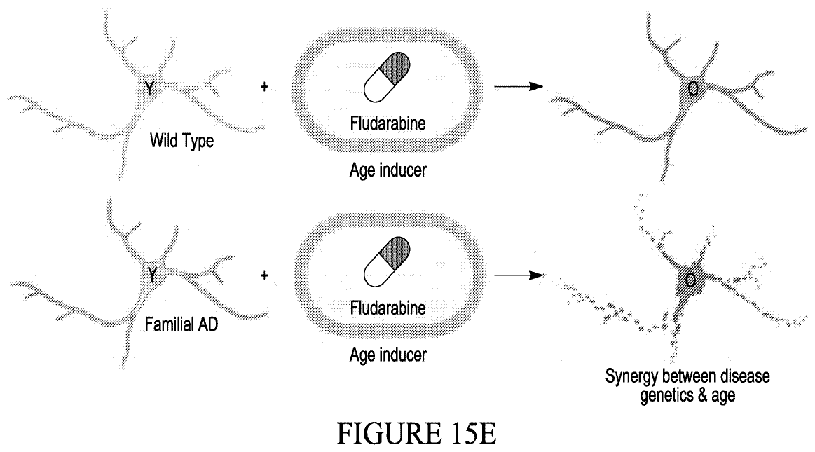

Time dependent induction of cellular hallmarks of aging. It was not clear whether the limited induction of cellular aging hallmarks upon Fludarabine treatment was because the transcriptional aging in neurons is not always associated with the induction of many canonical hallmarks of cellular age or, because transcriptional changes precede the widespread induction of the cellular aging hallmarks. To test this, Fludarabine treatment was extended from 24 h to 4 days and repeated our analysis (FIG. 15A-15B). Under these conditions, yH2AX was further induced and several additional canonical hallmarks of aging were also induced including p21 immunoreactivity, loss of LAP2, increased BAG3 and a decrease in nuclear roundness. However, CAV1 and nuclear area were unchanged whereas H3K9me3 was increased rather than decreased.

Using novel aging strategies to model late-onset disease. A key application of induced aging strategies is their incorporation into PSC-based models of disease to enable the study of late onset phenotypes in a dish. Traditional PSC-derived models of AD are effective at modelling changes in APP processing that are associated with early-onset, genetic forms of AD but modeling the neurodegenerative phase of the disease has been more challenging. To address this, an isogenic pair of hPSC lines edited to carry the APPswe/swe mutation (Saurat et al., CSC) was differentiated into cortical neurons. After 4 days of treatment with Fludarabine, the timepoint where we observed widespread induction of the hallmarks of age (FIG. 15A-15B), an AD-specific loss of neuronal viability was observed. Those findings were quantified using the presto blue viability assay (FIG. 15C) as well as a quantification of cell number by high content imaging (FIG. 15D). These results show that AD-genetic susceptibility can synergize with Fludarabine-mediated induction of cellular age to trigger genotype-specific neuronal loss, a phenotype that that cannot be captured in most hPSC-based in vitro models of AD (FIG. 15E).

### Discussion

A novel transcriptomic measure of relative age is developed, demonstrated in benchmarking published age inducing strategies, and used for identifying novel age modifying strategies. Interestingly, aging scores showed tissue specificity showing that transcriptional aging, and perhaps the aging process more generally can be tissue dependent. This idea is supported by proteomics-based clocks and by a recent study by Buckley et al., who developed and validated cell type specific aging clocks and showed that the different cell types in the mouse SVZ respond differently to known rejuvenation strategies. However, the transcriptomic clock developed by Buckley et al., was not suitable due to technical limitations. They focused on proliferating neural progenitors and their clock does not capture neuronal aging. Furthermore, the study was limited to the mouse and may not be directly applicable to the human CNS given the prominent species-specific differences in SVZ biology. In contrast, Jung et al demonstrated that it is possible to generate a highly predictive RNA-seq-based human aging clock that can be used universally across tissue types. However, this method showed limited success in predicting cellular age in response to known age-related interventions, such as metformin, rapamycin, and hypoxia. This result indicates that those interventions do not, in fact increase relative age. Alternatively, it suggests a lack of sensitivity for a universal aging clock in detecting age-related perturbations given the evidence for cell- and tissue-specificity of aging mechanisms.

Human PSC-derived lineages are functionally and transcriptionally young (fetal-like) limiting their potential in modeling late onset human disease. Age is the strongest risk factor for the several neurogenerative diseases, and there is evidence that cellular age can also impact disease progression. This study identified Fludarabine as a regulator of transcriptional age in neurons. This raised the question of whether induced age could accelerate the onset and progression of neurodegenerative disease. Importantly, age and genetic risk can indeed synergize to trigger neuronal loss in a genotype specific manner. Therefore, the ability to induce cellular aging in vitro can provide a more physiologically relevant experimental system for early drug development and therapeutic intervention.

Robust transcriptional rejuvenation signatures was observed when comparing old primary fibroblasts with their matched iPSC-derived fibroblast counterparts, in agreement with past studies that focused on the rejuvenation of cellular hallmarks of age. Using the L1000 dataset, HDAC inhibitors were identified as a class of small molecules that was able to reset cellular age. HDAC-i are one of the compounds incorporated into chemical based partial reprogramming strategies and has been shown to improve age and late onset disease phenotypes in mouse models. This acts as a proof of principle that RNAge could also be used to identify and optimize cellular rejuvenation strategies and potentially to identify new ways to increase human health span. RNAge score can be particularly useful in tracking the dynamics of cell fate rejuvenation versus fate reprogramming when optimizing partial reprogramming strategies and assessing therapeutic strategies for age associated diseases.

This study demonstrates the use of aging scores to perform an in silico screen for novel age-inducing perturbations. While the current analysis was limited to the L1000 dataset, a similar strategy could be used to probe any other pharmacological perturbation dataset with transcriptomic readouts such as Drug-Seq or PLATE-Seq or genetic perturbation data including possibly single cell-based strategies such as Perturb-Seq, CRISPR-Seq or CROP-Seq. The hits identified using this strategy in conjunction with the L1000 dataset had a high experimental validation rate indicating broad applicability for finding additional age-related regulators in the future. Using RNAge, the performance of novel age inducers can be directly compared using the RNAge score and tested in conjunction with previously published strategies to establish an optimal strategy to induce aging in PSC-derived neurons. One important avenue to explore will be the maintenance of both a neuronal and fibroblast aging signature upon trans differentiation of old fibroblasts directly into neurons (iNs).

Increased transcriptional age is associated with the induction of canonical aging hallmarks in fibroblasts, whereas in neurons, the induction of those hallmarks was limited to increased DNA damage, upon short-term treatment with a wider subset of the hallmarks of aging induced with extended treatment. Thus, DNA damage can act as the primary driver of age in this specific context and indicates that RNAge can act as an early marker of age modification. This indicates that RNAge could allow the aging field to explore which of the hallmarks of age act as drivers of cellular age in different cell types and conditions and lead to a deeper understanding of the mechanistic underpinngs of the aging process. In another example, induction of transcriptional age in fibroblasts can be associated with two distinct patterns of cellular hallmarks of age. Mitoxantrone showed a strong cellular senescence phenotype with increased p21 expression, increased DNA damage and loss of H3K9me3 in contrast to Alvocidib and 5-Iodotubercin. This indicates that the RNAge score can capture aging triggered by distinct cellular mechanisms.

Human PSCs have become an increasingly important tool for the study of human aging and rejuvenation. This study presents a simple strategy to score manipulations aimed at inducing, reversing or retaining cellular age across several experimental paradigms. The identification of several age-inducing and rejuvenating compounds enhances the available toolbox in the field for directing cellular age on demand. This is of broad utility to disease modelling and the stem cell field as it can allow the study of late-onset disease phenotypes in vitro.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

1. A method for inducing cellular aging in a cell or population of cells, comprising contacting the cell or population of cells with one or more age-inducing agent.

2. The method of claim 1, wherein the cell or population of cells comprise fibroblasts; wherein the one or more age-inducing agent is selected from the group consisting of one or more cyclin-dependent kinase (CDK) inhibitor, one or more adenosine kinase inhibitor, one or more topoisomerase II inhibitor, one or more protein kinase C (PKC) inhibitor, one or more DNA binding agent, and any combination thereof.

3. The method of claim 2, wherein:
a. the one or more CDK inhibitor comprises alvocidib;
b. the one or more adenosine kinase inhibitor comprises 5-iodotubercin;
c. the one or more topoisomerase II inhibitor comprises mitoxantrone;
d. the one or more PKC inhibitor comprises bisindolylmaleimide-ix; and/or
e. the one or more DNA binding agent comprises chromomycin-a3.

4. The method of claim 1, wherein the cell or population of cells comprise neurons, motoneurons, cortical neurons, peripheral sensory neurons, or midbrain dopamine neurons; wherein the one or more age-inducing agent is selected from the group consisting of one or more DNA synthesis inhibitor, one or more microtubule antagonist, one or more Sirtuin-2 (SIRT2) inhibitor, BRD-K25731886, doxercalciferol, and any combination thereof.

5. The method of claim 4, wherein:
a) the one or more DNA synthesis inhibitor comprises fludarabine;
b) the one or more microtubule antagonist comprises vinorelbine; and/or
c) the one or more SIRT2 inhibitor comprises AGK2.

6. The method claim 1, wherein the cell or population of cells are derived from primary cells, fetal cells, pluripotent stem cells, embryonic stem cells, induced pluripotent stem cells, or any combination thereof.

7. The method of claim 1, wherein the cell or population of cells are human.

8. The method of claim 1, wherein:
a) contacting the cell or population of cells with one or more age-inducing agent increases the expression level or presence in the cell or population of cells of one or more chronological marker signature of an old cell;
b) contacting the cell or population of cells with one or more age-inducing agent decreases the expression level or presence in the cell or population of cells of one or more chronological marker signature of a young cell; and/or
c) contacting the cell or population of cells with one or more age-inducing agent induces loss of heterochromatin, disrupted nuclear lamina, increased DNA damage, increased cellular senescence, disrupted proteostasis and increased dependence on autophagy, increased caveolin-1 (CAV1) expression, or any combination thereof.

9. A method for rejuvenating cellular age in a cell or population of cells, comprising contacting the cell or population of cells with one or more rejuvenating agent.

10. The method of claim 9, wherein the cell or population of cells comprise fibroblasts; wherein the one or more rejuvenating agent is selected from the group consisting of one or more histone deacetylase (HDAC) inhibitor, one or more heat shock protein (HSP) inhibitor, one or more adrenergic receptor antagonist, one or more phosphoinositide 3-kinase (PI3K) inhibitor, and any combination thereof.

11. The method of claim 10, wherein:
a) the one or more HDAC inhibitor comprises mocetinostat;
b) the one or more HSP inhibitor comprises radicicol, geldanamycin, or a combination thereof;
c) the one or more adrenergic receptor antagonist comprises alfuzosin; and/or
d) the one or more PI3K inhibitor comprises wortmannin.

12. The method of claim 9, wherein the cell or population of cells comprise neurons, motoneurons, cortical neurons, peripheral sensory neurons, or midbrain dopamine neurons; wherein the one or more rejuvenating agent is selected from the group consisting of one or more Sirtuin (SIRT) activator, one or more HDAC inhibitor, one or more poly ADP ribose polymerase (PARP) inhibitor, one or more ataxia telangiectasia and Rad3-related (ATR) signaling inhibitor, and any combination thereof.

13. The method of claim 12, wherein:
a) the one or more SIRT activator comprises resveratrol;
b) the one or more HDAC inhibitor comprises SB-939, BRD-A19037878, or any combination thereof;
c) the PARP inhibitor comprises veliparib; and/or
d) the ATR signaling inhibitor comprises VE-821.

14. The method of claim 9, wherein the cell or population of cells are derived from primary cells, fetal cells, pluripotent stem cells, embryonic stem cells, induced pluripotent stem cells, or any combination thereof.

15. The method of claim 9, wherein the cell or population of cells are human.

16. The method of claim 9, wherein:
a) contacting the cell or population of cells with one or more rejuvenating agent increases the expression level or presence in the cell or population of cells of one or more chronological marker signature of a young cell;
b) contacting the cell or population of cells with one or more rejuvenating agent decreases the expression level or presence in the cell or population of cells of one or more chronological marker signature of an old cell; and/or
c) contacting the cell or population of cells with one or more rejuvenating agent reduces loss of heterochromatin, disrupted nuclear lamina, DNA damage, cellular senescence, disrupted proteostasis and dependence on autophagy, caveolin-1 (CAV1) expression, or any combination thereof

17. A method of modeling neurodegenerative disease, comprising:
contacting a cell or population of cells with one or more age-inducing agent to obtain an age-modulated cell or population of age-modulated cells; and
contacting the age-modulated cell or population of age-modulated cells with one or more candidate compound.

18. The method of claim 17, further comprising detecting an alteration in at least one of the survival, biological activity, structure or morphology of the age-modulated cell or population of age-modulated cells.

19. The method of claim 18, further comprising selecting as the drug a candidate one or more compound that alters at least one of the survival, biological activity, structure or morphology of the age-modulated cell or population of age-modulated cells.

20. The method of claim 17, wherein the neurodegenerative disease is Parkinson's disease (PD) or Alzheimer's Disease (AD).
